SEQUENCE LISTING (3 sequences)

SEQ ID NO: 1 - DNA, 2240 bp, Homo sapiens
acttccgctcggggcggcggcggtggcggaagtgggagcgggcctggagtcttggccataaagcctgaggcggcggcagcggcggagttggcggcttggagagctcgggagagttccctggaaccagaacttggaccttctcgcttctgtcctccgtttagtctcctcctcggcgggagccctcgcgacgcgcccggcccggagcccccagcgcagcggccgcgtttgaaggatgacctctaggaagaaagtgttgctgaaggttatcatcctgggagattctggagtcgggaagacatcactcatgaaccagtatgtgaataagaaattcagcaatcagtacaaagccacaataggagctgactttctgaccaaggaggtgatggtggatgacaggctagtcacaatgcagatatgggacacagcaggacaggaacggttccagtctctcggtgtggccttctacagaggtgcagactgctgcgttctggtatttgatgtgactgcccccaacacattcaaaaccctagatagctggagagatgagtttctcatccaggccagtccccgagatcctgaaaacttcccatttgttgtgttgggaaacaagattgacctcgaaaacagacaagtggccacaaagcgggcacaggcctggtgctacagcaaaaacaacattccctactttgagaccagtgccaaggaggccatcaacgtggagcaggcgttccagacgattgcacggaatgcacttaagcaggaaacggaggtggagctgtacaacgaatttcctgaacctatcaaactggacaagaatgaccgggccaaggcctcggcagaaagctgcagttgctgagggggcagtgagagttgagcacagagtccttcacaaaccaagaacacacgtaggccttcaacacaattcccctctcctcttccaaacaaaacatacattgatctctcacatccagctgccaaaagaaaaccccatcaaacacagttacaccccacatatctctcacacacacacacacacgcacacacacacacacagatctgacgtaatcaaactccagcccttgcccgtgatggctccttggggtctgcctgcccacccacatgagcccgcgagtatggcagcaggacaagccagcggtggaagtcattctgatatggagttggcattggaagcttattctttttgttcactggagagagagagaactgtttacagttaatctgtgtctaattatctgattttttttattggtcttgtggtctttttaccccccctttcccctccctccttgaaggctaccccttgggaaggctggtgccccatgccccattacaggctcacacccagtctgatcaggctgagttttgtatgtatctatctgttaatgcttgttacttttaactaatcagatctttttacagtatccatttattatgtaatgcttcttagaaaagaatcttatagtacatgttaatatatgcaaccaattaaaatgtataaattagtgtaagaaattcttggattatgtgtttaagtcctgtaatgcaggcctgtaaggtggagggttgaaccctgtttggattgcagagtgttactcagaattgggaaatccagctagcggcagtattctgtacagtagacacaagaattatgtacgccttttatcaaagacttaagagccaaaaagcttttcatctctccagggggaaaactgtctagttcccttctgtgtctaaattttccaaaacgttgatttgcataatacagtggtatgtgcaatggataaattgccgttatttcaaaaattaaaattctcattttctttcttttttttcccccctgctccacacttcaaaactcccgttagatcagcattctactacaagagtgaaaggaaaaccctaacagatctgtcctagtgattttacctttgttctagaaggcgctcctttcagggttgtggtattcttaggttagcggagctttttcctcttttccccacccatctccccaatattgcccattattaattaacctctttctttggttggaaccctggcagttctgctcccttcctaggatctgcccctgcattgtagcttgcttaacggagcacttctcctttttccaaaggtctacattctagggtgtgggctgagttcttctgtaaagagatgaacgcaatgccaataaaattgaacaagaacaatgataaaaaaaa

SEQ ID NO: 2 - RNA, 23 nt, Artificial Sequence
  Synthetic polynucleotide
cgaaacuguuaugauaacgugac

SEQ ID NO: 3 - RNA, 27 nt, Artificial Sequence
  Synthetic polynucleotide
uucacguucagauuauggaugguuccc

(19) United States
(12) Patent Application Publication
(10) Pub. No.: US 2022/0275371 A1
(43) Pub. Date: Sep. 1, 2022

(54) USE OF RAB7 GTPASE (RAB7) INHIBITORS IN ENHANCING PERMEABILITY OF THE BLOOD BRAIN BARRIER (BBB)

(71) Applicant: Children's Medical Center Corporation, Boston, MA (US)

(72) Inventors: Marsha A. Moses, Brookline, MA (US); Golnaz Morad, Boston, MA (US)

(73) Assignee: Children's Medical Center Corporation, Boston, MA (US)

(21) Appl. No.: 17/605,799

(22) PCT Filed: Apr. 23, 2020
(86) PCT No.: PCT/US2020/029556

§ 371 Date: Oct. 22, 2021

Related U.S. Application Data
(60) Provisional application No. 62/837,680, filed on Apr. 23, 2019.

(51) Int. Cl. C12N 15/113; A61K 9/127; A61K 45/06; A61P 35/00; C12Q 1/6886

(52) U.S. Cl. CPC C12N 15/1137; A61K 9/127; A61K 45/06; A61P 35/00; C12Q 1/6886; C12N 2310/141; C12N 2310/14; C12Q 2600/178; C12Q 2600/158

(57) ABSTRACT

Provided herein are compositions and methods for delivery an agent (e.g., diagnostic agent or therapeutic agent) to the brain using an extracellular vesicle comprising the agent and a Rab7 inhibitor. Methods of diagnosing or treating a brain disease are also provided.

### RELATED APPLICATION

This application claims the benefit under 35 U.S.C. § 119(e) of U.S. Provisional Application Ser. No. 62/837,680, entitled “USE OF RAB7 GTPASE (RAB7) INHIBITORS IN ENHANCING PERMEABILITY OF THE BLOOD BRAIN BARRIER (BBB)” filed on Apr. 23, 2019, the entire contents of which is incorporated herein by reference.

### GOVERNMENT SUPPORT

This invention was made with government support under Grant No. R01CA185530, awarded by the National Institutes of Health. The government has certain rights in this invention.

### BACKGROUND

The blood brain barrier (BBB) in healthy brain is a diffusion barrier essential for protecting normal brain and the central nervous system (CNS) function by impeding most compounds from transiting from the blood to the brain and CNS. The BBB has been a great hurdle for brain and CNS drug delivery.

### SUMMARY

The present disclosure is based, at least in part, on the surprising finding that breast cancer-derived extracellular vesicles (e.g., exosomes) increase the efficiency of their transport across the BBB through decreasing the brain endothelial expression of Rab7 GTPase (Rab7). Accordingly, some aspects of the present disclosure relate to compositions and methods for enhancing the permeability of the blood brain barrier (BBB) and enhancing delivery of agents across the BBB by co-delivering an agent and a Rab7 inhibitor using extracellular vesicles. Methods of diagnosing and/or treating brain and CNS diseases are also provided.

Some aspects of the present disclosure provide methods of treating a brain disease, the method comprising administering to a subject in need thereof an effective amount of an extracellular vesicle (EV) comprising a therapeutic agent for the brain disease and a Rab7 GTPase (Rab7) inhibitor.

In some embodiments, the EV is isolated from a cell. In some embodiments, the cell is a stem cell, a bone marrow derived cell, an immune cell, a red blood cell, an epithelial cell, or an endothelial cell. In some embodiments, the EV is an engineered EV. In some embodiments, the EV is an exosome, microvesicle, microparticle, ectosome, oncosome, or apoptotic body.

In some embodiments, the EV encapsulates both the therapeutic agent for the brain disease and the Rab7 inhibitor.

In some embodiments, the Rab7 inhibitor inhibits Rab7 expression. In some embodiments, the Rab7 inhibitor comprises an antisense oligonucleotide that targets Rab7 mRNA. In some embodiments, the anti-sense oligonucleotide is a RNAi molecule. In some embodiments, the RNAi molecule is a siRNA or miRNA. In some embodiments, the Rab7 inhibitor inhibits Rab7 activity. In some embodiments, the Rab7 inhibitor is a small molecule inhibitor.

In some embodiments, the brain disease is selected from the group consisting of: brain cancer, neurologic disorder, psychological disorder, cerebrovascular vascular disorder, brain trauma, and brain infection.

In some embodiments, the brain disease is brain cancer. In some embodiments, the brain cancer is primary brain cancer. In some embodiments, the brain cancer is metastatic brain cancer. In some embodiments, the therapeutic agent is an anti-cancer agent. In some embodiments, the anti-cancer agent is a chemotherapeutic agent or an immunotherapeutic agent. In some embodiments, the anti-cancer agent is an RNAi molecule. In some embodiments, the anti-cancer agent is a gene-editing agent. In some embodiments, the anticancer agent is an Cdc42 inhibitor. In some embodiments, the Cdc42 inhibitor is a GTPase inhibitor. In some embodiments, the anticancer agent is a miR-301 inhibitor.

In some embodiments, the brain disease is a neurologic disorder. In some embodiments, the neurologic disorder is a neurodegenerative disease, a neurobehavioral disease, or a developmental disorder. In some embodiments, the neurodegenerative disease is selected from Alzheimer's disease, Parkinson's disease, Huntington's disease, dementia, amyotrophic lateral sclerosis (ALS), prion disease, and motor neuron disease. In some embodiments, the therapeutic agent is selected from: dopaminergic agent, cholinesterase inhibitor, anti-psychotic drug, anti-inflammatory, and brain stimulant.

In some embodiments, the brain disease is a psychological disorder. In some embodiments, the psychological disorder is post-traumatic stress disorder (PTSD), depressive disorder, major depressive disorder, post-partum depression, bipolar disorder, acute stress disorder, generalized anxiety disorder, obsessive-compulsive disorder, panic disorder, schizophrenia, or trichotillomania. In some embodiments, the therapeutic agent is a psychiatric drug. In some embodiments, the psychiatric drug is selected from anti-depressant, anti-psychotic, mood stabilizer, brain stimulant, and anti-anxiety drug.

In some embodiments, the brain disease is brain trauma. In some embodiments, the therapeutic agent is selected from: anti-inflammatory agent, corticosteroid, coagulant drug, and anti-coagulant drug.

In some embodiments, the brain disease is brain infection. In some embodiments, the therapeutic agent is an anti-infective agent. In some embodiments, the anti-infective agent is selected from: antibiotic, anti-viral agent, anti-fungal agent, anti-parasite agent, and anti-prion antibody.

In some embodiments, the EV is administered via injection or infusion. In some embodiments, the EV is administered intravenously, subcutaneously, intraperitoneally, or intracerebrally.

In some embodiments, the Rab7 inhibitor increases the transfer of the EV across the blood brain barrier. In some embodiments, the Rab7 inhibitor enhances the uptake of the therapeutic agent by the brain. In some embodiments, the subject is human.

Other aspects of the present disclosure provide methods of delivering an agent to the brain of a subject, the method comprising administering to a subject in need thereof an extracellular vesicle (EV) comprising the agent and a Rab7 GTPase (Rab7) inhibitor. In some embodiments, the agent is a therapeutic agent or a diagnostic agent.

Other aspects of the present disclosure provide methods of diagnosing a brain disease, the method comprising administering to a subject in need thereof an extracellular vesicle (EV) comprising a diagnostic agent and a Rab7 GTPase (Rab7) inhibitor.

Further provided herein are compositions comprising an extracellular vesicle (EV) comprising an agent and a Rab7 GTPase (Rab7) inhibitor for delivering the agent to the brain of a subject.

In some embodiments, the EV is isolated from a cell. In some embodiments, the cell is a stem cell, a bone marrow derived cell, an immune cell, a red blood cell, an epithelial cell, or an endothelial cell. In some embodiments, the EV is an engineered EV. In some embodiments, the EV is an exosome, microvesicle, microparticle, ectosome, oncosome, or apoptotic body.

In some embodiments, the EV encapsulates both the therapeutic agent for the brain disease and the Rab7 inhibitor.

In some embodiments, the Rab7 inhibitor inhibits Rab7 expression. In some embodiments, the Rab7 inhibitor comprises an antisense oligonucleotide that targets Rab7 mRNA. In some embodiments, the anti-sense oligonucleotide is a RNAi molecule. In some embodiments, the RNAi molecule is a siRNA or miRNA. In some embodiments, the Rab7 inhibitor inhibits Rab7 activity. In some embodiments, the Rab7 inhibitor is a small molecule inhibitor.

In some embodiments, the brain disease is selected from the group consisting of: brain cancer, neurologic disorder, psychological disorder, cerebrovascular vascular disorder, brain trauma, and brain infection.

In some embodiments, the brain disease is brain cancer. In some embodiments, the brain cancer is primary brain cancer. In some embodiments, the brain cancer is metastatic brain cancer. In some embodiments, the therapeutic agent is an anti-cancer agent. In some embodiments, the anti-cancer agent is a chemotherapeutic agent or an immunotherapeutic agent. In some embodiments, the anti-cancer agent is an RNAi molecule. In some embodiments, the anti-cancer agent is a gene-editing agent. In some embodiments, the anticancer agent is an Cdc42 inhibitor. In some embodiments, the Cdc42 inhibitor is a GTPase inhibitor. In some embodiments, the anticancer agent is an miR-301 inhibitor.

In some embodiments, the brain disease is a neurologic disorder. In some embodiments, the neurologic disorder is a neurodegenerative disease, a neurobehavioral disease, or a developmental disorder. In some embodiments, the neurodegenerative disease is selected from Alzheimer's disease, Parkinson's disease, Huntington's disease, dementia, amyotrophic lateral sclerosis (ALS), prion disease, and motor neuron disease. In some embodiments, the therapeutic agent is selected from: dopaminergic agent, cholinesterase inhibitor, anti-psychotic drug, anti-inflammatory, and brain stimulant.

In some embodiments, the brain disease is a psychological disorder. In some embodiments, the psychological disorder is post-traumatic stress disorder (PTSD), depressive disorder, major depressive disorder, post-partum depression, bipolar disorder, acute stress disorder, generalized anxiety disorder, obsessive-compulsive disorder, panic disorder, schizophrenia, or trichotillomania. In some embodiments, the therapeutic agent is a psychiatric drug. In some embodiments, the psychiatric drug is selected from anti-depressants, anti-psychotic, mood stabilizer, brain stimulant, and anti-anxiety drug.

In some embodiments, the brain disease is brain trauma. In some embodiments, the therapeutic agent is selected from: anti-inflammatory agent, corticosteroid, coagulant drug, and anti-coagulant drug.

In some embodiments, the brain disease is brain infection. In some embodiments, the therapeutic agent is an anti-infective agent. In some embodiments, the anti-infective agent is selected from: antibiotic, anti-viral agent, anti-fungal agent, anti-parasite agent, and anti-prion antibody.

In some embodiments, the EV is administered via injection or infusion. In some embodiments, the EV is administered intravenously, subcutaneously, intraperitoneally, or intracerebrally.

In some embodiments, the Rab7 inhibitor increases the transfer of the EV across the blood brain barrier. In some embodiments, the Rab7 inhibitor enhances the uptake of the therapeutic agent by the brain. In some embodiments, the subject is human.

In some embodiments, the composition further comprises a pharmaceutically acceptable carrier. In some embodiments, the subject is human.

Uses of the composition of any one of the compositions described herein for treating or diagnosing a brain disease are also provided.

Other aspects of the present disclosure provide methods of predicting and/or detecting brain metastasis in a subject having breast cancer, the method comprising isolating an extracellular vesicle (EV) from the subject and detecting in the EV miR-301a-3p, wherein the presence of miR-301a-3p indicates the subject is more likely to develop and/or to have brain metastasis, compared to a subject having breast cancer and an EV where the presence of miR-301a-3p is not detected.

Further provided herein are methods of predicting and/or detecting brain metastasis in a subject having breast cancer, the method comprising isolating an extracellular vesicle (EV) from the subject and detecting in the EV one or more biomarkers selected from the group consisting of: TPBG, MRP, ITA2, MOES, ANXAS, UPAR, 5NTD, ANXA2, ANXA1, ACTB, ITB1, ICAM1, BASP1, EF1G, STMN1, and PROF1, wherein the presence of one or more of the biomarkers in the EV indicates the subject is more likely to develop and/or to have brain metastasis, compared to a subject having breast cancer and an EV where the presence of the biomarkers is not detected or a lower level is detected.

Further provided herein are methods of predicting and/or detecting brain metastasis in a subject having breast cancer, the method comprising isolating an extracellular vesicle (EV) from the subject and detecting in the EV one or more biomarkers selected from the group consisting of: TPBG, MRP, ITA2, MOES, ANXAS, UPAR, 5NTD, ANXA2, ANXA1, ACTB, ITB1, ICAM1, BASP1, EF1G, STMN1, PROF1, and miR-301a-3p, wherein the presence of one or more of the biomarkers in the EV indicates the subject is more likely to develop and/or to have brain metastasis, compared to a subject having breast cancer and an EV where the presence of the biomarkers is not detected or a lower level is detected.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

The accompanying drawings are not intended to be drawn to scale. In the drawings, each identical or nearly identical component that is illustrated in various FIGs. is represented by a like numeral. For purposes of clarity, not every component may be labeled in every drawing.

In the drawings:

FIGS. 1A-1I show that brain metastasis-promoting breast cancer EVs breach the BBB. FIG. 1A shows electron microscopy images of EVs isolated from parental and brain-seeking MDA-MB-231 breast cancer cells (P-EV and Br-EV, respectively). FIG. 1B is a schematic depicting the in vivo brain metastasis study design. FIG. 1C shows the average surface area per brain metastasis (mean±SD; n=8 mice per group); Statistical analysis was performed using Mann-Whitney test. FIG. 1D shows representative H&E images of brain metastases. All metastases demonstrated a vessel co-option pattern of growth (arrows). Scale bar, 50 μm. FIG. 1E shows representative fluorescent microscopy images (×200). FIG. 1F is a graph showing the quantification of the in vitro uptake of TdTom-EVs by the components of the BBB (mean±SD; 3 independent experiments). Statistical analysis was performed using two-way ANOVA with Sidak's multiple comparison tests. FIG. 1G is a schematic showing the EV distribution study design. FIG. 1H shows two representative fluorescence images of anti-GFAP immunostaining of brain sections of mice that received retro-orbital injection of TdTom-Br-EVs. Arrows demonstrate Br-EVs taken up by astrocytes (×400, n=3 mice). FIG. 1I shows the average fluorescence intensity in perfused brain tissue homogenates collected 45 minutes following injection of a combination of PBS or Br-EV injection with 10 KDa Alexa647 dextran and 70 KDa FITC dextran (mean±SD; n=3 mice per group). Statistical analysis was performed using Mann-Whitney test. In all panels, ns, not significant; * P≤0.05; ** P≤0.01; *** P≤0.001.

FIGS. 2A-2J show that Br-EVs cross the brain endothelium via transcytosis. FIGS. 2A-2B show a fold change in luminescent signal in the media from abluminal chamber of a Transwell® BBB model under the effect of temperature (FIG. 2A) and endocytosis inhibition (FIG. 2B) (mean±SD; 3 independent experiments). Statistical analyses were performed using unpaired two-tailed Student's t-test (FIG. 2A) and one-way ANOVA (FIG. 2B) with Tukey's multiple comparison test. FIG. 2C shows the effect of Br-EVs and VEGF (positive control) on the permeability coefficient of the endothelial monolayer to 10 KDa and 70 KDa dextran (mean±SD; 3 independent experiments). Statistical analysis was performed using two-way ANOVA with Sidak's multiple comparison tests. FIG. 2D shows the fold change in luminescence intensity of the density gradient fractions of the media from the abluminal chamber. Luminescent signal was normalized to that of the 30% fraction, which does not contain EVs. Fifteen, and 25% fractions correspond to EV density (mean±SD; 3 independent experiments). FIG. 2E shows the time-dependent increase in fluorescent signal in the abluminal channel of an in vitro BBB chip (mean±SD; 3 independent experiments). Statistical analyses were performed using unpaired t-test with Welch's correction. FIG. 2F shows the effect of Br-EVs on the permeability of the BBB model to 10 KDa and 70 KDa dextran (mean±SD; 3 independent experiments). Statistical analysis was performed using two-way ANOVA with Sidak's multiple comparison tests. FIG. 2G shows fluorescent microscopy images of TdTom-Br-EVs taken up by endothelial cells (left) and astrocytes (right) in the BBB-on-a-chip model. The upper panels of FIG. 2H show representative fluorescent images of the zebrafish brain (area selected by square), 1 hour after EV injection. Arrows demonstrate EVs in brain parenchyma. The lower panels of FIG. 2H show time-lapse images of the interaction of Br-EV-containing endocytic vesicles (arrows) with the endothelial abluminal plasma membrane (3 independent experiments). FIG. 2I shows representative fluorescent images of dextran distribution in zebrafish brain vasculature. FIG. 2J shows the intravascular to extravascular ratio of fluorescence intensity in zebrafish brain following injection of dextran (mean±SD; 10 KDa Dextran, 11 fish per group; 70 KDa Dextran, 14 fish per group; 3 independent experiments combined). Statistical analysis was performed using two-way ANOVA with Sidak's multiple comparison tests. In all panels, ns, not significant; * P≤0.05; ** P≤0.01; *** P≤0.001; **** P≤0.0001.

FIGS. 3A-3J show that Br-EV transcytosis involves caveolin-independent endocytosis, recycling endosomes and basolateral SNAREs. FIG. 3A shows flow cytometry quantification of TdTom-Br-EV uptake by brain endothelial cells in the presence of chemical inhibitors of different pathways of endocytosis (mean±SD; 3 independent experiments). Statistical analysis was performed using unpaired two-tailed Student's t-test. FIG. 3B shows representative fluorescence microscopy images of the colocalization of TdTom-Br-EVs with 70 KDa FITC Dextran (marker of macropinocytosis, left panel) and Alexa647 transferrin (marker of clathrin-dependent endocytosis, right panel) from 3 independent experiments. The bottom panels show magnification of the area selected by the square. Arrows indicate colocalization. Scale bar, 25 μm. FIGS. 3C-3D and FIGS. 3F-3G show representative fluorescence microscopy images of the colocalization of TdTom-Br-EVs with rab 11 (FIG. 3C), DQ-Ovalbumin (FIG. 3D), VAMP-3 (FIG. 3F), and VAMP-7 (FIG. 3G). The right panels show magnification of the area selected by the square. Arrows indicate colocalization. Scale bar, 25 μm. FIG. 3E and FIG. 3H show quantification of the percentage of colocalized Br-EV-containing vesicles with rab11, DQ-Ovalbumin (FIG. 3E) and VAMP-3 and VAMP-7 (FIG. 3H) (mean±SD; 3 independent experiments). Statistical analyses were performed using unpaired two-tailed Student's t-test. FIGS. 3I-3J show representative fluorescence microscopy images of the colocalization of TdTom-Br-EVs with Syntaxin 4 (FIG. 3I) and Snap23 (FIG. 3J) from 3 independent experiments. The right panels show magnification of the area selected by the square. Arrows indicate colocalization. Scale bar, 25 μm. In all panels, ns, not significant; * P≤0.05; ** P≤0.01; *** P≤0.001.

FIGS. 4A-4E show that Br-EVs decrease the astrocyte expression of TIMP-2. FIG. 4A is a schematic showing the EV functional study design. FIG. 4B shows the average concentration of TIMP-2 in brain tissue homogenates measured by a mouse TIMP-2 ELISA (mean±SD; n=6 mice per group). Statistical analysis was performed using Mann-Whitney test. FIG. 4C shows the average fold change in concentration of TIMP-2 in conditioned media of brain endothelial cells, pericytes and astrocytes treated with PBS, P-, and Br-EVs (mean±SD; 3 independent experiments). Statistical analysis was performed using two-way ANOVA with Sidak's multiple comparison tests. FIG. 4D shows representative images of mouse brain sections immunostained with anti-GFAP (upper panels) and anti-TIMP-2 (lower panels). The middle panels represent a colormap of areas of protein enrichment (3 independent experiments). Scale bar, 200 μm. FIG. 4E shows the average fluorescence intensity in perfused brain tissue homogenates collected 45 minutes following injection of a combination of 10 KDa Alexa647 dextran and 70 KDa FITC dextran (mean±SD; n=3 mice per group). Statistical analysis was performed using Mann-Whitney test. In all panels, ns, not significant; * P≤0.05; ** P≤0.01.

FIGS. 5A-5I show that Br-EVs downregulate the endothelial Rab7 to facilitate their transport. FIGS. 5A-5C show western blot images and quantification of rab7 and rab11 expression in brain endothelial cells following treatment with EVs in vitro (mean±SD; duplicates in 3 independent experiments). Statistical analyses were performed using one-way ANOVA with Tukey's multiple comparison test. FIGS. 5D-5E show representative fluorescent microscopy images and quantification of the effect of rab7 KD in brain endothelial cells (upper panel) on the transfer of DQ-Ovalbumin to lysosomes for degradation (middle panel) and the expression of LAMP1 lysosomal marker (lower panel) (mean±SD; 3 independent experiments). Scale bar, 25 μm. Statistical analyses were performed using unpaired two-tailed Student's t-test. FIG. 5F shows western blot images of rab7 knockdown in brain endothelial cells. FIG. 5G shows the flow cytometry quantification of TdTom-Br-EV uptake by brain endothelial cells with or without rab7 KD (mean±SD; 3 independent experiments). Statistical analyses were performed using unpaired two-tailed Student's t-test. FIG. 5H shows representative fluorescent microscopy images of TdTom-Br-EV uptake by rab7 KD brain endothelial cells. FIG. 5I shows the quantification of the size of Br-EV-containing endosomal vesicles (mean±SD; 3 independent experiments). Scale bar, 25 μm. Statistical analyses were performed using unpaired two-tailed Student's t-test. In all panels, ns, not significant; * P≤0.05; ** P≤0.01; *** P≤0.001.

FIGS. 6A-6D show the characterization of the cell lines and the isolated EVs. FIG. 6A shows in vivo luminescent imaging of metastases following intracardiac injection of parental and brain-seeking MDA-MB-231 cells. FIG. 6B shows nanoparticle tracking analysis of the size of P-EVs and Br-EVs. FIG. 6C shows representative western blot images of EV markers CD9, CD63, Alix, and the golgi marker, GM130. FIG. 6D shows the percentage of the mice that developed brain metastases following treatment with PBS, P-EVs, and Br-EVs (n=8 mice per group).

FIGS. 7A-7F show the characterization of the in vitro BBB model and the transcytosed EVs. FIG. 7A is a schematic showing static BBB model preparation and transcytosis experiments. FIG. 7B shows representative images of brain endothelial cells immunostained with anti-ZO-1 antibody following treatment with cAMP and Ro 20-1724 (3 independent experiments). FIG. 7C shows the fold change in permeability coefficient of brain endothelial monolayer to 10 KDa (upper graph) and 70 KDa (lower graph) dextran following treatment with cAMP and Ro 20-1724 (mean±SD; 3 independent experiments). Statistical analysis was performed using one-way ANOVA with Tukey's correction for multiple comparisons. FIG. 7D shows the luminescent intensity of in density fractions following density gradient fractionation of luciferase-labeled Br-EVs. FIG. 7E shows electron microscopy images. FIG. 7F shows the quantification of the size of EVs isolated from the low density (15% Optiprep®) and high density (25% Optiprep®) fractions. Statistical analysis was performed using Student's t-test. In all panels, ns, not significant; * P≤0.05; ** P≤0.01; *** P≤0.001.

FIGS. 8A-8B show colocalization of Br-EVs with caveolin and eea1. FIG. 8A shows representative fluorescence microscopy image of brain endothelial cells immunostained with anti-caveolin 1 antibody from 3 independent experiments. Scale bar, 25 μm. FIG. 8B shows a representative fluorescence microscopy image of brain endothelial cells immunostained with anti-eea1 antibody from 3 independent experiments. Right panels show the magnification of the area in the square. Scale bar, 25 μm.

FIGS. 9A-9F show the in vivo and in vitro effects of EVs on the expression of MMPs and TIMPs. FIGS. 9A-9B are graphs showing an average concentration of MMP-2 (FIG. 9A), MMP-9 (FIG. 9B). FIGS. 9C-9D show MMP-14 (FIG. 9C), and TIMP-1 (FIG. 9D) in brain tissue homogenates measured by ELISA (mean±SD; n=6 mice per group). Statistical analysis was performed using Mann-Whitney test. FIG. 9E shows the fold change in the number of migrated astrocytes in a Transwell® migration assay following pre-treatment with PBS, P- or Br-EVs (mean±SD; 3 independent experiments). Statistical analysis was performed using one-way ANOVA with Tukey's test for multiple comparison. FIG. 9F shows the fold change in the concentration of TIMP-2 in astrocyte conditioned media following treatment with conditioned media from PBS-, P-EV, and Br-EV-treated endothelial cells (mean±SD; 3 independent experiments). Statistical analysis was performed using one-way ANOVA. In all panels, ns, not significant; * P≤0.05; ** P≤0.01; *** P≤0.001.

FIG. 10 shows the specific uptake of Br-EVs by astrocytes depends on the CLIC/GEEC pathway. Chemical inhibition of the canonical pathways of endocytosis including clathrin-dependent pathway (chloropromazine), caveolin-dependent pathway (Filipin), and macropinocytosis (EIPA) did not affect Br-EV uptake whereas inhibition of the CLIC/GEEC pathway through inhibiting CDC42 resulted in significant inhibition of the Br-EVs.

FIGS. 11A-11C show how astrocytes internalize breast cancer-derived EVs through the CLIC/GEEC pathway. FIG. 11A shows electron microscopy images of EVs isolated from parental and brain-seeking MDA-MB-231 breast cancer cells (P-EV and Br-EV, respectively). FIG. 11B shows flow cytometry quantification of TdTom-EV uptake by astrocytes treated with chemical inhibitors of endocytosis pathways (mean±SD; 3 independent experiments). Statistical analysis was performed using unpaired two-tailed Student's t-test (** P≤0.01; *** P≤0.001). FIG. 11C shows representative fluorescence microscopy images of the colocalization of TdTom-EVs with GFP-fused GPI in astrocytes from 3 independent experiments. Scale bar, 25 μm.

FIGS. 12A-12C show that Br-EVs are enriched in interacting partners of the CLIC/GEEC cargo. FIG. 12A is a heatmap visualization of quantitative proteomics analyses demonstrating the significantly differentially expressed proteins (P≤0.05) in Br-EVs vs. P-EVs. FIG. 12B shows the functional enrichment analysis of proteins upregulated in P-EVs and Br-EVs (marked with ‘*’). FIG. 12C shows the quantification of surface localization of membrane-associated proteins upregulated in Br-EVs, CD63 serves as positive control (mean±SD; 3 independent experiments). Statistical analysis was performed using unpaired two-tailed Student's t-test (* P≤0.05).

FIGS. 13A-13J show that miR-301a-3p in breast cancer-derived EVs downregulate astrocyte TIMP-2. FIG. 13A shows complementarity between the seeding sequence of miR-301a-3p and the 3′ UTR of TIMP-2. FIG. 13B shows dual luciferase reporter assay to determine the physical interaction between miR-301a-3p and TIMP-2 3′ UTR (normalized to Renilla luciferase activity, mean±SD; 3 independent experiments). Statistical analysis was performed using unpaired two-tailed Student's t-test. FIG. 13C shows TIMP-2 mRNA levels in astrocytes following treatment with miR-301a-3p mimic (normalized to GAPDH, mean±SD; 3 independent experiments). Statistical analysis was performed using unpaired two-tailed Student's t-test. FIG. 13D shows levels of miR-301a-3p in P-EVs and Br-EVs, measured against a standard curve created by miR-301a-3p mimic (mean±SD; 3 independent experiments). Statistical analysis was performed using unpaired two-tailed Student's t-test. FIG. 13E shows the level of pri/pre or mature miR-301a in astrocytes following treatment with EVs (normalized to U6 expression, mean±SD; 3 independent experiments). Statistical analysis was performed using two-way ANOVA with Sidak's multiple comparison tests. FIG. 13F shows the TIMP-2 level in astrocytes following treatment with EVs (normalized to GAPDH, mean±SD; 3 independent experiments). Statistical analysis was performed using two-way ANOVA with Sidak's multiple comparison tests. FIG. 13G shows the level of miR-301a-3p in brain tissue lysates (normalized to U6 levels, mean±SD; n=6 mice per group). Statistical analysis was performed using Mann-Whitney test. FIGS. 13H and 13I show a correlation analysis between miR-301a-3p and TIMP-2 levels in brain tissue lysates in mice treated with P-EVs (FIG. 13H) and Br-EVs (FIG. 13I) (n=6 mice per group). Correlation coefficient was measure using Pearson's correlation analysis. FIG. 13J shows a Kaplan Meier curve demonstrating the association of miR-301a-3p levels with survival in breast cancer patients from the METABRIC dataset. In all panels, ns, not significant; * P≤0.05; ** P≤0.01; *** P≤0.001.

### DETAILED DESCRIPTION OF CERTAIN EMBODIMENTS

The BBB is primarily composed of endothelial cells, pericytes, and astrocyte end feet. The transportation of molecules across the BBB is tightly regulated. The endothelial cells of the BBB form tight junction complexes that strengthen the attachments between adjacent endothelial cells. This barrier is further reinforced through the crosstalk between endothelial cells and abluminal BBB cells such as astrocytes and pericytes^{15, 16}. As a result, factors with a molecular weight of more than 400 Da, including EVs (>106 Da in size) cannot passively cross the BBB through the paracellular junctions¹⁷. Elucidating the ability of breast cancer-derived EVs to breach an intact BBB and the potential mechanism(s) involved in this process is a prerequisite to understanding the initial events that lead to pre-metastatic modulation of the BBB for future brain metastasis.

It was demonstrated herein that breast cancer-derived EVs can breach an intact blood brain barrier through a transcellular transport mechanism and subsequently change the expression profile of astrocytes to prepare a tumor-supporting microenvironment at the BBB. Surprisingly, it was found that the breast-cancer EVs increase the efficiency of their transcellular transport at least by downregulating Rab7 expression in brain endothelial cells. Further, Cdc42 and TIMP-2 were also shown to be involved in the metastatic niche formation. The findings described herein provide useful tools for delivering agents (e.g., therapeutic agents or diagnostic agents) to the brain using extracellular vesicles, and provide strategies for treating various brain diseases.

Accordingly, some aspects of the present disclosure provide compositions comprising an extracellular vesicle (EV) comprising an agent and a Rab7 GTPase (Rab7) inhibitor for delivering the agent to the brain of a subject. The composition can be used to deliver the agent to the brain, wherein the Rab7 inhibitor enhances the permeability of the BBB and enhances the uptake of the agent by the brain.

An “extracellular vesicle” refers to a nano- and micro-scale bilayered or monolayered vesicle derived from a cell. For example, an EV of the present disclosure may be a cell-derived membranous structures that originate from the endosomal system or is shed from the plasma membrane of cells. EVs are present in biological fluids and are involved in multiple physiological and pathological processes. Non-limiting examples of EVs include: exosomes, microvesicles, microparticles, ectosomes, oncosomes, and apoptotic bodies.

An “exosome” is a cell-derived vesicle that is present in many eukaryotic fluids, including blood, urine, and cultured medium of cell cultures. A “microvesicle” is a circular fragment of plasma membrane ranging from 100 nm to 1000 nm shed from almost all cell types. A “microparticle” is a particle between 0.1 and 100 m in size. Commercially available synthetic microparticles are available in a wide variety of materials, including ceramics, glass, polymers, and metals. An “ectosome” is a large vesicle (e.g., ranging from 100-1000 nm in diameter) assembled at and released from the plasma membrane through outward protrusion or budding. An “oncosome” is an EV that plays a role in cancer cell intercellular communication and contributes to the reprogramming of normal cells. An “apoptotic body” is a vesicle containing parts of a dying cell. Apoptotic bodies can be formed during the execution phase of the apoptotic process, when the cell's cytoskeleton breaks up and causes the membrane to bulge outward.

In some embodiments, an EV is isolated from cells (e.g., a cultured cell). The EV (e.g., an exosome) may be isolated from a range of different cell types, e.g., without limitation, stem cells, bone marrow derived cells, immune cells, red blood cells, epithelial cells, or endothelial cells. In some embodiments, the EV is isolated from a bodily fluid of a subject. In some embodiments, the EV is isolated from the subject's serum, plasma, urine, cerebrospinal fluid, or saliva. Method of isolated EVs from cultured cells are known to those skilled in the art, e.g., as described in Li et al., Theranostics. 2017; 7(3): 789-804, incorporated herein by reference. In some embodiments, the EV is a synthetic or engineered EV (e.g., as described in Sasso et al., Microcirculation. 2017 January; 24(1) and Smith et al., Biogerontology. 2015 April; 16(2): 147-185, incorporated herein by reference).

EVs can be used as drug carriers (e.g., as described in Alvarez-Erviti et al., Nature Biotechnology, volume 29, number 4, 341-347, 2011; and Yang et al., Pharm Res. 2015 June; 32(6): 2003-2014, incorporated herein by reference). In some embodiments, the EVs of the present disclosure encapsulate the agent (e.g., a therapeutic agent or a diagnostic agent for a brain disease) and the Rab7 inhibitor.

“Rab7 GTPase (Rab7)” is a small GTPase encoded by the RAB7A gene. The RAB7A gene belongs to the RAB family of genes, which is a member of the RAS oncogene family. The RAB family proteins are GTPases and act like switch which is turned on and off by GTP and GDP molecules. Rab7 is involved in endocytosis, which is a process that brings substances into a cell. The process of endocytosis works by folding the cell membrane around a substance outside of the cell (for example a protein) and then forms a vesicle. The vesicle is then brought into the cell and cleaved from the cell membrane. Rab7 plays an important role in the movement of vesicles into the cell as well as with vesicle trafficking. Rab7 functions as a key regulator in endo-lysosomal trafficking, governs early-to-late endosomal maturation, microtubule minus-end as well as plus-end directed endosomal migration and positions, and endosome-lysosome transport through different protein-protein interaction cascades. Rab7 is also involved in regulation of some specialized endosomal membrane trafficking, such as maturation of melanosomes through modulation of SOX10 and the oncogene MYC.

An “Rab 7 inhibitor” refers to an agent that inhibits the expression and/or activity of Rab7. In some embodiments, the Rab7 inhibitor inhibits the expression of Rab7. For example, in some embodiments, the Rab7 inhibitor may reduce the expression level and of Rab7 by at least 20%, at least 30%, at least 40%, at least 50%, at least 60%, at least 70%, at least 80%, at least 90%, at least 95%, or 100%, compared to in the absence of the Rab7 inhibitor. In some embodiments, the Rab7 inhibitor reduces the expression level and of Rab7 by 20%, 30%, 40%, 50%, 60%, 70%, 80%, 90%, 95%, or 100%, compared to in the absence of the Rab7 inhibitor.

“Inhibition of gene expression” refers to the absence or observable decrease in the level of protein and/or mRNA product from a target gene (e.g., Rab7). In some embodiments, the agent inhibits the expression of Rab7 without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition as compared to in the absence of the agent. As an example, the efficiency of inhibition may be determined by assessing the amount of gene product in the cell: mRNA may be detected with a hybridization probe having a nucleotide sequence outside the region used for the inhibitory nucleic acid, or translated polypeptide may be detected with an antibody raised against the polypeptide sequence of that region.

In some embodiments, the Rab7 inhibitor inhibits the activity (e.g., GTPase activity) of Rab7. In some embodiments, the Rab7 inhibitor may reduce the activity of Rab7 by at least 20%, at least 30%, at least 40%, at least 50%, at least 60%, at least 70%, at least 80%, at least 90%, at least 95%, or 100%, compared to in the absence of the Rab7 inhibitor. In some embodiments, the Rab7 inhibitor reduces the activity level and of Rab7 by 20%, 30%, 40%, 50%, 60%, 70%, 80%, 90%, 95%, or 100%, compared to in the absence of the Rab7 inhibitor. Methods of measuring Rab7 activity are known in the art, e.g., as described in Sun et al., Methods Mol Biol. 2009; 531:57-69; incorporated herein by reference. Kits for measuring Rab7 activity are commercially available, e.g., from NewEast Biosciences (Catalog #82501).

In some embodiments, an Rab7 inhibitor that reduces the expression level of Rab7 also reduces the activity level of RAB7. Rab7 inhibitors that inhibit the expression level and/or activity level Rab7 may be a nucleic acid, a protein, or a small molecule.

In some embodiments, the Rab7 inhibitor is a nucleic acid. A “nucleic acid” is at least two nucleotides covalently linked together, and in some instances, may contain phosphodiester bonds (e.g., a phosphodiester “backbone”). A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine. Nucleic acids of the present disclosure may be produced using standard molecular biology methods (see, e.g., Green and Sambrook, Molecular Cloning, A Laboratory Manual, 2012, Cold Spring Harbor Press).

In some embodiments, the Rab7 inhibitor is an anti-sense nucleic acid. An “anti-sense nucleic acid” a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Antisense nucleic acids include modified or unmodified RNA, DNA, or mixed polymer nucleic acids, and primarily function by specifically binding to matching sequences resulting in modulation of peptide synthesis (Wu-Pong, November 1994, BioPharm, 20-33). Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

The gene sequence of Rab7 is known. For example, human Rab7 gene sequence has the ID number of NC_000003.12 in NCBI reference sequence database. The mRNA sequence of human Rab 7 is provided as SEQ ID NO: 1.

Human Rab7 mRNA
(NM_004637.5, SEQ ID NO: 1)
ACTTCCGCTCGGGGCGGCGGCGGTGGCGGAAGTGGGAGCGGGCCTGGAGT
CTTGGCCATAAAGCCTGAGGCGGCGGCAGCGGCGGAGTTGGCGGCTTGGA
GAGCTCGGGAGAGTTCCCTGGAACCAGAACTTGGACCTTCTCGCTTCTGT
CCTCCGTTTAGTCTCCTCCTCGGCGGGAGCCCTCGCGACGCGCCCGGCCC
GGAGCCCCCAGCGCAGCGGCCGCGTTTGAAGGATGACCTCTAGGAAGAAA
GTGTTGCTGAAGGTTATCATCCTGGGAGATTCTGGAGTCGGGAAGACATC
ACTCATGAACCAGTATGTGAATAAGAAATTCAGCAATCAGTACAAAGCCA
CAATAGGAGCTGACTTTCTGACCAAGGAGGTGATGGTGGATGACAGGCTA
GTCACAATGCAGATATGGGACACAGCAGGACAGGAACGGTTCCAGTCTCT
CGGTGTGGCCTTCTACAGAGGTGCAGACTGCTGCGTTCTGGTATTTGATG
TGACTGCCCCCAACACATTCAAAACCCTAGATAGCTGGAGAGATGAGTTT
CTCATCCAGGCCAGTCCCCGAGATCCTGAAAACTTCCCATTTGTTGTGTT
GGGAAACAAGATTGACCTCGAAAACAGACAAGTGGCCACAAAGCGGGCAC
AGGCCTGGTGCTACAGCAAAAACAACATTCCCTACTTTGAGACCAGTGCC
AAGGAGGCCATCAACGTGGAGCAGGCGTTCCAGACGATTGCACGGAATGC
ACTTAAGCAGGAAACGGAGGTGGAGCTGTACAACGAATTTCCTGAACCTA
TCAAACTGGACAAGAATGACCGGGCCAAGGCCTCGGCAGAAAGCTGCAGT
TGCTGAGGGGGCAGTGAGAGTTGAGCACAGAGTCCTTCACAAACCAAGAA
CACACGTAGGCCTTCAACACAATTCCCCTCTCCTCTTCCAAACAAAACAT
ACATTGATCTCTCACATCCAGCTGCCAAAAGAAAACCCCATCAAACACAG
TTACACCCCACATATCTCTCACACACACACACACACGCACACACACACAC
ACAGATCTGACGTAATCAAACTCCAGCCCTTGCCCGTGATGGCTCCTTGG
GGTCTGCCTGCCCACCCACATGAGCCCGCGAGTATGGCAGCAGGACAAGC
CAGCGGTGGAAGTCATTCTGATATGGAGTTGGCATTGGAAGCTTATTCTT
TTTGTTCACTGGAGAGAGAGAGAACTGTTTACAGTTAATCTGTGTCTAAT
TATCTGATTTTTTTTATTGGTCTTGTGGTCTTTTTACCCCCCCTTTCCCC
TCCCTCCTTGAAGGCTACCCCTTGGGAAGGCTGGTGCCCCATGCCCCATT
ACAGGCTCACACCCAGTCTGATCAGGCTGAGTTTTGTATGTATCTATCTG
TTAATGCTTGTTACTTTTAACTAATCAGATCTTTTTACAGTATCCATTTA
TTATGTAATGCTTCTTAGAAAAGAATCTTATAGTACATGTTAATATATGC
AACCAATTAAAATGTATAAATTAGTGTAAGAAATTCTTGGATTATGTGTT
TAAGTCCTGTAATGCAGGCCTGTAAGGTGGAGGGTTGAACCCTGTTTGGA
TTGCAGAGTGTTACTCAGAATTGGGAAATCCAGCTAGCGGCAGTATTCTG
TACAGTAGACACAAGAATTATGTACGCCTTTTATCAAAGACTTAAGAGCC
AAAAAGCTTTTCATCTCTCCAGGGGGAAAACTGTCTAGTTCCCTTCTGTG
TCTAAATTTTCCAAAACGTTGATTTGCATAATACAGTGGTATGTGCAATG
GATAAATTGCCGTTATTTCAAAAATTAAAATTCTCATTTTCTTTCTTTTT
TTTCCCCCCTGCTCCACACTTCAAAACTCCCGTTAGATCAGCATTCTACT
ACAAGAGTGAAAGGAAAACCCTAACAGATCTGTCCTAGTGATTTTACCTT
TGTTCTAGAAGGCGCTCCTTTCAGGGTTGTGGTATTCTTAGGTTAGCGGA
GCTTTTTCCTCTTTTCCCCACCCATCTCCCCAATATTGCCCATTATTAAT
TAACCTCTTTCTTTGGTTGGAACCCTGGCAGTTCTGCTCCCTTCCTAGGA
TCTGCCCCTGCATTGTAGCTTGCTTAACGGAGCACTTCTCCTTTTTCCAA
AGGTCTACATTCTAGGGTGTGGGCTGAGTTCTTCTGTAAAGAGATGAACG
CAATGCCAATAAAATTGAACAAGAACAATGATAAAAAAAA

Those skilled in the art will be able to design the anti-sense nucleic acids targeting Rab7 based on the Rab7 gene and/or mRNA sequences, and recognize that the exact length of the antisense nucleic acid and its degree of complementarity with its target will depend upon the specific target selected, including the sequence of the target and the particular bases which comprise that sequence. An anti-sense nucleic acid is generally designed to have partial or complete complementarity with one or more target sequences (i.e., complementarity with one or more transcripts of the Rab7 gene). Depending on the particular target sequence, the nature of the inhibitory nucleic acid and the level of expression of anti-sense nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein.

In some embodiments, the Rab7 inhibitor is a RNA interference (RNAi) molecule. “RNA interference (RNAi) is a biological process in which RNA molecules inhibit gene expression or translation, by neutralizing targeted mRNA molecules.

In some embodiments, the Rab7 inhibitor is a microRNA, a small interfering RNA (siRNA), or a short hairpin RNA (shRNA) that inhibits the expression of Rab7. A “microRNA” is a small non-coding RNA molecule (containing about 22 nucleotides) that functions in RNA silencing and post-transcriptional regulation of gene expression. A “siRNA” is a commonly used RNA interference (RNAi) tool for inducing short-term silencing of protein coding genes. siRNA is a synthetic RNA duplex designed to specifically target a particular mRNA for degradation. A “shRNA” an artificial RNA molecule with a tight hairpin turn that can be used to silence target gene expression via RNA interference (RNAi). Expression of shRNA in cells is typically accomplished by delivery of plasmids or through viral or bacterial vectors.

In some embodiment, vector-based RNAi modalities (e.g., siRNA or shRNA expression constructs) are used to reduce expression of Rab7 in a cell. In some embodiments, an isolated plasmid vector (e.g., any isolated plasmid vector known in the art or disclosed herein) that expresses a RNAi molecule such as an shRNA. The isolated plasmid may comprise a specific promoter operably linked to a gene encoding the small interfering nucleic acid. In some embodiments, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based modalities could be employed to inhibit expression Rab7 in a brain endothelial cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006)). siRNAs with amide-linked oligoribonucleosides have been generated that are more resistant to Si nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006). Other molecules that can be used to inhibit expression of Rab7 include ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). The diverse array of suppression strategies that can be employed includes the use of DNA and/or RNA aptamers that can be selected to target Rab7.

In some embodiments, the Rab7 inhibitor is a protein. The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

In some embodiments, the Rab7 inhibitor is a Rab7 antibody or an antibody fragment. Rab7 antibodies are commercially available, e.g., from Abcam (catalog #ab50533), Cellsignal (catalog #9367S), BioLegend (catalog #899901, 850405, 850403, 850401). One skilled in the art is familiar with methods of producing antibodies against a known antigen (i.e., Rab7).

An “antibody” or “immunoglobulin (Ig)” is a large, Y-shaped protein produced mainly by plasma cells that is used by the immune system to neutralize an exogenous substance (e.g., a pathogens such as bacteria and viruses). Antibodies are classified as IgA, IgD, IgE, IgG, and IgM. “Antibodies” and “antibody fragments” include whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody.

The basic 4-chain antibody unit is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, (e.g., Basic and Clinical Immunology, 8th edition, Daniel P. Stites, Abba I. Ten and Tristram G. Parslow (eds.), Appleton & Lange, Norwalk, Conn., 1994, page 71 and Chapter 6, incorporated herein by reference).

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC).

An “antibody fragment” for use in accordance with the present disclosure contains the antigen-binding portion of an antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

In some embodiments, an antibody fragment may be a Fc fragment, a Fv fragment, or a single-change Fv fragment. The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Single-chain Fv also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference).

In some embodiments, the Rab7 inhibitor is a small molecule (e.g., a chemical inhibitor). A “small molecule,” as used herein, refers to an organic compound, whether naturally-occurring or artificially created (e.g., via chemical synthesis) that has a relatively low molecular weight. Typically, an organic compound contains carbon. An organic compound may contain multiple carbon-carbon bonds, stereocenters, and other functional groups (e.g., amines, hydroxyl, carbonyls, or heterocyclic rings). In some embodiments, small molecules are monomeric organic compounds that have a molecular weight of less than about 1500 g/mol. In some embodiments, the molecular weight of the small molecule is less than about 1000 g/mol or less than about 500 g/mol. In some embodiments, the small molecule is a drug, for example, a drug that has already been deemed safe and effective for use in humans or animals by the appropriate governmental agency or regulatory body.

In some embodiments, the small molecule Rab7 inhibitor (e.g., a chemical inhibitor) is selected from the known small molecule Rab7 inhibitors, e.g., as described in Lam et al., J Immunol. 2016 Nov. 15; 197(10):3792-3805; Saxena et al., Journal of Neuroscience 23 Nov. 2005, 25 (47) 10930-10940; Agola et al., ACS Chem Biol. 2012 Jun. 15; 7(6):1095-108, incorporated herein by reference. In some embodiments, the Rab7 inhibitor is a GTPase inhibitor, e.g., as described in Hong et al., PLoS ONE 10(8): e0134317, incorporated herein by reference. Various Rab7 inhibitors are commercially available, e.g., CID 1067700 (Axonmedchem, Catalog #2184). The Rab7 inhibitors exemplified herein are not considered to be limiting. Any small molecules that inhibit the expression or activity of Rab7 can be used in accordance with the present disclosure.

The composition described herein comprises a EV comprising a Rab7 inhibitor and an agent. In some embodiments, the agent is a therapeutic agent or a diagnostic agent.

A “therapeutic agent” refers to an agent that has therapeutic effects to a disease or disorder (e.g., a brain disease or disorder). A therapeutic agent may be, without limitation, proteins, peptides, nucleic acids, polysaccharides and carbohydrates, lipids, glycoproteins, small molecules, synthetic organic and inorganic drugs exerting a biological effect when administered to a subject, a proteolysis targeting chimera molecule (PROTAC) and combinations thereof. In some embodiments, the therapeutic agent is an anti-inflammatory agent, a vaccine antigen, a vaccine adjuvant, an antibody, and enzyme, an anti-cancer drug (e.g., chemotherapeutic agent or immunotherapeutic agent), a clotting factor, a hormone, a steroid, a cytokine, or an antibiotic.

In some embodiments, the therapeutic agent is an antibody or an antibody fragment. For example, the therapeutic agent may be a monoclonal antibody (e.g., chimeric and/or humanized), an antigen binding portion of an antibody (FAB), a Fc fragment, a Fv fragment, a single-change Fv fragment, single-chain variable fragment (scFv), a single domain antibody (e.g., VHH), a diabody, or an affibody.

The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

An antigen binding fragment (Fab) is the region on an antibody that binds antigens. The Fab is composed of one constant and one variable domain from each of the heavy and light chain polypeptides of the antibody. The antigen binding site is formed by the variable domains of the heavy and light chain antibodies.

A single-chain variable fragment (scFv) is a fusion protein of the variable regions of the heavy (VH) and light chains (VL) of immunoglobulins, connected with a short peptide linker comprising 10-25 amino acids. The linker peptide is usually rich in glycine for flexibility, as well as serine or threonine for solubility, and connects the N-terminus of the VH chain with the C-terminus of the VL chain, or vice versa. The scFv retains the specificity of the original immunoglobulin, despite the addition of the linker and removal of the constant regions. In some embodiments, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference).

A diabody is a dimeric antibody fragment designed to form two antigen binding sites. Diabodies are composed of two single-chain variable fragments (scFvs) in the same polypeptide connected by a linker peptide which is too short (˜3-6 amino acids) to allow pairing between the two domains on the same chain, forcing the domains to pair with complementary domains of another chain to form two antigen binding sites. Alternately, the two scFvs can also be connected with longer linkers, such as leucine zippers.

An affibody is an antibody mimetics engineered to bind to a large number of target proteins or peptides with high affinity, imitating monoclonal antibodies. These molecules can be used for molecular recognition in diagnostic and therapeutic applications.

A single chain antibody refers to an antibody that has only a heavy chain or a light chain, but not both (e.g., a heavy chain-only antibody). It is known that Camilids produce heavy chain-only antibodies (e.g., as described in Hamers-Casterman et al., 1992, incorporated herein by reference). The single-domain variable fragments of these heavy chain-only antibodies are termed VHHs or nanobodies. VHHs retain the immunoglobulin fold shared by antibodies, using three hypervariable loops, CDR1, CDR2 and CDR3, to bind to their targets. Many VHHs bind to their targets with affinities similar to conventional full-size antibodies, but possess other properties superior to them. Therefore, VHHs are attractive tools for use in biological research and therapeutics. VHHs are usually between 10 to 15 kDa in size, and can be recombinantly expressed in high yields, both in the cytosol and in the periplasm in E. coli. VHHs can bind to their targets in mammalian cytosol. A VHH fragment (e.g., NANOBODY®) is a recombinant, antigen-specific, single-domain, variable fragment derived from camelid heavy chain antibodies. Although they are small, VHH fragments retain the full antigen-binding capacity of the full antibody. VHHs are small in size, highly soluble and stable, and have greater set of accessible epitopes, compared to traditional antibodies. They are also easy to use as the extracellular target-binding moiety of the chimeric receptor described herein, because no reformatting is required.

A “diagnostic agent” refers to an agent that is used for diagnostic purpose, e.g., by detecting another molecule in a cell or a tissue. In some embodiments, the diagnostic agent is an agent that targets (e.g., binds) a biomarker known to be associated with a disease (e.g., a nucleic acid biomarker, protein biomarker, or a metabolite biomarker) in a subject and produces a detectable signal, which can be used to determine the presence/absence of the biomarker, thus to diagnose a disease. For example, the diagnostic agent may be, without limitation, an antibody or an antisense nucleic acid.

In some embodiments, the diagnostic agent contains a detectable molecule. A detectable molecule refers to a moiety that has at least one element, isotope, or a structural or functional group incorporated that enables detection of a molecule, e.g., a protein or polypeptide, or other entity, to which the diagnostic agent binds. In some embodiments, a detectable molecule falls into any one (or more) of five classes: a) an agent which contains isotopic moieties, which may be radioactive or heavy isotopes, including, but not limited to, 2H, 3H, 13C, 14C, 15N, 18F, 31P, 32P, 35S, 67Ga, 76Br, 99mTc (Tc-99m), 111In, 123I, 125I, 131I, 153Gd, 169Yb, and 186Re; b) an agent which contains an immune moiety, which may be an antibody or antigen, which may be bound to an enzyme (e.g., such as horseradish peroxidase); c) an agent comprising a colored, luminescent, phosphorescent, or fluorescent moiety (e.g., such as the fluorescent label fluoresceinisothiocyanat (FITC); d) an agent which has one or more photo affinity moieties; and e) an agent which is a ligand for one or more known binding partners (e.g., biotin-streptavidin, His-NiTNAFK506-FKBP). In some embodiments, a detectable molecule comprises a radioactive isotope. In some embodiments, a detection agent comprises a fluorescent moiety. In some embodiments, the detectable molecule comprises a dye, e.g., a fluorescent dye, e.g., fluorescein isothiocyanate, Texas red, rhodamine, Cy3, Cy5, Cy5.5, Alexa 647 and derivatives. In some embodiments, the detectable molecule comprises biotin. In some embodiments, the detectable molecule is a fluorescent polypeptide (e.g., GFP or a derivative thereof such as enhanced GFP (EGFP)) or a luciferase (e.g., a firefly, Renilla, or Gaussia luciferase). In some embodiments, a detectable molecule may react with a suitable substrate (e.g., a luciferin) to generate a detectable signal. Non-limiting examples of fluorescent proteins include GFP and derivatives thereof, proteins comprising chromophores that emit light of different colors such as red, yellow, and cyan fluorescent proteins, etc. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols (Methods of biochemical analysis, v. 47, Wiley-Interscience, and Hoboken, N.J., 2006, and/or Chudakov, D M, et al., Physiol Rev. 90(3):1103-63, 2010, incorporated herein by reference, for discussion of GFP and numerous other fluorescent or luminescent proteins. In some embodiments, a detectable molecule comprises a dark quencher, e.g., a substance that absorbs excitation energy from a fluorophore and dissipates the energy as heat.

In some embodiments, the therapeutic agent and or diagnostic agent are for treating or diagnosing a brain disease (e.g., without limitation, brain cancers, neurologic disorders, psychological disorders, cerebrovascular vascular disorders (such as cerebrovascular incident, vascular malformations and anomalies, moyamoya disease, venous angiomas), brain trauma, and brain infection.

In some embodiments, the therapeutic agent is for treating brain cancer (e.g., primary brain cancer and/or metastatic brain cancer). “Primary brain cancer” refers to a cancer that starts in the brain. “Metastatic brain cancer” means cancer that starts from other parts of the body (e.g., breast cancer, prostate cancer, lung cancer, colorectal cancer, skin cancer).

In some embodiments, the therapeutic agent for treating brain cancer is a chemotherapeutic agent. A “chemotherapeutic agent” refers is a chemical agent or drugs that are selectively destructive to malignant cells and tissues. Non-limiting, exemplary chemopharmaceutically compositions that may be used in accordance with the present disclosure include, Neratinib or lapatinib, Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine.

In some embodiments, the therapeutic agent for treating brain cancer is an immunotherapeutic agent. An “immunotherapeutic agent” refers to an agent that modulates (e.g., suppresses or activates) the immune response to treat a disease. Immunetheraepeutic agents are known to those skilled in the art, e.g., those listed on ncbi.nlm.nih.gov/medgen/2637.

In some embodiments, the immunotherapeutic agent is an immune checkpoint inhibitor. An “immune checkpoint” is a protein in the immune system that either enhances an immune response signal (co-stimulatory molecules) or reduces an immune response signal. Many cancers protect themselves from the immune system by exploiting the inhibitory immune checkpoint proteins to inhibit the T cell signal. Exemplary inhibitory checkpoint proteins include, without limitation, Cytotoxic T-Lymphocyte-Associated protein 4 (CTLA-4), Programmed Death 1 receptor (PD-1), T-cell Immunoglobulin domain and Mucin domain 3 (TIM3), Lymphocyte Activation Gene-3 (LAG3), V-set domain-containing T-cell activation inhibitor 1 (VTVN1 or B7-H4), Cluster of Differentiation 276 (CD276 or B7-H3), B and T Lymphocyte Attenuator (BTLA), Galectin-9 (GALS), Checkpoint kinase 1 (Chk1), Adenosine A2A receptor (A2aR), Indoleamine 2,3-dioxygenase (IDO), Killer-cell Immunoglobulin-like Receptor (KIR), Lymphocyte Activation Gene-3 (LAG3), and V-domain Ig suppressor of T cell activation (VISTA).

Some of these immune checkpoint proteins need their cognate binding partners, or ligands, for their immune inhibitory activity. For example, A2AR is the receptor of adenosine A2A and binding of A2A to A2AR activates a negative immune feedback loop. As another example, PD-1 associates with its two ligands, PD-L1 and PD-L2, to down regulate the immune system by preventing the activation of T-cells. PD-1 promotes the programmed cell death of antigen specific T-cells in lymph nodes and simultaneously reduces programmed cell death of suppressor T cells, thus achieving its immune inhibitory function. As yet another example, CTLA4 is present on the surface of T cells, and when bound to its binding partner CD80 or CD86 on the surface of antigen-present cells (APCs), it transmits an inhibitory signal to T cells, thereby reducing the immune response.

An “immune checkpoint inhibitor” is a molecule that prevents or weakens the activity of an immune checkpoint protein, For example, an immune checkpoint inhibitor may inhibit the binding of the immune checkpoint protein to its cognate binding partner, e.g., PD-1, CTLA-4, or A2aR. In some embodiments, the immune checkpoint inhibitor is a small molecule. In some embodiments, the immune checkpoint inhibitors is a nucleic acid aptamer (e.g., a siRNA targeting any one of the immune checkpoint proteins). In some embodiments, the immune checkpoint inhibitor is a recombinant protein. In some embodiments, the immune checkpoint inhibitor is an antibody. In some embodiments, the antibody comprises an anti-CTLA-4, anti-PD-1, anti-PD-L1, anti-TIM3, anti-LAG3, anti-B7-H3, anti-B7-H4, anti-BTLA, anti-GALS, anti-Chk, anti-A2aR, anti-IDO, anti-KIR, anti-LAG3, anti-VISTA antibody, or a combination of any two or more of the foregoing antibodies. In some embodiments, the immune checkpoint inhibitor is a monoclonal antibody. In some embodiments, the immune checkpoint inhibitor comprises anti-PD1, anti-PD-L1, anti-CTLA-4, or a combination of any two or more of the foregoing antibodies. For example, the anti-PD-1 antibody is pembrolizumab (Keytruda®) or nivolumab (Opdivo®) and the anti-CTLA-4 antibody is ipilimumab (Yervoy®). Thus, in some embodiments, the immune checkpoint inhibitor comprises pembrolizumab, nivolumab, ipilimumab, or any combination of two or more of the foregoing antibodies. The examples described herein are not meant to be limiting and that any immune checkpoint inhibitors known in the art and any combinations thereof may be used in accordance with the present disclosure.

In some embodiments, the therapeutic agent for treating brain cancer is an oligonucleotide (e.g., an siRNA, shRNA, or miRNA targeting an oncogene). An “oncogene” is a gene that in certain circumstances can transform a cell into a tumor cell. An oncogene may be a gene encoding a growth factor or mitogen (e.g., c-Sis), a receptor tyrosine kinase (e.g., EGFR, PDGFR, VEGFR, or HER2/neu), a cytoplasmic tyrosine kinase (e.g., Src family kinases, Syk-ZAP-70 family kinases, or BTK family kinases), a cytoplasmic serine/threonine kinase or their regulatory subunits (e.g., Raf kinase or cyclin-dependent kinase), a regulatory GTPase (e.g., Ras), or a transcription factor (e.g., Myc). In some embodiments, the oligonucleotide targets Lipocalin (Lcn2) (e.g., a Lcn2 siRNA). One skilled in the art is familiar with genes that may be targeted for the treatment of cancer.

In some embodiments, the therapeutic agent is a gene editing agent. A “gene editing agent” refers to an agent that is capable of inserting, deleting, or replacing nucleotide(s) in the genome of a living organism. In some embodiments, a genome editing agent is an engineered nuclease that can create site-specific double-strand breaks (DSBs) at desired locations in the genome. The induced double-strand breaks are repaired through nonhomologous end-joining (NHEJ) or homologous recombination (HR), resulting in targeted mutations (‘edits’). As such, the engineered nucleases suitable for genome-editing may be programmed to target any desired sequence in the genome and are also referred to herein as “programmable nucleases.” Suitable programmable nucleases for genome-editing that may be used in accordance with the present disclosure include, without limitation, meganucleases, zinc finger nucleases (ZFNs), transcription activator-like effector-based nucleases (TALEN), and the CRISPR/Cas system. One skilled in the art is familiar with the programmable nucleases and methods of using them for genome-editing. For example, methods of using ZFNs and TALENs for genome-editing are described in Maeder, et al., Mol. Cell 31 (2): 294-301, 2008; Carroll et al., Genetics Society of America, 188 (4): 773-782, 2011; Miller et al., Nature Biotechnology 25 (7): 778-785, 2007; Christian et al., Genetics 186 (2): 757-61, 2008; Li et al., Nucleic Acids Res 39 (1): 359-372, 2010; and Moscou et al., Science 326 (5959): 1501, 2009, incorporated herein by reference.

In some embodiments, the genome-editing agent is a Clustered regularly interspaced short palindromic repeats (CRISPR)/Cas system (e.g., a Cas9 and a guide RNA). A “CRISPR/Cas system” refers to a prokaryotic adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), incorporated herein by reference.

The anti-cancer agent for treating brain cancer used in accordance with the present disclosure can be any anti-cancer drug known to those skilled in the art, e.g., the drugs listed on www.cancer.gov/about-cancer/treatment/drugs.

In some embodiments, the therapeutic agent for treating brain cancer is a Cdc42 inhibitor. In some embodiments, the Cdc42 inhibitor is a GTPase inhibitor. Any known Cdc42 inhibitors can be used in accordance with the present disclosure, e.g., the Cdc42 inhibitor ML141 (CID-2950007) as described in Hong et al., J. Biol. Chem. 288:8531-8543; the Clostridium difficile toxin B as described in Sehr et al., Biochemistry 37, 5296-5304; and secramine as described in Pelish et al., Nat. Chem. Biol. 2, 39-46, incorporated herein by reference. ML141 is commercially available, e.g., from Sigma-Aldrich (Catalog #SML0407).

In some embodiments, the therapeutic agent for treating brain cancer is for maintaining TIMP-2 level in brain endothelial cells, e.g., by inhibiting an miRNA (miR-301) that downregulates TIM2 level. In some embodiments, therapeutic agent for treating brain cancer is a miR-301 inhibitor. Any known miR-301 inhibitors can be used in accordance with the present disclosure, e.g., the miR-301 inhibitors as described in Zhong et al., Scientific Reports, volume 8, Article number: 13291 (2018); Lu et al., J Cancer 2015; 6(12):1260-1275; and Feng et al., J Mol Neurosci (2019) 68: 144, incorporated herein by reference.

In some embodiments, the therapeutic agent is for treating a neurologic disorder. A “neurologic disorder” refers to any disorder of the nervous system (e.g., central nervous system or peripheral nervous system. Structural, biochemical or electrical abnormalities in the brain, spinal cord or other nerves can result in a range of symptoms. Examples of symptoms include paralysis, muscle weakness, poor coordination, loss of sensation, seizures, confusion, pain and altered levels of consciousness. There are many recognized neurological disorders, including, without limitation, neurodegenerative diseases (e.g., without limitation, Alzheimer's disease, Parkinson's disease, Huntington's disease, dementia, amyotrophic lateral sclerosis (ALS), prion disease, and motor neuron diseases), neurobehavioral diseases, and developmental disorders.

One skilled in the art is familiar with therapeutic agents that treat neurologic disorders. For example, the therapeutic agent for treating a neurologic disorder that may be used in accordance with the present disclosure include, without limitation, dopaminergic agents (e.g., dopamine receptor agonists), cholinesterase inhibitors, antipsychotic drugs, anti-inflammatory agents, and brain stimulants. Any of the known agents for treating neurologic disorders can be used in accordance with the present disclosure.

In some embodiments, the therapeutic agent is for treating a psychological disorder. A “psychological disorder” is also referred to as mental disorders or psychiatric disorder. A psychological disorder is a behavioral or mental pattern that causes significant distress or impairment of personal functioning. Such features may be persistent, relapsing and remitting, or occur as a single episode. Many disorders have been described, with signs and symptoms that vary widely between specific disorders. Non-limiting examples of psychological disorders include, post-traumatic stress disorder (PTSD), depressive disorder, major depressive disorders, post-partum depression, bipolar disorder, acute stress disorder, generalized anxiety disorder, obsessive-compulsive disorder, panic disorders, schizophrenia, and trichotillomania.

One skilled in the art is familiar with therapeutic agents (e.g., psychiatric drug) that treat psychological disorders. Non-limiting examples of psychiatric drug include anti-depressants, anti-psychotics, mood stabilizers, brain stimulants, and anti-anxiety drugs. In some embodiments, the therapeutic agent is for treating brain trauma (also termed “traumatic brain injury”). “Brain trauma” refers to a form of acquired brain injury that occurs when a sudden trauma causes damage to the brain. Symptoms of brain trauma can be mild, moderate, or severe, depending on the extent of the damage to the brain. A subject with a mild brain trauma may remain conscious or may experience a loss of consciousness for a few seconds or minutes. Other symptoms of mild brain trauma include headache, confusion, lightheadedness, dizziness, blurred vision or tired eyes, ringing in the ears, bad taste in the mouth, fatigue or lethargy, a change in sleep patterns, behavioral or mood changes, and trouble with memory, concentration, attention, or thinking. A subject with a moderate or severe brain trauma may show these same symptoms, but may also have a headache that gets worse or does not go away, repeated vomiting or nausea, convulsions or seizures, an inability to awaken from sleep, dilation of one or both pupils of the eyes, slurred speech, weakness or numbness in the extremities, loss of coordination, and increased confusion, restlessness, or agitation.

One skilled in the art is familiar with therapeutic agents that treat brain trauma. Non-limiting examples of therapeutic agents that treat brain trauma include anti-inflammatory agents, corticosteroids, and coagulant agents.

Non-limiting examples of dopaminergic agents include apomorphine, bromocriptine, cabergoline, dihydrexidine (LS-186,899), dopamine, fenoldopam, piribedil, lisuride, pergolide, pramipexole, ropinirole, and rotigotine.

Cholinesterase inhibitors (also termed “acetylcholinesterase inhibitors”) are agents that prevent the breakdown of acetylcholine in the body. Cholinesterase inhibitors have been used to treat neurologic disorders (e.g., Alzheimer's disease and dementia). Non-limiting examples of Cholinesterase inhibitors include: organophosphates (e.g., echothiophate, diisopropyl fluorophosphate, cadusafos, chlorpyrifos, cyclosarin, dichlorvos, dimethoate, metrifonate, sarin, soman, tabun, diazinon, malathion, parathion, carbamates), carbamates (e.g., aldicarb, bendiocarb, bufencarb, carbaryl, carbendazim, carbetamide, carbofuran, carbosulfan, chlorbufam, chloropropham, ethiofencarb, formetanate, methiocarb, methomyl, oxamyl, phenmedipham, pinmicarb, pirimicarb, propamocarb, propham, propoxur), onchidal, coumarins, physostigmine, neostigmine, pyridostigmine, ambenonium, demecarium, rivastigmine, phenanthrene derivatives, galantamine, caffeine, rosmarinic acid, alpha-pinene, piperidines, donepezil, tetrahydroaminoacridine (THA), edrophonium, huperzine a, ladostigil, ungeremine, lactucopicrin, acotiamide, hybrid/bitopic ligands, dyflos, echothiophate, and parathion. Cholinesterase inhibitors that are in clinical use include, without limitation: Cognex, Namzaric (Pro), Razadyne ER, Aricept ODT (Pro), Reminyl, Exelon (Pro), Aricept (Pro), and Razadyne (Pro).

Any known anti-psychotic drugs may be used in accordance with the present disclosure. Non-limiting examples of antipsychotic drugs include aripiprazole (Abilify), asenapine (Saphris), cariprazine (Vraylar), clozapine (Clozaril), lurasidone (Latuda), olanzapine (Zyprexa), quetiapine (Seroquel), risperidone (Risperdal), and ziprasidone (Geodon), Fluoxetine, Citalopram, Sertraline, Paroxetine, Escitalopram, Clonazepam, Alprazolam, Lorazepam, Methylphenidate, Amphetamine, Dextroamphetamine, Lisdexamfetamine Dimesylate, typical antipsychotics include: Chlorpromazine, Haloperidol, Perphenazine, Fluphenazine, Aripiprazole, Paliperidone, Lurasidone, Carbamazepine, Lamotrigine, and Oxcarbazepine.

An anti-inflammatory agent is a substance that reduces inflammation (redness, swelling, and pain) in the body. Any known anti-inflammatory agents may be used in accordance with the present disclosure, e.g., the anti-inflammatory agents as described in Maroon et al., Surg Neurol Int. 2010; 1: 80; and Dinarello et al., Cell 140, 935-950, Mar. 19, 2010, incorporated herein by reference.

Any known brain stimulants may be used in accordance with the present disclosure. Brain stimulants may be divided into three categories, short-acting, intermediate-acting, and long-acting. Non-limiting examples of short-acting brain stimulants include: Amphetamine/dextroamphetamine (Adderall), Dextroamphetamine (Dexedrine, ProCentra, Zenzedi), Dexmethylphenidate (Focalin), and Methylphenidate (Ritalin). Non-limiting examples of intermediate-acting brain stimulants include: Amphetamine sulfate (Evekeo) and Methylphenidate (Ritalin SR, Metadate ER, Methylin ER). Non-limiting examples of long-acting brain stimulants include: Amphetamine (Adzenys XR-ODT, Dyanavel XR), Dexmethylphenidate (Focalin XR), Dextroamphetamine (Adderall XR), Lisdexamfetamine (Vyvanse), Methylphenidate (Concerta, Daytrana, Jornay PM, Metadate CD, Quillivant XR, Quillichew ER, Ritalin LA), and mixed salts of a single-entity amphetamine product (Mydayis).

Any known anti-depressants may be used in accordance with the present disclosure. Non-limiting examples of anti-depressants include citalopram (Celexa), escitalopram (Lexapro), fluoxetine (Prozac, Sarafem, Selfemra, Prozac Weekly), fluvoxamine (Luvox), paroxetine (Paxil, Paxil CR, Pexeva), sertraline (Zoloft), vortioxetine (Trintellix, formerly known as Brintellix), vilazodone (Viibryd), duloxetine (Cymbalta), venlafaxine (Effexor), desvenlafaxine (Pristiq, Khedezla), levomilnacipran (Fetzima), amitriptyline (Elavil and Endep are discontinued brands in the US), amoxapine, clomipramine (Anafranil), desipramine (Norpramin), doxepin (Sinequan and Adapin are discontinued brands in the US), imipramine (Tofranil), nortriptyline (Pamelor; Aventyl is a discontinued brand in the US), protriptyline (Vivactil), trimipramine (Surmontil), mirtazapine (Remeron), bupropion (Wellbutrin), trazodone, (Desyrel), trazodone extended release tablets (Oleptro), vortioxetine (Trintellix, formerly known as Brintellix), and vilazodone (Viibryd).

A mood stabilizer is a psychiatric drug used to treat mood disorders characterized by intense and sustained mood shifts (e.g., as seen in patients with typically bipolar disorder type I or type II, borderline personality disorder (BPD) and schizoaffective disorder). Any known mood stabilizers may be used in accordance with the present disclosure. Non-limiting examples of mood stabilizes include: lithium (lithium carbonate or lithium citrate), Divalproex (valproic acid or valproate), Carbamazepine, Oxcarbazepine (Trileptal), and Lamotrigine.

Any known anti-anxiety drugs may be used in accordance with the present disclosure. Non-limiting examples of anti-anxiety drugs include: benzodiazepines, citalopram (Celexa), escitalopram (Lexapro), fluoxetine (Prozac), fluvoxamine (Luvox), paroxetine (Paxil, Pexeva), sertraline (Zoloft), duloxetine (Cymbalta), venlafaxine (Effexor XR), amitriptyline (Elavil), imipramine (Tofranil), nortriptyline (Pamelor), isocarboxazid (Marplan), phenelzine (Nardil), selegiline (Emsam), and tranylcypromine (Parnate). Exemplary benzodiazepines include, without limitation, alprazolam (Xanax), clonazepam (Klonopin), chlordiazepoxide (Librium), diazepam (Valium), and lorazepam (Ativan).

Any known corticosteroids may be used in accordance with the present disclosure. Non-limiting examples of corticosteroids include: bethamethasone (Celestone), prednisone (Prednisone Intensol), prednisolone (Orapred, Prelone), triamcinolone (Aristospan Intra-Articular, Aristospan Intralesional, Kenalog), methylprednisolone (Medrol, Depo-Medrol, Solu-Medrol), dexamethasone (Dexamethasone Intensol, DexPak 10 Day, DexPak 13 Day, DexPak 6 Day), hydrocortisone (Cortef), cortisone, ethamethasoneb (Celestone), Methylprednisolone (Medrol, Depo-Medrol, Solu-Medrol), and Fludrocortisone (Florinef).

Any known coagulant agents may be used in accordance with the present disclosure. Non-limiting examples of coagulant agents include: antihemorrhagic agents, ziolites, desmopressin, coagulation factor concentrates, prothrombin complex concentrate, cryoprecipitate and fresh frozen plasma, recombinant activated human factor VII, tranexamic acid and aminocaproic acid.

In some embodiments, the therapeutic agent is for treating brain infection. “Brain infection” can be caused by viruses, bacteria, fungi, protozoa, or parasites. Another group of brain disorders, called spongiform encephalopathies, are caused by abnormal proteins called prions. Brain infection often also involve other parts of the central nervous system, including the spinal cord. In some instances, infections can cause inflammation of the brain (encephalitis). Viruses are the most common causes of encephalitis. Infections can also cause inflammation of the layers of tissue (meninges) that cover the brain and spinal cord—called meningitis. Often, bacterial meningitis spreads to the brain itself, causing encephalitis. Similarly, viral infections that cause encephalitis often also cause meningitis. Technically, when both the brain and the meninges are infected, the disorder is called meningoencephalitis. However, infection that affects mainly the meninges is usually called meningitis, and infection that affects mainly the brain is usually called encephalitis. Usually in encephalitis and meningitis, infection is not confined to one area. It may occur throughout the brain or within meninges along the entire length of the spinal cord and over the entire brain.

In some embodiments, the therapeutic agent for treating brain infection is selected from known anti-infective agents, e.g., antibiotics for treating bacterial infection, anti-viral agents for treating viral infection, or anti-fungal agents for treating fungal infection, or anti-parasite agents to treat parasitic infection. In some embodiments, the brain infection is prion disease and the therapeutic agent for treat prion disease is an anti-prion antibody.

Any known antimicrobial compounds may be used in accordance with the present disclosure. Non-limiting examples of antimicrobial compounds include, without limitation: antibiotics (e.g., beta lactam, penicillin, cephalosporins, carbapenims and monobactams, beta-lactamase inhibitors, aminoglycosides, macrolides, tetracyclins, spectinomycin), antimalarials, amebicides, antiprotazoal, antifungals (e.g., amphotericin beta or clotrimazole), antiviral (e.g., acyclovir, idoxuridine, ribavirin, trifluridine, vidarbine, ganciclovir). Examples of parasiticides include, without limitation: antihalmintics, Radiopharmaceutics, gastrointestinal drugs.

The present disclosure, in some aspects, further provides the use of the compositions for delivering the agent (e.g., a therapeutic agent or a diagnostic agent) to the brain of a subject. In some embodiments, methods of delivering the agent to the brain of a subject comprises administering any one of the composition described herein to a subject in need thereof.

In some embodiments, the composition for delivery is formulated as a pharmaceutical composition. In some embodiments, the pharmaceutical composition further comprises a pharmaceutically acceptable carrier. “Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” may be a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

In some embodiments, the Rab7 inhibitor enhances (e.g., by at least 20%, at least 30%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more) the transport of the EV comprising the agent (e.g., therapeutic agent or diagnostic agent) and the Rab7 inhibitor across the BBB, compared to in the absence of the Rab7 inhibitor. In some embodiments, the Rab inhibitor enhances (e.g., by at least 20%, at least 30%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more) the uptake of the agent (e.g., therapeutic agent or diagnostic agent) by the brain, compared to in the absence of the Rab7 inhibitor.

In some embodiments, the composition comprising an EV comprising a diagnostic agent for a brain disease and a Rab7 inhibitor described here is used for diagnosing a brean disease (e.g., a brain cancer, a neurologic disorder, a psychological disorder, a cerebrovascular vascular disorder, brain trauma, or brain infection). In some embodiments, the composition comprising an EV comprising a therapeutic agent for a brain disease and a Rab7 inhibitor described here is used for treating a brean disease (e.g., a brain cancer, a neurologic disorder, a psychological disorder, a cerebrovascular vascular disorder, brain trauma, or brain infection).

Accordingly, further provided herein are methods of diagnosing a brain disease (e.g., a brain cancer, a neurologic disorder, a psychological disorder, a cerebrovascular vascular disorder, brain trauma, or brain infection), the method comprising administering to a subject in need thereof an extracellular vesicle (EV) comprising any one of the diagnostic agent described herein and a Rab7 GTPase (Rab7) inhibitor. In some embodiments, the method further comprises detecting a signal.

Also provided herein are methods of treat a brain disease (e.g., a brain cancer, a neurologic disorder, a psychological disorder, a cerebrovascular vascular disorder, brain trauma, or brain infection), the method comprising administering to a subject in need thereof an extracellular vesicle (EV) comprising any one of the diagnostic agent described herein and a Rab7 GTPase (Rab7) inhibitor.

In some embodiments, the brain disease is brain cancer (primary brain cancer or metastatic brain cancer) and the therapeutic agent being co-delivered with the Rab7 inhibitor by the EV is an anti-cancer agent (e.g., any one or combination of the chemotherapeutic agents, immunotherapeutic agents, RNAi molecules, gene-editing agents known in the art and/or described herein). For example, in some embodiments, the therapeutic agent being co-delivered with Rab7 inhibitor by the EV is a Cdc42 inhibitor or a miR-301 inhibitor.

In some embodiments, the brain disease is a neurologic disorder (e.g., neurodegenerative diseases such as Alzheimer's disease, Parkinson's disease, Huntington's disease, dementia, amyotrophic lateral sclerosis (ALS), prion disease, and motor neuron diseases, neurobehavioral diseases, or developmental disorders) and the therapeutic agent being co-delivered with the Rab7 inhibitor by the EV is any one or combination of the dopaminergic agents, cholinesterase inhibitors, antipsychotic drugs, anti-inflammatories, brain stimulants known in the art and/or described herein.

In some embodiments, the brain disease is a psychological disorder (e.g., post-traumatic stress disorder (PTSD), depressive disorder, major depressive disorders, post-partum depression, bipolar disorder, acute stress disorder, generalized anxiety disorder, obsessive-compulsive disorder, panic disorders, schizophrenia, or trichotillomania) and the therapeutic agent being co-delivered with the Rab7 inhibitor by the EV is any one or combination of the psychiatric drugs (e.g., anti-depressants, anti-psychotics, mood stabilizers, stimulants, and anti-anxiety drugs) known in the art and/or described herein.

In some embodiments, the brain disease is brain trauma and the therapeutic agent being co-delivered with the Rab7 inhibitor by the EV any one or combination of the anti-inflammatory agents, corticosteroids, and coagulant drugs known in the art and/or described herein.

In some embodiments, the brain disease is brain infection the therapeutic agent being co-delivered with the Rab7 inhibitor by the EV any one or combination of the anti-infective agents (e.g., antibiotics, anti-viral agents, anti-fungal agents, anti-parasite agents, and anti-prion antibodies) known in the art and/or described herein.

The treat or diagnose a brain disease, the EV comprising a diagnostic or a therapeutic agent and a Rab7 inhibitor may be administered to a subject via injection or infusion. In some embodiments, the EV comprising a diagnostic or a therapeutic agent and a Rab7 inhibitor is administered intravenously, subcutaneously, intraperitoneal, or intracerebral. The Rab7 inhibitor enhances (e.g., by at least 20%) the transport across the BBB of the EV and the uptake of the diagnostic or therapeutic agent by the brain.

Further provided herein are methods of predicting and/or detecting brain metastasis in a subject having breast cancer, the method comprising isolating an extracellular vesicle (EV) from the subject and detecting in the EV miR-301a-3p, wherein the presence of miR-301a-3p indicates the subject is more likely to develop and/or to have brain metastasis, compared to a subject having breast cancer and an EV where the presence of miR-301a-3p is not detected.

Also provided herein are method of predicting and/or detecting brain metastasis in a subject having breast cancer, the method comprising isolating an extracellular vesicle (EV) from the subject and detecting in the EV one or more (e.g., 1, 2, 3, 4, 5, 6, 7, 8, 9, 10, 11, 12, 13, 14, 15, or 16) biomarkers selected from the group consisting of: TPBG, MRP, ITA2, MOES, ANXAS, UPAR, 5NTD, ANXA2, ANXA1, ACTB, ITB1, ICAM1, BASP1, EF1G, STMN1, and PROF1, wherein the presence of one or more of the biomarkers in the EV indicates the subject is more likely to develop and/or to have brain metastasis, compared to a subject having breast cancer and an EV where the presence of the biomarkers is not detected or a lower level (e.g., at least 20%, 30%, 40%, 50%, 60% 70%, 80%, 90%, or 99% lower) is detected.

Further provided herein are methods of predicting and/or detecting brain metastasis in a subject having breast cancer, the method comprising isolating an extracellular vesicle (EV) from the subject and detecting in the EV one or more (e.g., 1, 2, 3, 4, 5, 6, 7, 8, 9, 10, 11, 12, 13, 14, 15, 16, or 17) biomarkers selected from the group consisting of: TPBG, MRP, ITA2, MOES, ANXAS, UPAR, 5NTD, ANXA2, ANXA1, ACTB, ITB1, ICAM1, BASP1, EF1G, STMN1, PROF1, and miR-301a-3p, wherein the presence of one or more of the biomarkers in the EV indicates the subject is more likely to develop and/or to have brain metastasis, compared to a subject having breast cancer and an EV where the presence of the biomarkers is not detected or a lower (e.g., at least 20%, 30%, 40%, 50%, 60% 70%, 80%, 90%, or 99% lower) level is detected.

“A therapeutically effective amount” as used herein refers to the amount of each therapeutic agent (e.g., therapeutic agents for treating any of the brain disease described herein) of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons.

Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system. Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art.

In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the anti-cancer agent used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the anti-cancer agent (such as the half-life of the anti-cancer agent, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the anti-cancer agent is administered for preventive or therapeutic purposes, previous therapy, the subject's clinical history and response to the antagonist, and the discretion of the attending physician. Typically the clinician will administer an anti-cancer agent until a dosage is reached that achieves the desired result. Administration of one or more anti-cancer agents can be continuous or intermittent, depending, for example, upon the recipient's physiological condition, whether the purpose of the administration is therapeutic or prophylactic, and other factors known to skilled practitioners. The administration of an anti-cancer agent may be essentially continuous over a preselected period of time or may be in a series of spaced dose, e.g., either before, during, or after developing a disease.

As used herein, the term “treating” refers to the application or administration of an anti-cancer agent to a subject in need thereof. “A subject in need thereof”, refers to an individual who has a brain disease, a symptom of the brain disease, or a predisposition toward the brain disease, with the purpose to cure, heal, alleviate, relieve, alter, remedy, ameliorate, improve, or affect the disease, the symptom of the disease, or the predisposition toward the brain disease.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., rodent (e.g., mouse or rat), primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). The non-human animal may be a male or female at any stage of development. The non-human animal may be a transgenic animal or genetically engineered animal.

In some embodiments, the subject is a companion animal (a pet). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., rats, mice, guinea pigs, and hamsters), rabbits, or non-human primates.

In some embodiments, a “subject in need thereof” refers to a subject that needs treatment of a brain disease (e.g., a brain cancer, a neurologic disorder, a psychological disorder, a cerebrovascular vascular disorder, brain trauma, or brain infection).

Alleviating a disease includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

Conventional methods, known to those of ordinary skill in the art of medicine, can be used to administer the anti-cancer agent the subject, depending upon the type of disease to be treated or the site of the disease. The anti-cancer agent can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1. Breast Cancer-Derived Extracellular Vesicles Breach the Blood Brain Barrier Via Transcytosis to Promote a Pre-Metastatic Niche

Breast cancer is one of the leading causes of metastatic brain tumors¹. The prognosis of breast cancer patients with brain metastasis is extremely poor, with a reported median survival of 10 months². An urgent need exists, therefore, to develop early diagnostics and effective therapeutics for breast to brain metastasis informed by an understanding of the early mechanisms involved in brain metastasis formation.

During the early stages, brain metastases follow a vessel co-option pattern of growth and remain confined to the brain vasculature, the blood brain barrier (BBB)³. As such, the microenvironment surrounding the BBB serves as an initial niche for metastatic tumor cells⁴. Identifying the dynamic changes that occur in the microenvironment around the BBB prior to brain metastasis is essential to understanding the early mechanisms of brain metastasis.

Tumor-derived extracellular vehicles (EVs) have been identified as early contributors to metastasis formation. Once released into the circulation, they transfer a variety of proteins and genetic material to stromal cells in distant organs⁵. These events lead to the modulation of the microenvironment in pre-metastatic organs in support of future metastatic growth⁶⁻⁸. Recent studies suggest that breast cancer-derived EVs can contribute to the pre-metastatic modulation of brain through affecting the components of the BBB⁹⁻¹³. Some of these EV-derived effects were observed in the abluminal components of the BBB such as astrocytes⁹⁻¹¹. These findings raise an important question as to whether breast cancer-derived EVs can breach the BBB, a required step for pre-metastatic niche preparation.

The BBB is primarily composed of brain endothelial cells, pericytes, and astrocyte end feet. The transportation of molecules across the BBB is tightly regulated. Brain endothelial cells form tight junction complexes that strengthen the attachments between adjacent endothelial cells¹⁴. This barrier is further reinforced through the crosstalk between endothelial cells and abluminal BBB cells such as astrocytes and pericytes^{15, 16}. As a result, factors with a molecular weight of more than 400 Da, including EVs (>106 Da in size) cannot passively cross the BBB through the paracellular junctions¹⁷. Elucidating the ability of breast cancer-derived EVs to breach an intact BBB and the potential mechanism(s) involved in this process is a prerequisite to understanding the initial events that lead to pre-metastatic modulation of the BBB for future brain metastasis.

In the present disclosure, it is demonstrated that breast cancer-derived EVs can breach an intact blood brain barrier and the mechanism driving this process is identified. To overcome the challenges associated with studying the complex structure of the BBB, this process was investigated by using a combination of state-of-the-art in vitro and in vivo models of BBB. The present disclosure demonstrates that these EVs cross the BBB through a transcellular transport mechanism and can subsequently change the expression profile of astrocytes to prepare a tumor-supporting microenvironment at the BBB. Provided herein is data suggesting that at least one mechanism by which this process occurs is through alterations in the expression of extracellular matrix (ECM)-remodeling proteins by astrocytes. Moreover, the present disclosure identifies and characterizes mechanisms by which tumor-derived EVs modulate the endocytic pathway in brain endothelial cells to increase the efficiency of their transcellular transport. These findings are the first to elucidate the mechanistic events involved in the transport of breast cancer-derived EVs across the blood brain barrier and in doing so, identify potential targets for early diagnostics and therapeutics for brain metastasis.

### Results

### Brain Metastasis Promoting Breast Cancer EVs Breach the BBB In Vivo

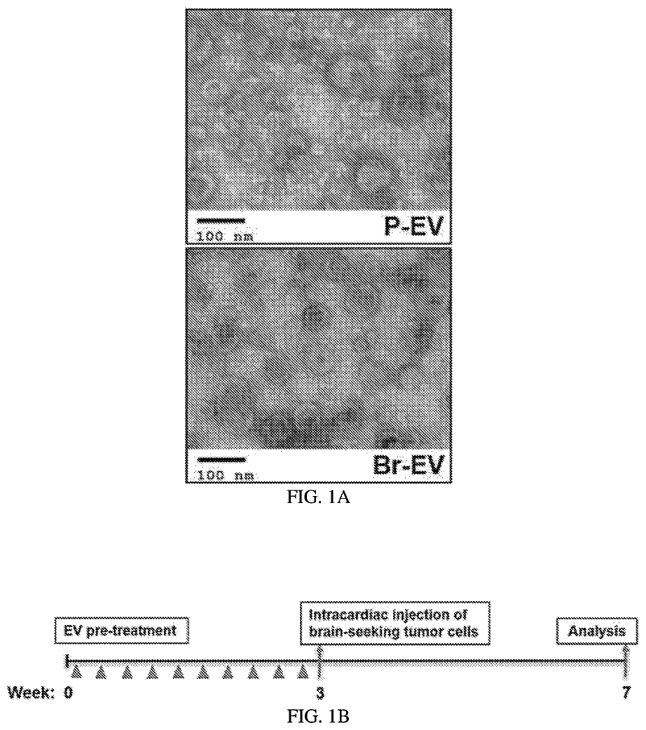

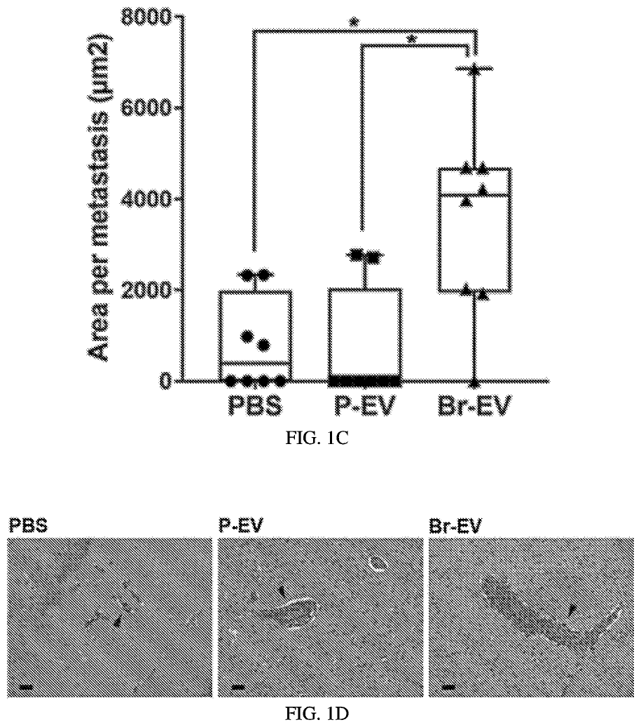

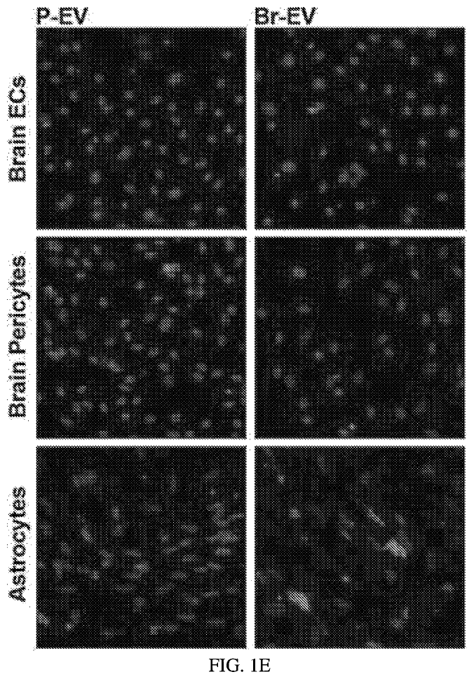

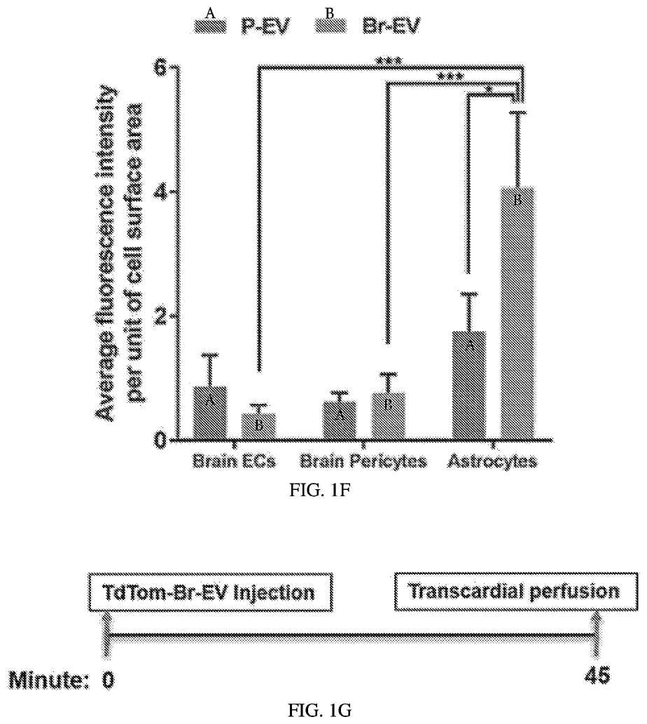

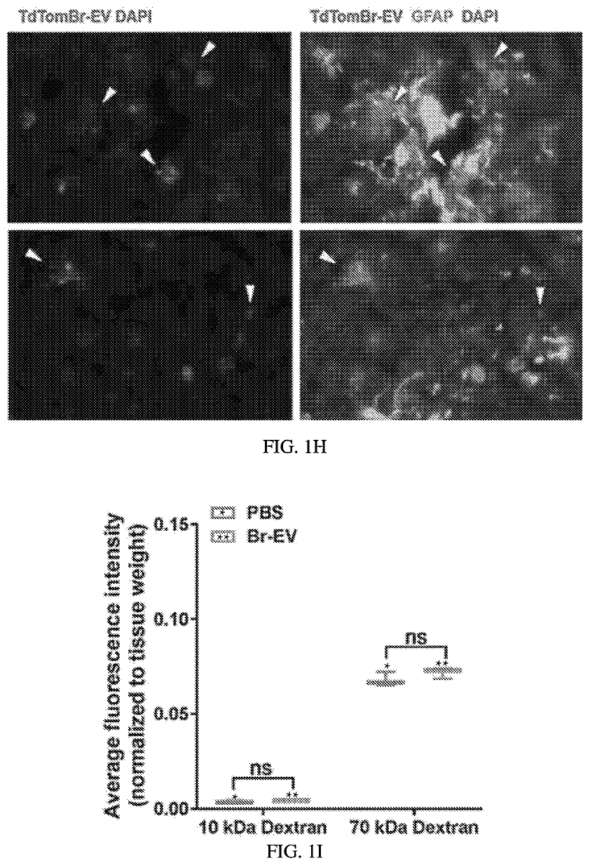

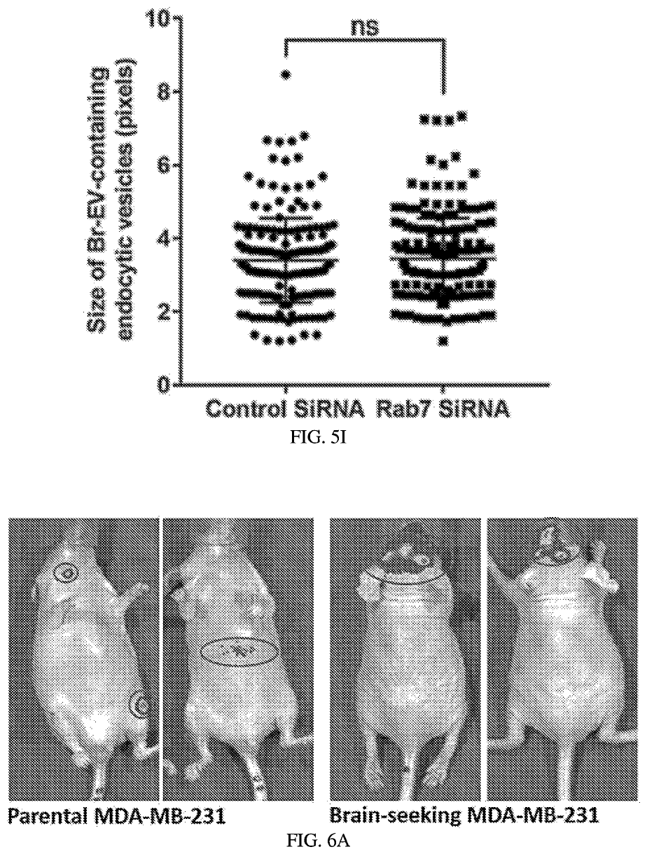

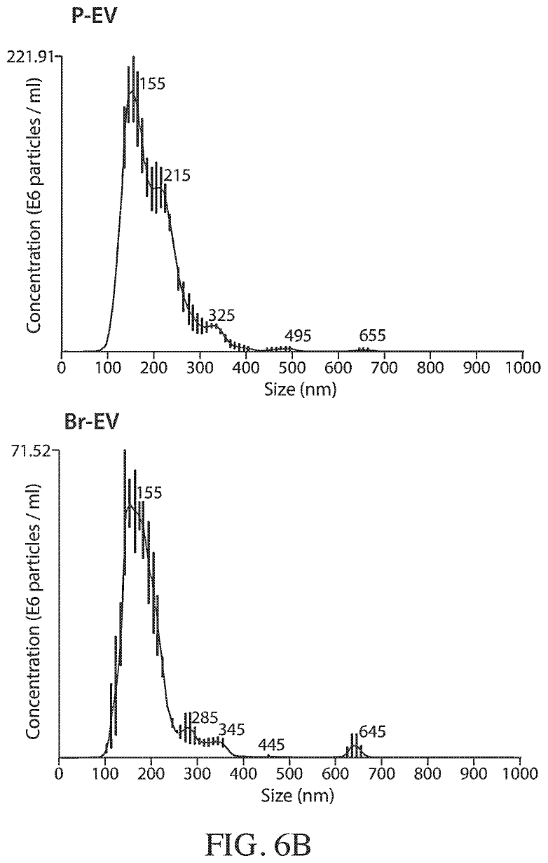

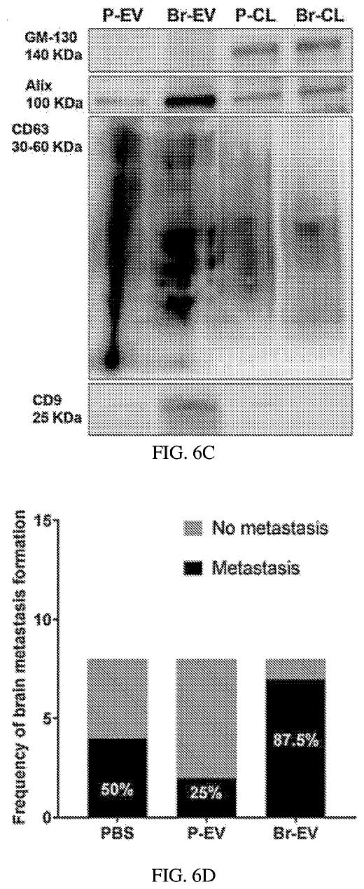

Given the high incidence of brain metastasis in triple negative breast cancer², the parental as well as a brain-seeking variant of the triple negative MDA-MB-231 breast cancer cell line was chosen to study the role of breast cancer-derived EVs in brain metastasis. The pattern of metastasis of these cells was confirmed to be consistent with previous reports¹⁸ (FIG. 6A). A population of EVs defined as small EVs (size <200 nm) or exosomes, was isolated from the parental and brain-seeking cells (P-EVs and Br-EVs, respectively), using the sequential centrifugation technique¹⁹. EVs were characterized according to the guidelines of the International Society for Extracellular Vesicles²⁰. P-EVs and Br-EVs exhibited a lipid bilayer structure (FIG. 1A) and a mode size of 154.1+/−7.0 nm and 158.5+/−6.0 nm, respectively (FIG. 6B). Enrichment of EVs in endosomal markers such as CD63, CD9, and Alix and the lack of detectable GM130, a golgi marker, in EV samples indicated an endosomal origin of these small EVs (FIG. 6C). Using OPTIPREP™ density gradient ultracentrifugation, the density of EVs was found to be within a range of 1.105-1.184 g/ml, consistent with previous reports¹⁹.

Next, the ability of breast cancer-derived EVs to facilitate brain metastasis was tested. Nude mice were pretreated with 3 μg of P- or Br-EVs (˜3-4×10⁹ particles; EVs from approximately 5×10⁵ cells) every two days for a total of 10 retro-orbital injections. This dosage has been shown to be within the concentration range observed for circulating EVs in tumor-bearing mice⁷. After the final EV injection, each mouse received an intracardiac injection of the brain-seeking MDA-MB-231 breast cancer cells (2×10⁵ cells per injection), as described previously¹⁸ (FIG. 1B). At week 4, histological analyses demonstrated that pretreatment with Br-EVs but not P-EVs significantly increased the size of metastases (FIG. 1C). The incidence of brain metastasis also increased in the Br-EV-treated group (FIG. 6D). These findings indicate that Br-EVs can facilitate brain metastasis formation and growth. Consistent with previous reports, a vessel co-option pattern of growth was observed for all brain metastases (FIG. 1D), supporting the role of BBB as an initial niche for tumor cell growth⁴.

To study the interaction of breast cancer-derived EVs with the BBB, the uptake of P-, and Br-EVs by the major components of the BBB was evaluated. TdTomato-labeled EVs (TdTom-P-EVs and TdTom-Br-EVs) were incubated with primary human brain microvascular endothelial cells, brain pericytes, and astrocytes. Astrocytes demonstrated a preferential uptake of the Br-EVs compared to P-EVs (FIG. 1E-1F). The ability of astrocytes to effectively take up Br-EVs suggested a prominent role for these cells in the Br-EV-driven facilitation of brain metastasis. Given the restrictive characteristics of the BBB¹⁴, the ability of Br-EVs to breach the BBB in a mouse model was examined. Retro-orbital injections of the TdTom-Br-EVs (3 μg per mouse) were performed and evaluated the distribution of EVs to the brain (FIG. 1G). Histological analyses demonstrated that Br-EVs were taken up by GFAP+ astrocytes (FIG. 1H), confirming their ability to cross the BBB in vivo. The integrity of the BBB remained unaffected throughout the course of this experiment (FIG. 1I).

### Br-EVs Cross the Brain Endothelium Via Transcytosis

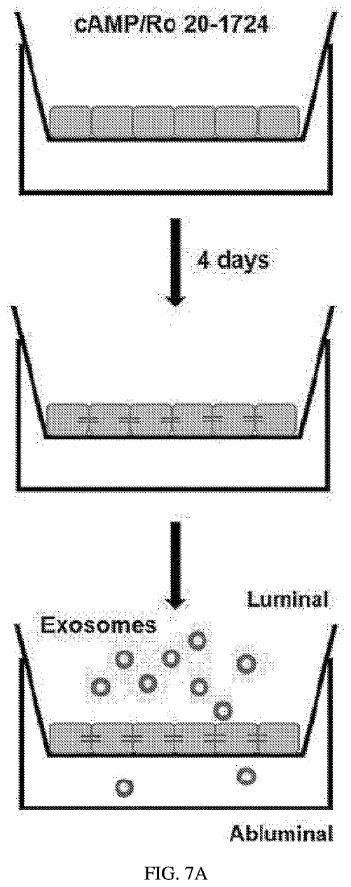

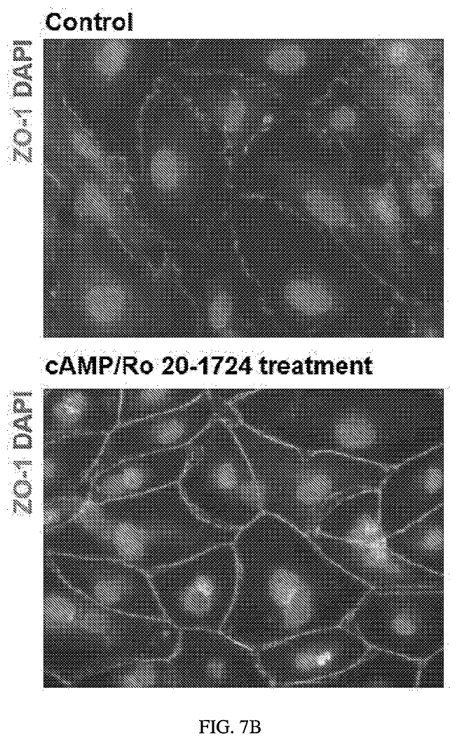

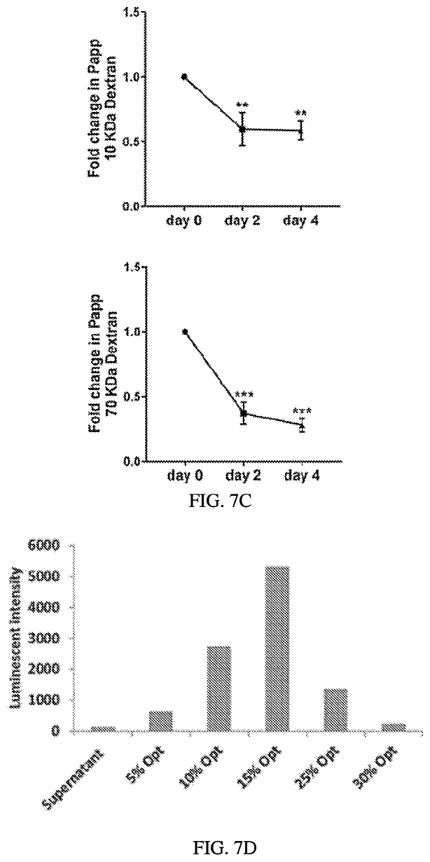

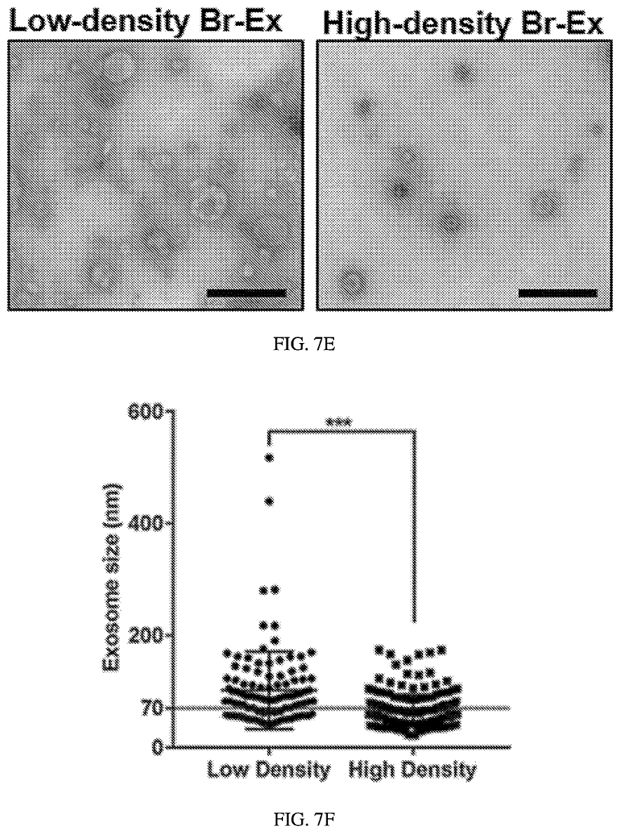

To gain insight into the mechanism(s) by which Br-EVs breach the brain endothelium, an in vitro static BBB model was developed. Primary human brain endothelial cells rapidly lose their junctional features in culture and therefore cannot recapitulate the integrity of the in vivo BBB²¹. It has been shown that with an increase in internal cAMP, these cells can regain their barrier characteristics²². Accordingly, in the BBB model disclosed herein, human brain endothelial cells cultured on Transwell® filters were treated with a combination of CPT-cAMP and Ro 20-1724, an inhibitor of cAMP degradation²², to enhance the expression of junctional proteins such as ZO-1 (FIG. 7A-7B). This treatment resulted in an approximately 50% and 80% reduction in the permeability coefficient of the endothelial monolayer to 10 KDa Alexa 647 (post-treatment Papp 2.15E-6±4.964E-07 cm/s) and 70 KDa fluorescein isothiocyanate (FITC) dextran (post-treatment Papp 1.933E-07±6.26E-08 cm/s), respectively (FIG. 7C).

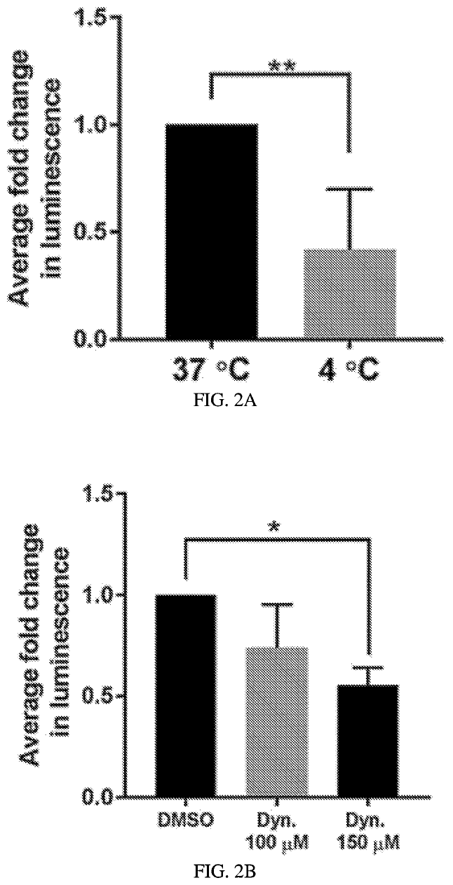

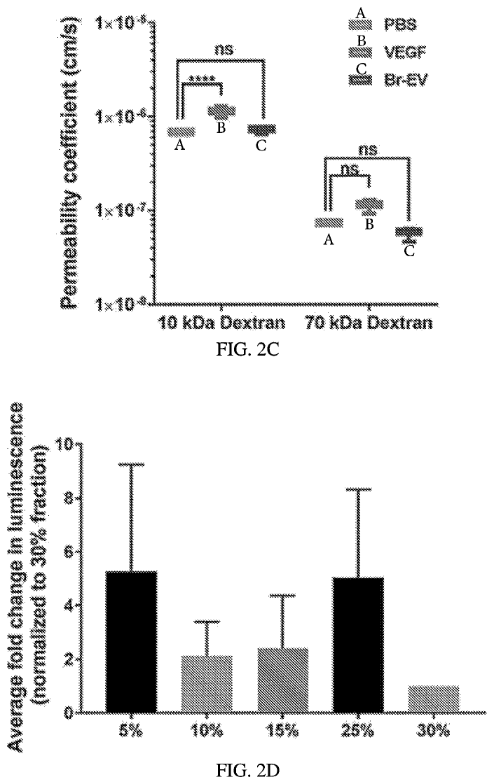

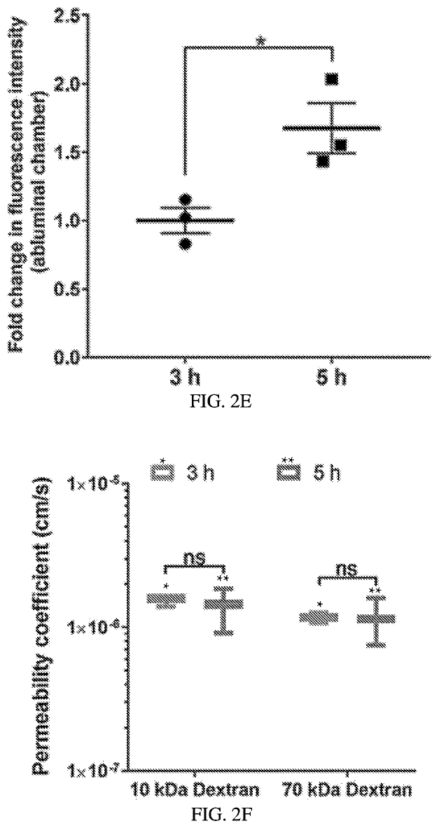

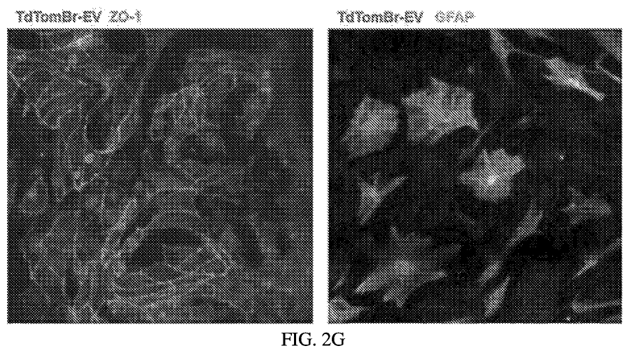

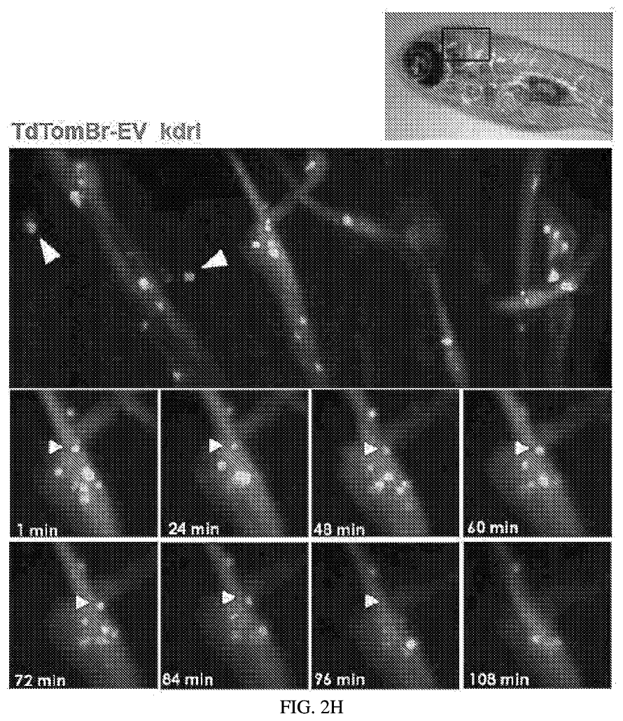

To determine whether the transfer of EVs across the brain endothelial monolayer is through an active or passive mechanism, Gaussia luciferase-labeled Br-EVs were incubated with brain endothelial cells in the luminal (top) chamber of the Transwell® filters for 2 hours at 37° C. or 4° C. The amount of luminescent signal detected in the abluminal (lower) chamber was significantly lower when the filters were incubated at 4° C. (FIG. 2A), suggesting that a mechanism that is active in physiological conditions is involved in the transport of Br-EVs across the brain endothelial monolayer. Moreover, treatment of cells with Dynasore, an inhibitor of endocytosis²³, also resulted in a dose-dependent decrease in the abluminal signal (FIG. 2B). The permeability of the barrier to 10 KDa and 70 KDa dextran was not changed by Br-EVs during this incubation (FIG. 2C). To verify that the source of the detected signal in the abluminal chamber was the luciferase associated with intact EVs as opposed to free luciferase, the media from the lower chamber was ultracentrifuged on an iodixanol OPTIPREP™ density gradient. As a positive control, Gaussia luciferase-labeled Br-EVs were added directly to the top of a gradient for ultracentrifugation. Consistent with previous findings, in the positive control group, luminescent signal was detected at low- and high-density fractions, corresponding to EV density of 1.105-1.184 g/ml (FIG. 7D). In the fractions collected from the media in the lower chamber, luminescent signal was detected in the high-density fraction, corresponding to EV density of 1.184 g/ml (FIG. 2D), confirming the EV source of the signal. It should be noted that some signal was also detected in the supernatant, indicative of free luciferase that could have been released during the processing and degradation of a subpopulation of EVs. Electron microscopy analysis of the low- and high-density fractions of EVs showed that high-density Br-EVs generally had a smaller size with 68% being below 70 nm, whereas this percentage was 34% in the low-density EVs (FIG. 7E-7F). This finding suggests that a high-density subpopulation of EVs that are smaller in size can undergo a transcellular transport. Taken together, these findings in a static in vitro BBB model, suggested that the transport of Br-EVs across the brain endothelial monolayer relies on an active endocytic mechanism, indicative of transcytosis.

To confirm that the static incubation of EVs with endothelial cells does not act as a confounding factor on the mechanism of EV transport, these findings were verified in a microfluidic organ-on-a-chip model of the BBB (BBB chip)²⁴. The BBB chip is a 2-channel microfluidic culture device that contains of a vascular channel lined by induced pluripotent stem cell-derived human microvascular endothelial cells, which is separated by a porous extracellular matrix-coated membrane from an abluminal channel containing primary human astrocytes and pericytes²⁴. TdTom-Br-EVs were flowed through the lumen of the vascular channel for 5 hours. Fluorescent signal was detected in the abluminal chamber at 3 hours and increased significantly over time (FIG. 2E), under conditions in which permeability of the barrier to 10 KDa and 70 KDa dextran did not change (FIG. 2F). Moreover, fluorescence microscopic analysis revealed the presence of Br-EVs that were taken up by astrocytes in the abluminal chamber (FIG. 2G). Overall, these findings demonstrated that Br-EVs can interact with endothelial cells under flow conditions and continuously cross the endothelial monolayer through transcytosis.

Next, the transcytosis of Br-EVs was explored in vivo, using a zebrafish model. Zebrafish develop a mature BBB at 3 days post-fertilization (dpf) and serve as a suitable model for BBB studies²⁵. An intracardiac injection of TdTom-Br-EVs in 6-7-dpf Tg (kdrl:GFP) zebrafish embryos was conducted and the distribution of Br-EVs in the brain was monitored through live imaging. At the time of imaging, Br-EVs were taken up by a number of cells within the brain parenchyma, demonstrating their ability to go beyond the BBB in vivo (FIG. 2H). Moreover, with time-lapse imaging, movement of EV-containing endocytic vesicles within endothelial cells could be observed. As shown in FIG. 2H, a number of these vesicles moved toward the plasma membrane and fused with the membrane, suggestive of a transcytosis process. The integrity of the BBB remained intact throughout the duration of these experiments, as treatment with Br-EVs did not increase the permeability of the BBB to 10 KDa and 70 KDa dextran in zebrafish (FIG. 2I-2J). Together, these in vitro and in vivo findings demonstrate that a subpopulation of Br-EVs can breach the brain endothelial barrier through transcytosis, in a manner that does not compromise junctional permeability.

### Br-EV Transcytosis Involves Caveolin-Independent Endocytosis, Recycling Endosomes and Basolateral SNAREs

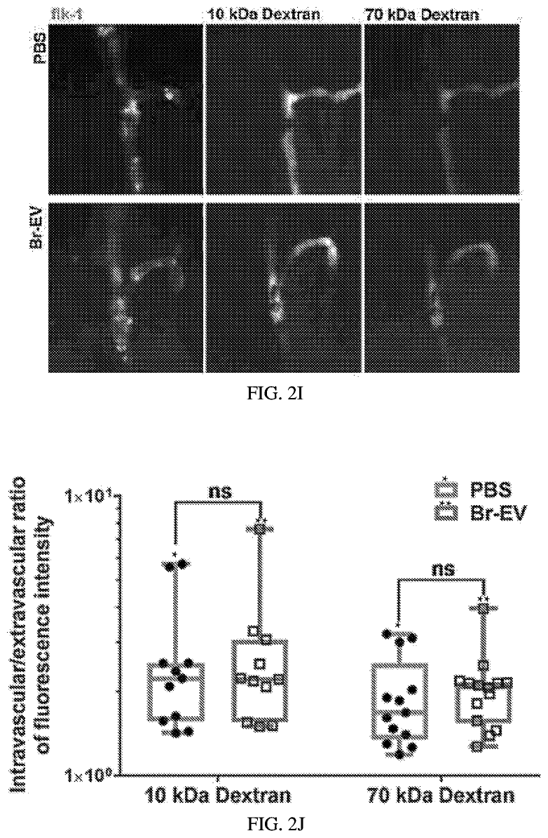

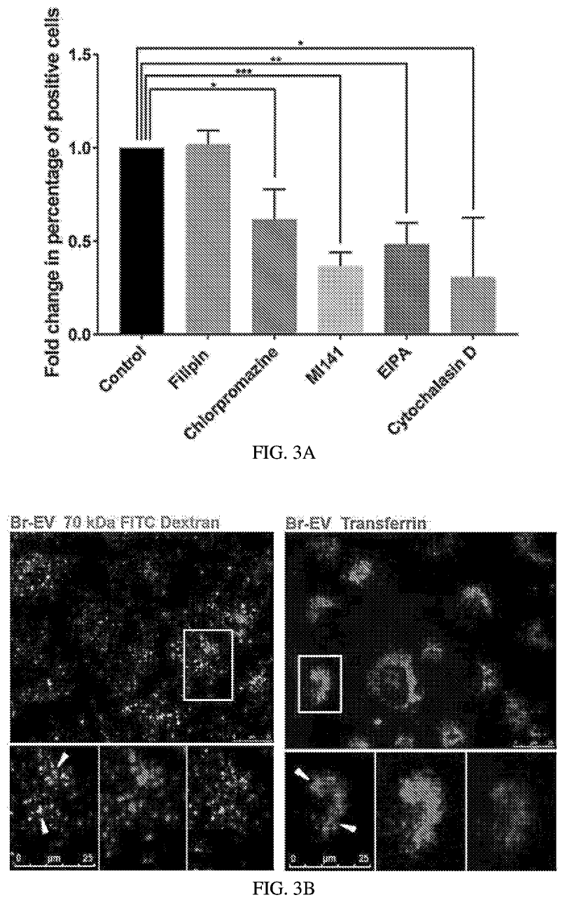

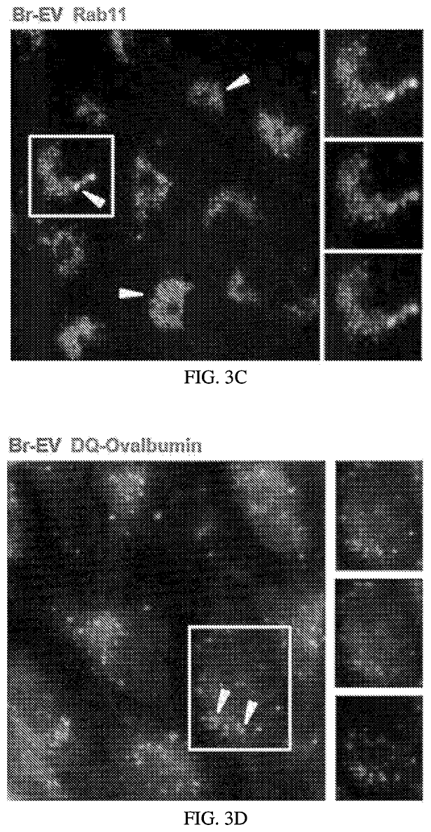

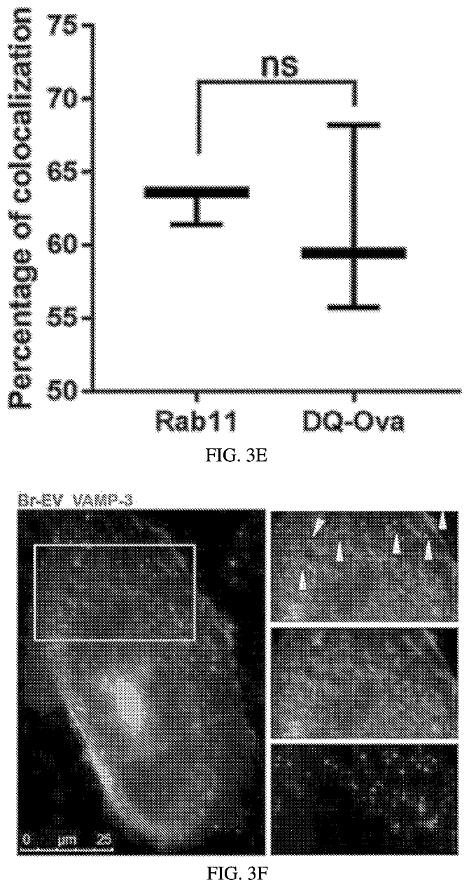

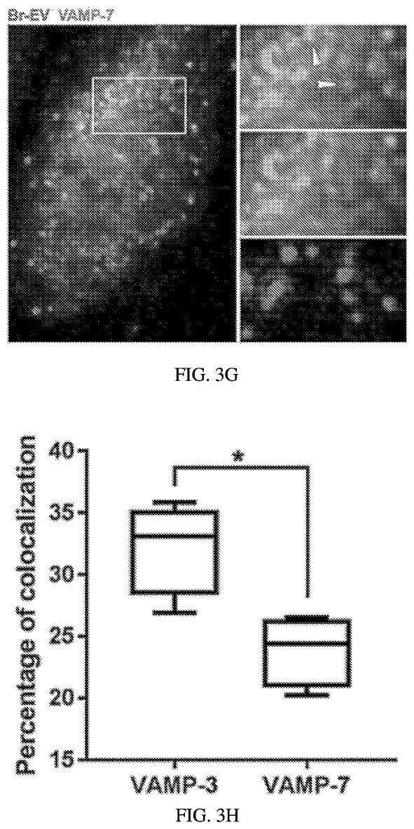

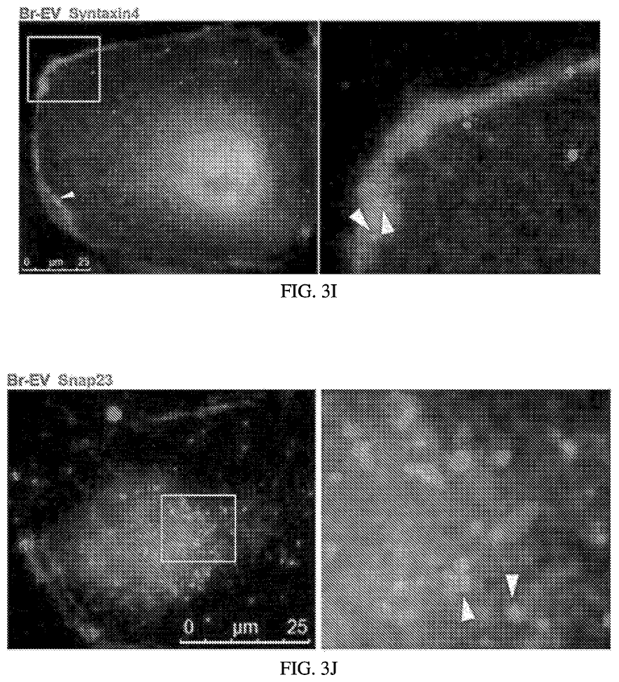

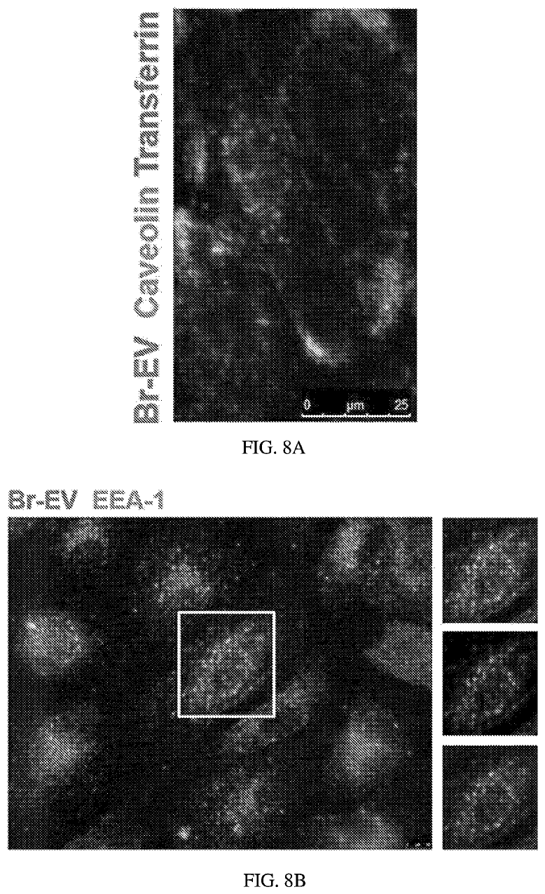

The mechanistic details of Br-EV transport was further explored through brain endothelial cells by focusing on the three major steps of transcytosis: 1) endocytosis through the apical (luminal) membrane of brain endothelial cells, 2) transfer through the endocytic pathway, and 3) release into the extracellular environment from the basolateral (abluminal) membrane. To evaluate the mechanism(s) of uptake, brain endothelial cells were pretreated with chemical inhibitors of the different endocytosis pathways and measured the uptake of TdTom-Br-EVs via flow cytometry. Inhibition of clathrin-dependent endocytosis by chlorpromazine 26 and Cdc42/Rac1 GTPase inhibitor, ML141²⁷, significantly decreased the uptake of Br-EVs (FIG. 3A). Inhibition of macropinocytosis by 5-(N-Ethyl-N-isopropyl) amiloride (EIPA) and cytochalasin D²⁶ also lead to a significant decrease in the uptake of Br-EVs. Further confirming these findings, Br-EVs partially colocalized with transferrin and 70 KDa dextran, markers of clathrin-dependent endocytosis²⁸ and macropinocytosis²⁹, respectively (FIG. 3B). Filipin, an inhibitor of caveolin-dependent endocytosis²⁶, showed no effect on Br-EVs uptake by endothelial cells (FIG. 3A). A lack of co-localization of Br-EVs with caveolin also indicated that caveolin-dependent endocytosis is not involved in the uptake of Br-EVs by brain endothelial cells (FIG. 8A).

To study the second step of transcytosis, the intracellular trafficking of Br-EVs was evaluated. Upon endocytosis, the majority of molecules are transferred to early endosomes, where they are sorted to different routes. Molecules sorted into late endosomes are eventually transferred to lysosomes for degradation, whereas molecules sorted into recycling endosomes will be transferred to the plasma membrane³⁰. Rab11 recycling endosomes have been shown to be involved in the transcytosis of macromolecules^{31,32} As expected, following endocytosis, Br-EVs colocalized with EEA1, a marker of early endosomes³³ (FIG. 8B). To examine whether Br-EVs can proceed through the recycling route in the endocytic pathway for transcytosis, the co-localization of Br-EVs with rab11, a marker of recycling endosomes³³, was evaluated. 62.9±1.27% of Br-EV-containing vesicles colocalized with rab11 in the perinuclear region (FIG. 3C, FIG. 3E). The trafficking of Br-EVs to the degradation route was also evaluated using BODIPY®-conjugated DQ-Ovalbumin as a marker of endo-lysosomal structures^{34, 35}. DQ-Ovalbumin is a self-quenched marker that only fluoresces upon the release of quenching following degradation in late endosomal structures and lysosomes³⁵. As expected, colocalization of a subset of Br-EVs (61.1±6.4%) with DQ-Ovalbumin was also observed in the perinuclear region (FIG. 3D-3E). These findings demonstrate that different subpopulations of Br-EVs can be sorted into different endocytic pathways that would lead to their recycling/transcytosis or degradation.

Finally, the interaction of Br-EV-containing endocytic vesicles with the basolateral membrane was evaluated. Soluble NSF Attachment Protein Receptors (SNARE) are known to be involved in vesicle fusion with the target membrane and include vesicle SNAREs (v-SNAREs) and target SNAREs (t-SNAREs)³⁶. Among the different types of v-SNAREs, vesicle associated membrane protein (VAMP)-3 is associated with recycling endosomes and is involved in exocytosis, whereas VAMP-7 is involved in the fusion of late endosomes with lysosomes³⁷. Microscopy analyses demonstrated that Br-EV-containing vesicles colocalized with both VAMP-3 and VAMP-7 (FIG. 3F-3H). However, colocalization with VAMP-3 was significantly higher than VAMP-7, suggesting that recycling/transcytosis of Br-EVs was a prominent event in this case. The fusion of recycling endosomes with the basolateral plasma membrane occurs through the interaction of VAMP-3 with SNAP23/Syntaxin 4, a t-SNARE complex localized on the basolateral membrane^{38, 39}. Here, Br-EV-containing vesicles colocalized with both SNAP23 and Syntaxin4 (FIG. 3I-3J), demonstrating the involvement of these SNARE complexes in the fusion of these vesicles with the basolateral membrane. Taken together, these findings demonstrate that while a subpopulation of Br-EVs are sorted into late endosomes for degradation, a large subset of these EVs can be sorted into rab11+ recycling endosomes, which could lead to the VAMP3/Snap23/Syntaxin4-dependent release of these EVs at the basolateral membrane.

### Br-EVs Decrease the Astrocyte Expression of TIMP-2

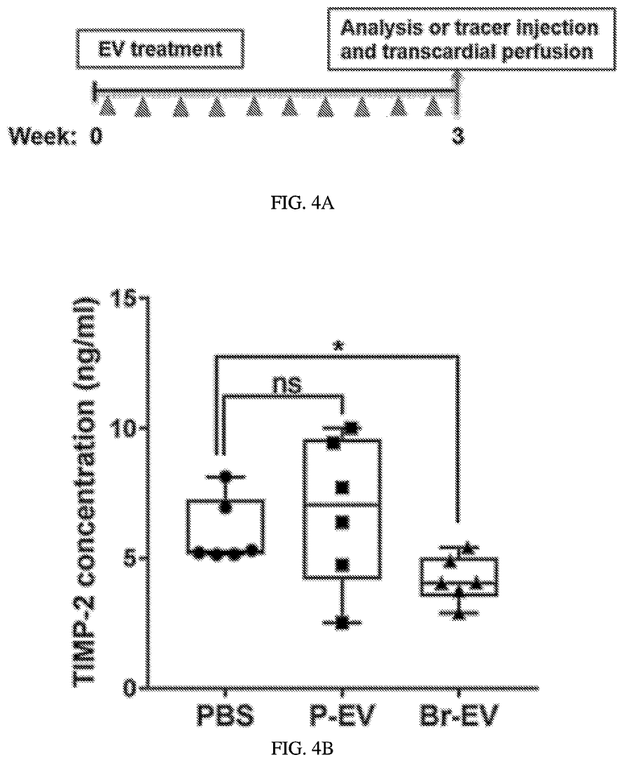

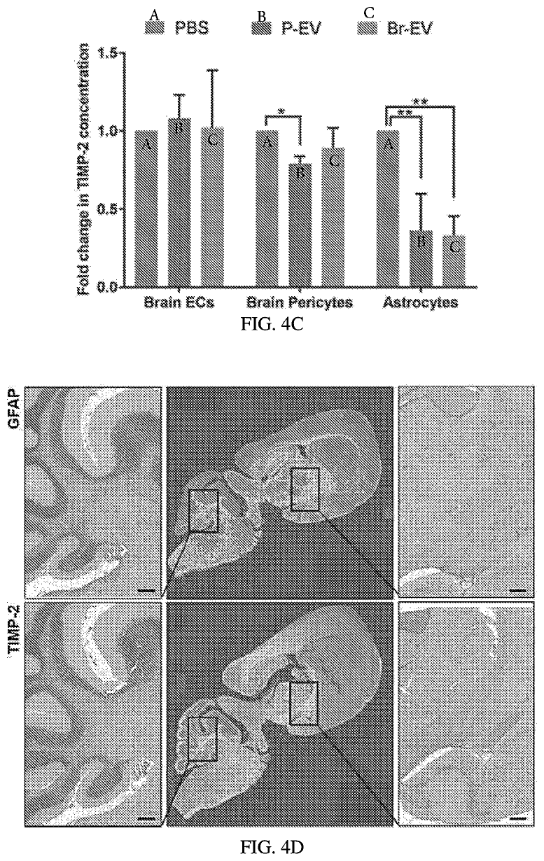

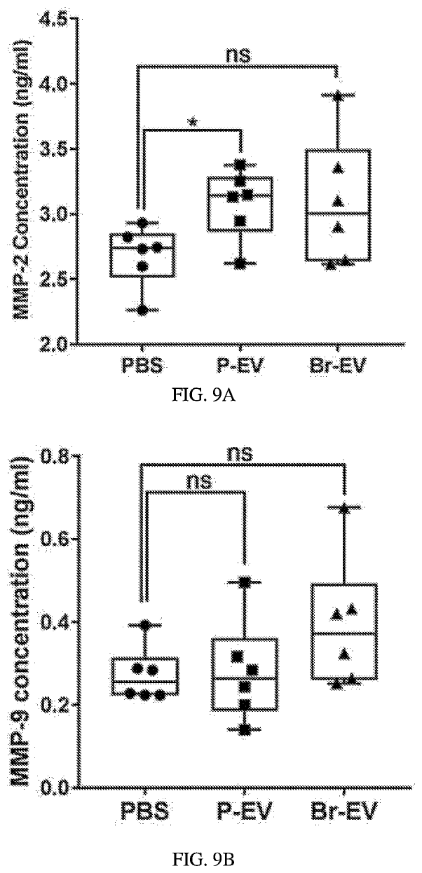

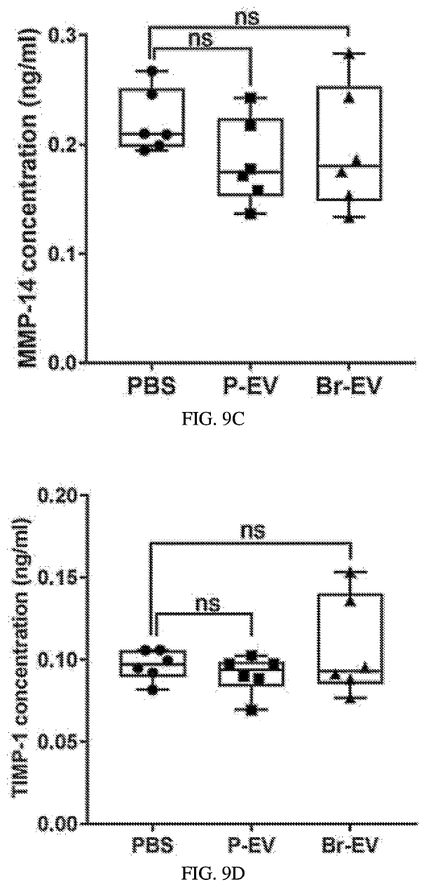

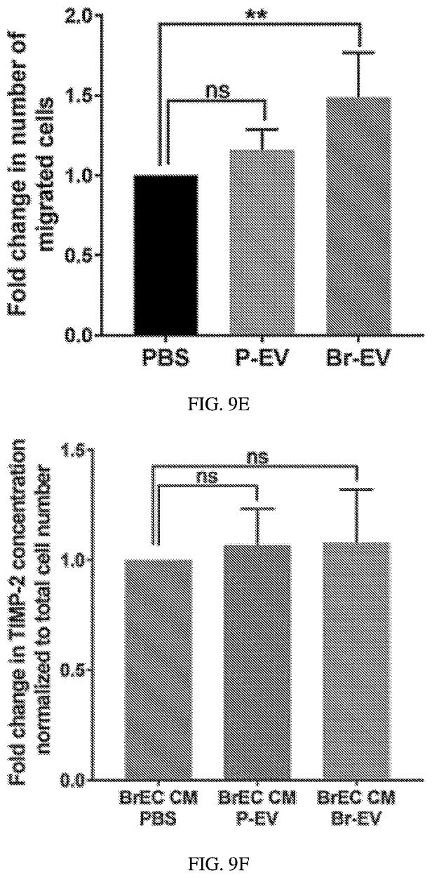

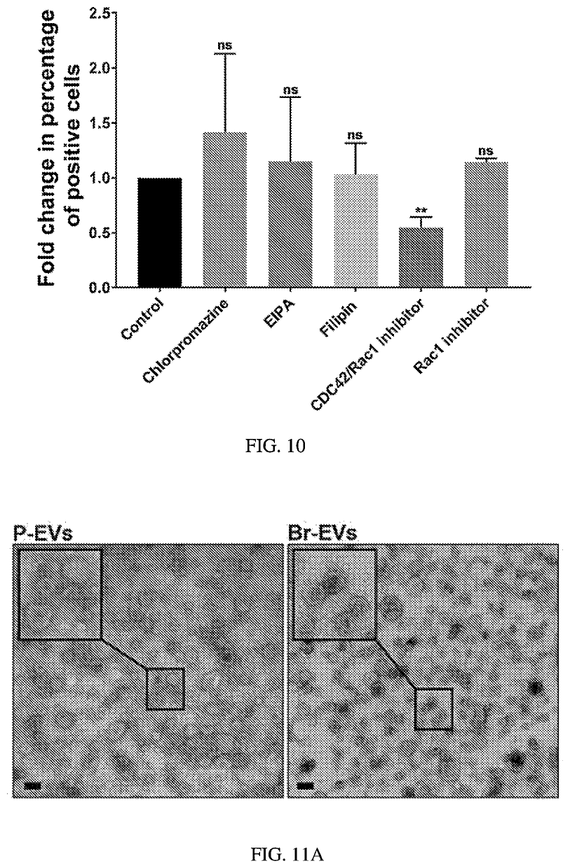

To determine whether the transcellular transport of Br-EVs across the BBB has functional consequences, the effect(s) of Br-EVs on the behavior of the BBB cells were evaluated. It was hypothesized that upon transcytosis, Br-EVs can change the behavior of the BBB cells on the abluminal side (i.e. astrocytes and pericytes) to prepare a microenvironment supportive of tumor cell growth. It is widely acknowledged that matrix metalloproteinases (MMPs) and their endogenous inhibitors, the tissue inhibitors of MMPs (TIMPs) can contribute to tumor progression and metastasis⁴⁰⁻⁴³. Through modulating the ECM, these enzymes can trigger different signaling pathways and promote the tumor-supporting microenvironment^{5, 44}. Different MMPs including MMP-2 and MMP-9 are known to be involved in the preparation of a niche for tumor cell growth in the brain⁴⁵⁻⁴⁹. Moreover, the importance of astrocyte-derived MMPs and TIMPs in brain metastasis has been previously demonstrated⁵⁰. Accordingly, the ability of Br-EVs to alter the expression of MMPs and TIMPs in astrocytes and/or pericytes to facilitate brain metastasis was tested. Mice were treated with P-EVs and Br-EVs as above and sacrificed following 10 EV injections to analyze the brain tissue (FIG. 4A). Using mouse-specific enzyme-linked immunosorbent assays (ELISA), the expression of a number of MMPs and TIMPs that are known to be expressed in brain tissue⁵¹ were analyzed, including MMP-2, MMP-9, MMP-14, TIMP-1, and TIMP-2 in mouse brain tissue homogenates (FIG. 4B, FIG. 9A-9D). It was found that TIMP-2, the endogenous inhibitor of MMP-2 activity 52, was exclusively and significantly decreased by brain metastasis-promoting Br-EVs but not P-EVs (FIG. 4B).

The abluminal cells of the BBB were investigated to determine whether they could be the source of the Br-EV-driven decrease in brain TIMP-2. Human BBB cells were treated with P-EVs and Br-EVs in vitro and evaluated TIMP-2 expression using a human-specific TIMP-2 ELISA. EV treatment did not affect the expression of TIMP-2 in brain endothelial cells (FIG. 4C). Both P-EVs and Br-EVs significantly decreased the expression of TIMP-2 in astrocytes (FIG. 4C). Moreover, Br-EVs were able to increase the migration of astrocytes, which was consistent with the observed decrease in TIMP-2 expression and a subsequent increase in ECM modulation (FIG. 9E). This finding was consistent with the hypothesis that Br-EVs can change the behavior of abluminal cells of the BBB. To rule out the possibility that the decreased astrocyte TIMP-2 might be an indirect effect of Br-EVs acting through brain endothelial cells, conditioned media was prepared by treating brain endothelial cells with EVs or PBS. Astrocytes were incubated with the endothelial cell conditioned media for 48 hours, followed by the media exchange and subsequent analysis of the astrocyte conditioned media. No difference in TIMP-2 levels were found in conditioned media from astrocytes that were incubated with PBS-, P-EV-, or Br-EV-treated endothelial cell conditioned media (FIG. 9F). In addition, consecutive brain tissue sections were stained for TIMP-2 and the astrocyte marker, GFAP, and found that areas that were rich in astrocytes also had a high expression of TIMP-2, further supporting astrocytes as the major source of TIMP-2 (FIG. 4D).

Next, the observed decrease in astrocyte TIMP-2 levels following Br-EV treatment was tested to determine if it was accompanied by alterations in the permeability of the BBB. No increase in permeability of brain endothelium to 10 KDa- and 70 KDa-dextran was observed following P- or Br-EV treatment (FIG. 4E). This observation suggested that the BBB remained intact during this experiment, supporting the conclusion that the EV-induced decrease in TIMP-2 was a direct effect of transcytosed Br-EVs on astrocytes. Overall, these findings indicate that the transcytosis of Br-EVs and subsequent uptake by astrocytes can have functional consequences, such as suppressed TIMP-2 expression that can lead to the preparation of a microenvironment at the BBB suitable for metastases growth.

Br-EVs Downregulate Endothelial Rab7 to Facilitate their Transport

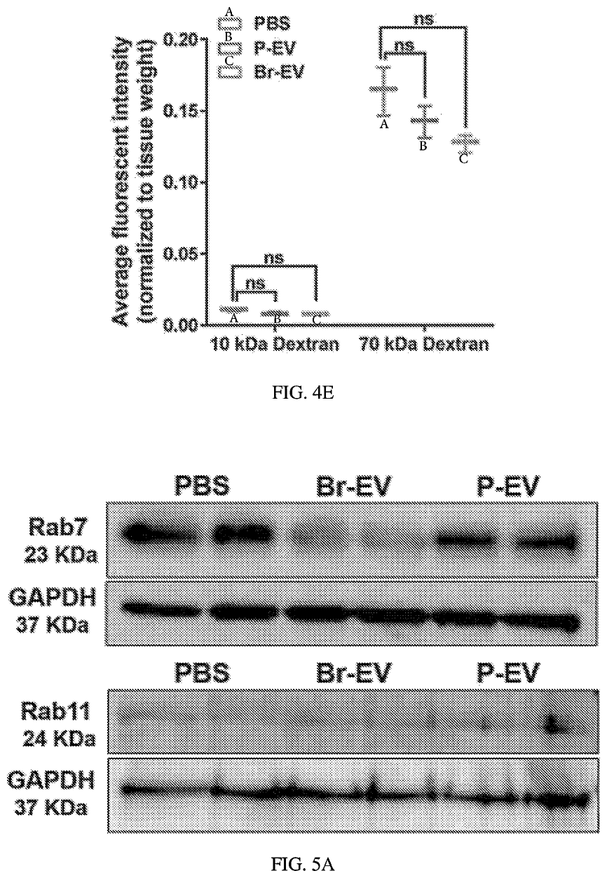

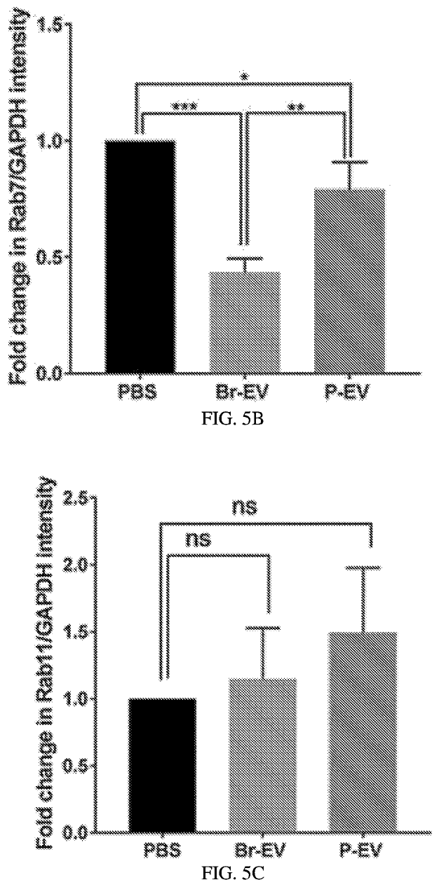

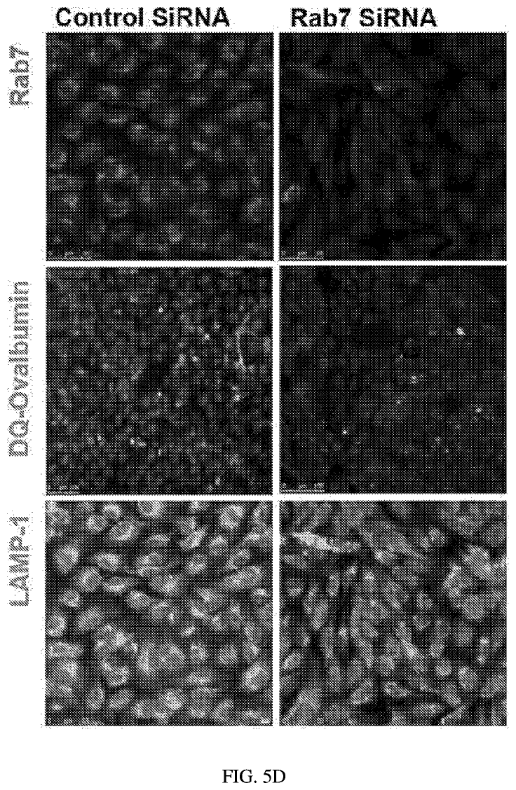

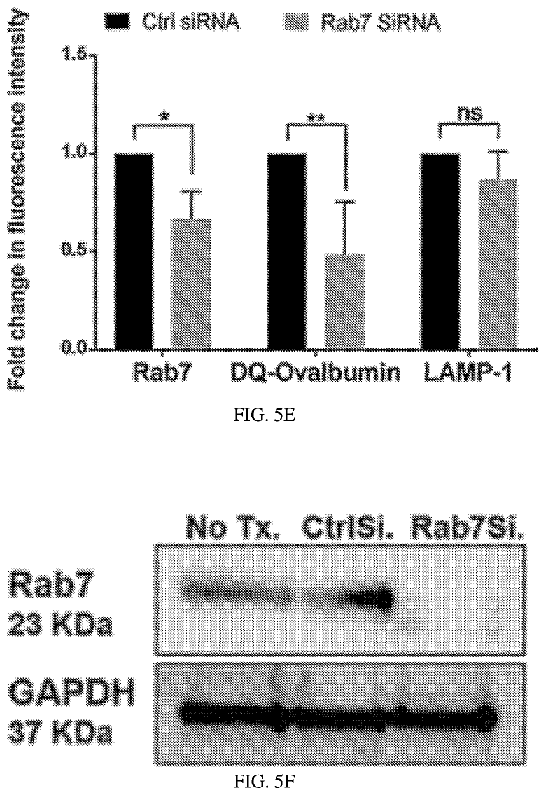

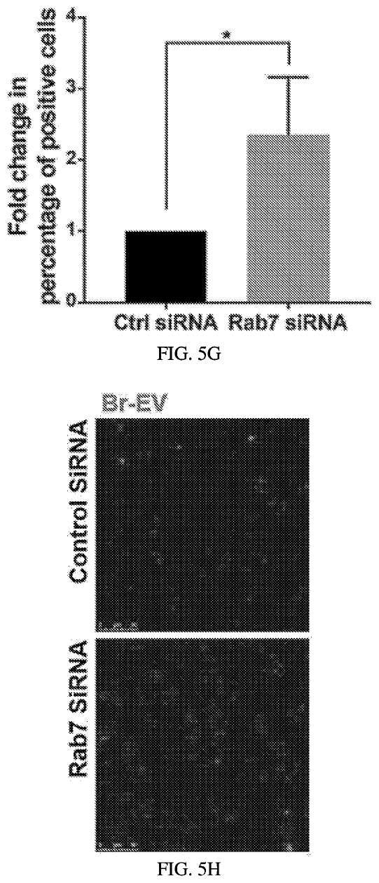

As shown in FIG. 4C, the in vitro experiments indicated that both P-EVs and Br-EVs had the ability to reduce the astrocyte expression of TIMP-2, whereas, surprisingly, only Br-EVs could induce this effect in vivo (FIG. 4B). These findings suggest that the overall transport of Br-EVs across the BBB, which is a prerequisite for their effects on astrocytes, is more efficient compared to P-EVs. To address this possibility, it was hypothesized that Br-EVs can specifically modulate the endocytic pathway in brain endothelial cells to increase their transport efficiency. The effect of EV treatment on the two major routes in the endocytic pathway, degradation and recycling was evaluated. Br-EV treatment of brain endothelial cells significantly decreased the expression of the late endosomal marker, rab7, whereas the expression of rab11, marker of recycling endosomes, was not changed (FIG. 5A-5C). This finding demonstrated that Br-EVs exhibit a unique ability to modulate the degradation pathway in brain endothelial cells.

Rab7 is involved in the transfer of early endosomes to late endosomes and late endosomes to lysosomes⁵³. To determine whether the decrease in endothelial rab7 can lead to a decrease in the transfer of molecules to lysosomes, siRNA was used to knockdown the expression of rab7 in brain endothelial cells. Then, the cells were treated with DQ-Ovalbumin, which fluoresces upon being processed in late endo-lysosomal structures³⁵. Rab7 KD decreased the fluorescent signal from DQ-Ovalbumin, suggesting a decrease in the transfer of this molecule to late endosomes and lysosomes (FIG. 5D-5E). The total number of lysosomes as measured by the lysosomal marker, LAMP1, was not changed by Rab7 KD.

Rab7 can also interact with, and increase, the activity of rac1, a small GTPase protein that acts as a central regulator of actin remodeling⁵⁴⁻⁵⁶ The activation level of Rac1 can control the rate of endocytosis and rab7 can be indirectly involved in this process^{56, 57}. Accordingly, it was hypothesized that the Br-EV-driven decrease in rab7 can indirectly affect the rate of EV endocytosis. Flow cytometry studies demonstrated that rab7 KD significantly increased the uptake of Br-EVs by brain endothelial cells (FIG. 5F-5G). Fluorescent microscopy of Br-EV uptake by endothelial cells demonstrated that the pattern and the size of Br-EVs-containing endosomes were not different between Rab7 and control siRNA-treated cells (FIG. 5H-5I). This result confirmed that increased signal detected by flow cytometry was due to increased uptake of Br-EVs rather than the accumulation of Br-EVs in lysosomal structures.

Taken together, these findings suggest that Br-EVs can increase their transport efficiency across the brain endothelial cells by decreasing the expression of rab7 in brain endothelial cells. This decrease in rab7 expression can eventually increase the uptake of Br-EVs and disrupt the endocytic trafficking into the degradation path.

### Discussion

Homing of tumor-derived EVs to pre-metastatic organs has been described as an early event that leads to the preparation of a pre-metastatic niche for future metastasis⁵. Herein, it is demonstrated for the first time that breast cancer-derived EVs can cross an intact BBB through transcytosis. The mechanistic events that lead to tumor-derived EV transcytosis across the brain endothelium and the functional consequences of this transcellular transport on astrocytes have been identified. These findings expand understanding of the early events in the process of pre-metastatic niche preparation prior to brain metastasis and provide opportunities for development of early diagnostics and therapeutics for brain metastasis.

Using static and flow-based in vitro as well as in vivo models of the BBB, it was demonstrated that Br-EVs undergo a transcellular transport to enter the brain parenchyma, without disrupting the BBB. A leaky BBB, i.e., the blood-tumor-barrier, is one of the hallmarks of brain metastases⁵⁸. It has been reported that the integrity of the BBB is only disrupted following metastases growth, remaining unaffected even during the early micrometastasis stage⁵⁸⁻⁶⁰. These reports along with the findings provided herein suggest that at least during the early stages of pre-metastatic niche preparation, the BBB remains intact. The timing of BBB disruption however, remains a matter of controversy. Recent studies have demonstrated that treatment with breast cancer-derived EVs can increase the permeability of BBB through downregulating ZO-1 expression and modulating actin localization^{12, 13}. Variability in the methodology of EV treatment and evaluation of the permeability partly accounts for such contrasting results. Herein, the early stages of pre-metastatic niche preparation were studied and the ability of EVs to breach the BBB via transcytosis prior to a disruption in the BBB integrity was demonstrated.

The density gradient fractionation studies provided herein suggested that a high-density subpopulation of EVs that are smaller in size have the ability to undergo transcytosis in brain endothelial cells. Previous studies have attempted to isolate EV subpopulations with different densities^{61, 62}. Consistent with the findings provided herein, one study found two distinct subpopulations of EVs with low and high density and showed that the high-density EVs were smaller in size⁶¹. This study also demonstrated that the two subpopulations had distinct protein and RNA profiles. More recently, using an asymmetric flow field-flow fractionation method a subpopulation of extracellular vesicles smaller than 50 nm were isolated and were introduced as exomeres⁶³.

It was demonstrated that through downregulating rab7, Br-EVs can modulate the endocytic pathway in brain endothelial cells to increase the efficiency of their transport. This process occurred through two separate mechanisms. Downregulation of rab7 in brain endothelial cells disrupted the degradation route in the endocytic pathway through decreasing the transfer of molecules to lysosomes. This process might also enable endosomes to switch tracks to the recycling route. A supporting mechanism was described recently in a study that showed that knockdown of the NBEAL2 gene in megakaryocytes can disrupt the transport of fibrinogen to rab7 late endosomes and lysosomes⁶⁴. This disruption of degradation increased the transfer of fibrinogen to rab11 recycling endosomes. Rab7 has also been shown to increase the activity of rac1⁵⁶. Increased rac1 activity has been associated with an increase in the rate of macropinocytosis⁵⁶ and a decrease in clathrin-mediated uptake of molecules such as epidermal growth factor and transferrin⁵⁷. In the present disclosure, downregulation of rab7 in brain endothelial cells significantly increased the uptake of Br-EVs.

In summary, the present disclosure has identified transcytosis as the mechanism by which breast cancer-derived EVs can breach the BBB. The studies provided herein indicate that EVs derived from a brain-seeking subpopulation of breast cancer cells can exclusively modify the physiological regulation of the BBB at multiple levels to promote metastasis development in the brain microenvironment. These findings provide new opportunities for early detection and therapeutic intervention in brain metastasis.

Moreover, the present disclosure further exploits the process to develop efficient drug delivery approaches for a variety of brain and CNS disorders including, but not limited to, brain malignancies and neurodegenerative diseases.

### Methods

### Cell Lines and Cell Culture

Human breast cancer cell line MDA-MB-231 was purchased from American Type Culture Collection (ATCC® HTB-26™, VA, USA). The brain-seeking (MDA-231Br) variant of the breast cancer cell line MDA-MB-231 was a gift from Dr. T. Yoneda, Indiana University¹⁸. Primary human brain microvascular endothelial cells, human astrocytes, and human brain vascular pericytes were purchased from Cell Systems Co. (Cat #ACBRI 376, Kirkland, Wash.), Thermo Fisher Scientific Inc. (Cat #N7805100), and ScienCell Research Laboratories (Cat #1200, Carlsbad, Calif., USA), respectively. Breast cancer cells were cultured in Dulbecco's Modified Eagle's medium (DMEM, Cat #11885084, Thermo Fisher Scientific Inc.) supplemented with 10% fetal bovine serum (FBS, Cat #S11150, Atlanta Biologicals™, Atlanta, Ga., USA) and 1% Penicillin-Streptomycin (10,000 U/mL) (Cat #15140148, Thermo Fisher Scientific Inc.). For extracellular vesicle (EV) isolation, breast cancer cells were cultured in DMEM supplemented with 10% EV-depleted FBS. EV-depleted FBS-containing medium was prepared by 18-h ultracentrifugation of media containing 40% FBS at 100,000×g at 4° C.⁶⁵. The EV-depleted media was then diluted to contain 10% EV-depleted FBS. Human brain endothelial cells were cultured with endothelial cell growth medium (EGM™-2MV, Cat #CC-3202, Lonza Inc., ME, USA). Human astrocytes and brain pericytes were cultured according to the manufacturer's instructions. All cells were maintained in a 37° C. humidified incubator with 5% CO2. All cultures were routinely monitored for mycoplasma contamination using the MycoAlert™ PLUS Mycoplasma Detection Kit (Cat #LT07-710, Lonza Inc.).

### EV Isolation and Characterization

Conditioned media was collected from breast cancer cell cultures after 24 hours of incubation in EV-depleted media. Conditioned media was only used for EV collection if cell viability was >95%. EVs were isolated using a sequential centrifugation technique¹⁹. Briefly, conditioned media was centrifuged at 400×g for 10 min, 2000×g for 15 min, and 15000×g for 30 minutes at 4° C. (Sorvall® RC-5B centrifuge, Thermo Fisher Scientific Inc.) to remove dead cells, debris, and larger microvesicles. The supernatant subsequently underwent a round of ultracentrifugation at 100000×g for 90 minutes at 4° C. (Optima XE-90 Ultracentrifuge, Beckman Coulter Life Sciences) followed by a round of wash at 100000×g for another 90 minutes. The final pellet was resuspended in PBS for characterization and experiments.

EV preparations were characterized according to the guidelines of the International Society for Extracellular Vesicles²⁰. EV size and concentration was measured by nanoparticle tracking analysis (NanoSight NS300, Malvern Instruments, UK). The presence of EV markers CD9, CD63, and Alix and the absence of a golgi marker (GM130) as a negative control was evaluated by western blot. The shape of the EVs was evaluated by electron microscopy. To this end, EV samples were adsorbed to a formvar/carbon-coated grid and stained with uranyl formate. The grids were imaged using a JEOL 1200EX Transmission electron microscope and images were taken with an AMT 2k CCD camera. EV density was measured using an OPTIPREP™ Gradient ultracentrifugation technique. Briefly, EVs were suspended in OPTIPREP™ to prepare a 5% concentration. The EV-containing OPTIPREP™ was then layered on top of a gradient consisting of 10%, 25%, and 30% OPTIPREP™. Gradients were centrifuged at 100,000×g for 4 hours at 4° C. All fractions were collected and were either used directly for luciferase assay or were further diluted in PBS (1:25) and centrifuged at 100,000×g for 90 minutes at 4° C. The pellets were resuspended in PBS and were used for western blot analyses.

### EV Labeling

231P and 231Br breast cancer cells were transduced with lentiviral vectors to express palmitoylated TdTomato (PalmtdTomato)⁶⁶ or membrane-bound Gaussia luciferase⁶⁷. Both DNA constructs were gifts of Dr. X. Breakefield, Massachusetts General Hospital (MGH), and the lentivirus vectors were made at the MGH Vector Core, Boston, Mass. EVs were isolated from stable clones as described above. The presence of labels on EVs was confirmed via fluorescent microscopy for tdTomato and via luciferase assay for Gaussia luciferase. Briefly, a 20 μM concentration Gaussia luciferase substrate, native Coelenterazine (Prolume Ltd. Cat #303), was prepared and incubated for 30 minutes at room temperature. 50 μl of the substrate was added to each well containing the samples and luminescence intensity was measured immediately using a SpectraMax M2 plate reader (Molecular Devices, Inc.).

### In Vitro EV Uptake Studies

To evaluate the uptake of EVs by brain ECs, astrocytes and pericytes, cells grown to confluence in 96-well plates were incubated with 2×10⁹ particle/well of tdTomato P- and Br-EVs. After 2 hours of incubation, cells were washed for 3 times and were fixed for imaging. Images from four different fields were taken using a Zeiss Fluorescent microscope and the level of fluorescence intensity was analyzed using the ImageJ software. To eliminate the confounding effect of cell size on the uptake level, fluorescent intensity for tdTom-EVs was measured per unit of cell surface area. For endocytosis inhibition studies, brain ECs cultured in 12-well plates were treated with chlorpromazine hydrochloride (Millipore Sigma, Cat #C8138, 20 μM), ML141 (100 μM, Millipore Sigma, Cat #217708), 5-(N-Ethyl-N-isopropyl) amiloride (EIPA) (100 μM, Tocris, Cat #3378), cytochalasin D (500 nM, Tocris, Cat #1233), and filipin III (10 μM, Millipore Sigma, Cat #F4767) for 30 minutes prior to addition of TdTom-Br-EVs (10¹⁰ particle/well). Following 3 hours of incubation with EVs, cells were washed and EV uptake was measured by flow cytometry using a BD FACSCalibur flow cytometer (BD Biosciences, San Jose, Calif.).

### In Vitro EV Functional Studies

To evaluate the direct effect of EVs on the expression profile of BBB cells, primary human brain ECs, astrocytes, and pericytes cultured in 6-well plates were treated with P- or Br-EVs every day for 3 days (5 μg EVs per treatment). Following this continuous treatment, cell lysates and conditioned media were collected for downstream analyses. The amount of TIMP-2 was measured in conditioned media using a human TIMP-2 ELISA (R&D Systems Inc. Cat #DTM200) according to the manufacturer's protocol. The expression of rab7 and rab11 were evaluated by western blotting.

For astrocyte migration studies following continuous EV treatment, astrocytes were trypsinized and were plated in Transwell® filters (8 μm pores, Costar Transwell® Assay; Corning Inc., Corning N.Y.) in astrocyte serum-free media (2×10⁴ cells in 100 μl media per filter). Filters were placed in 24-well plates containing 600 μl of complete astrocyte media. After 16 h, cells were fixed and stained with DAPI. Cells attached to the top of the filter were removed. Membranes were separated from the filters and were mounted on glass slides. The number of cells on the bottom of the filters were counted using a Zeiss Axiocam fluorescent microscope at 200× magnification (4 fields/filter, n=2 filters per treatment).

To assess any indirect effects of EVs on astrocytes, brain ECs were initially treated with P- or Br-EVs as described above. After 3 days of treatment, EC conditioned media was collected. Astrocytes cultured in 6-well plates were incubated with EC conditioned media for 48 hours, following which the cells were serum starved overnight and the astrocyte conditioned media was collected for analysis. TIMP-2 levels were measured using a human TIMP-2 ELISA as described above.

### In Vitro Transcytosis Studies

### Static BBB Model

TRANSWELL™ filters (0.4 μm pore polycarbonate membrane inserts, Cat #C3472, CORNING™ Inc., MA) were coated with 50 μg/ml human plasma fibronectin (Cat #FC010, EMD Millipore) for 1 hour at 37° C. Brain ECs were cultured on filters (25×10³ cells per filter) and incubated for 48 hours to reach full confluency. At this time, the cells were fed with endothelial growth media supplemented with 8-(4-Chlorophenylthio) adenosine 3′,5′-cyclic monophosphate (8-CPT-cAMP, 50 nM, Cat #ab120424, Abcam) and 4-(3-Butoxy-4-methoxybenzyl)-2-imidazolidinone Ro 20-1724 (17.5 nM, Cat #CAS 29925-17-5, Santa Cruz Biotechnology). To determine the integrity of the endothelial monolayer, 10 KDa Dextran, ALEXA FLUOR™ 647 (Cat 3 D22914, ThermoFisher Scientific) and 70 KDa Fluorescein isothiocyanate (FITC)—dextran (Cat #FD70S, Sigma Aldrich) were added to the upper chamber of the TRANSWELL™ filters (100 μg/ml) and the fluorescence intensity in the media of the lower chamber was measured after 20 minutes using a SpectraMax M2 plate reader (Molecular Devices, Inc.). The apparent permeability coefficient was calculated for each tracer, as described previously²⁴.

To evaluate the transport of EVs using this model, Gaussia luciferase-labeled Br-EVs were added to the upper chamber (8×10⁹ particles in 100 μl of media). To evaluate the effect of temperature, filters were incubated at either 4° C. or 37° C. To evaluate the effect of endocytosis, filters were pretreated with Dynasore hydrate (Millipore Sigma, Cat #D7693) for 30 minutes prior to adding the EVs and then incubated at 37° C. The media from the lower chamber was collected after 2 hours and luminescence intensity was measured as described before. To evaluate the intactness of EVs in the lower chamber media, the media collected from the lower chamber or Gaussia luciferase-labeled Br-EVs (as positive control) were run over an OPTIPREP™ density gradient as described previously. After 4 hours of ultracentrifugation, different density fractions were isolated and luminescence intensity was measured for each fraction. To evaluate the effect of EVs on the integrity of the brain EC monolayer, filters were treated with either Br-EVs (8×10⁹ particles per filter) or with recombinant human vascular endothelial growth factor (R&D Systems Inc., Cat #293-VE-010) as a positive control⁴¹. After 2 hours of incubation, the permeability of the filters to 10 KDa ALEXA FLUOR™ 647 Dextran and 70 KDa FITC-dextran was measured as described above.

### Flow-Based BBB Chip

Microfluidic BBB chips were prepared as reported previously²⁴. TdTom-Br-EVs with a concentration of 10¹¹ particles/ml (for transcytosis studies) or a combination of unlabeled Br-EVs (1011 particles/ml), 10 KDa Dextran, ALEXA FLUOR™ 647 (100 μg/ml) and 70 KDa FITC-dextran (100 μg/ml) (for permeability studies) were added to the lumen of the vascular channel at a flow rate of 100 μl/hour for 5 hours. Media from outlets of both the vascular and abluminal channels were collected separately at 3 hours and 5 hours and fluorescence intensity was evaluated using a BioTek plate reader and the Synergy Neo GENS 2.09 software. Apparent permeability of the TdTom-Br-EVs and Dextran tracers under flow conditions were calculated using a previously reported formula²⁴.

### In Vitro Colocalization Studies

Human brain endothelial cells were cultured on fibronectin-coated glass-bottom microslides (Ibidi, Cat #80827). Confluent cells were used in these studies. Cells were co-incubated with TdTom-Br-EVs (8×10⁹ particles/well) and 70 KDa FITC-dextran (0.5 mg/ml), ALEXA FLUOR™ 647-conjugated transferrin (50 μg/ml, Thermo Fisher Scientific, Cat #T23366), or DQ™ Ovalbumin (200 μg/ml, Thermo Fisher Scientific, Cat #D12053) for 30 minutes. Subsequently, cells were washed with PBS, 4 times and fixed with 4% formaldehyde for 10 minutes. For evaluation of co-localization with EEA1 and caveolin-1 (15 minute incubation), or SNARE complexes (30 minute incubation) cells were incubated with TdTom-Br-EVs (8×10⁹ particles/well) and then washed and fixed for staining with anti-EEA1 (1:100, Cell Signaling Technologies, Cat #3288), anti-caveolin-1 (1:100, Cell Signaling Technologies, Cat #3267), anti-VAMP-3 (1:100, Abcam, Cat #ab200657), anti-VAMP-7 (1:100, Cell Signaling Technologies, Cat #13786), anti-syntaxin4 (1:50, R&D Systems Inc., Cat #MAB7894), and anti-snap23 (1:100, R&D Systems Inc., Cat #AF6306) antibodies. For co-localization studies with Rab11, cells were initially transfected with GFP-rab11 plasmid using Lipofectamine 3000 reagent (Thermo Fisher Scientific). GFP-rab11 WT plasmid was a gift from Richard Pagano (Addgene plasmid #12674; http://n2t.net/addgene:12674; RRID:Addgene_12674)⁶⁸. Transfected cells were then cultured on microslides for incubation with TdTom-Br-EVs as described.

Epifluorescence microscopy was performed on a Leica microscope coupled to high-resolution objectives and a Hamamatsu Orca CCD (Japan). To quantify the colocalization of EVs with different markers, at least 10 different fields were evaluated for each experiment. Colocalization with rab11 and DQ™ Ovalbumin was quantified using a plugin for ImageJ developed by Jaskolski et al.⁶⁹. Colocalization with VAMP-3 and VAMP-7 was quantified through manual counting of the percentage of the colocalized EV-containing endosomes.

Rab7 siRNA Studies

Human brain endothelial cells (at 30% confluency) were treated with a pool of Rab7A siRNAs or non-targeting siRNAs (100 nM, Dharmacon, siGENO ME SMARTpool), using DharmaFECT 4 transfection reagent. Experiments were performed 72 hours after transfection. For imaging, cells cultured on microslides, were incubated with DQ™ Ovalbumin (200 μg/ml) or TdTom-Br-EVs (8×10⁹ particles/well) for 30 minutes. Subsequently, cells were washed with PBS, 4 times and fixed for staining with anti-Rab7 antibody (1:100, Abcam, Cat #137029) and anti-LAMP-1 antibody (1:100, Cell Signaling Technologies, Cat #9091). Epifluorescence microscopy was performed as described previously. Using ImageJ, fluorescence intensity was measured in 6 fields for each condition and was normalized to autofluorescence intensity captured from empty microslides. For flow cytometry, transfected cells cultured in 12-well plates were incubated with TdTom-Br-EVs (10¹⁰ particles/well) for 3 hours and cell uptake was quantified through flow cytometry as described above.

### In Vivo Experiments

All animal experiments were conducted in accordance with the Institutional Animal Care and Use Committee (IACUC) guidelines of the Boston Children's Hospital, Boston, Mass.

### Mouse Studies

For all experiments, 6-8-week-old female Nu/Nu nude mice were purchased from Massachusetts General Hospital. At least 4 days of acclimation was conducted prior to the start of the experiments. For the brain metastasis studies, mice were randomly divided into 3 groups to receive retro-orbital injections of EVs derived from parental and brain-seeking MDA-MB-231 cells (3 μg EVs in 100 μl PBS per injection) or 100 μl of PBS. Injections were conducted every other day, for a total of 10 injections. Following this pretreatment with EVs, intracardiac injections of the brain-seeking MDA-MB-231 cells (2×10⁵ cells in 100 μl HBSS) into the left ventricle were conducted to establish brain metastasis. Four weeks after intracardiac injection, mice were sacrificed and brain tissues were collected and fixed in 4% paraformaldehyde. For histological analysis of brain metastasis, each brain was cut into 5 coronal sections (bread-loafing technique), from which, five 200-μm stepwise sections (10-μm-thick) were prepared, for a total of 25 sections for each brain. Following hematoxylin and eosin (H&E) staining, the presence of macrometastases and micrometastases was evaluated in brain sections in a blind manner by Dr. Roderick Bronson, Rodent Pathology Core, Harvard Medical School.

For distribution studies, 3 μg of TdTom-Br-EVs in 100 μl of PBS were injected retro-orbitally. After 45 min, mice underwent transcardial perfusion with 25 ml of PBS. Brains were embedded in TISSUE-PLUS™ O.C.T. compound (Thermo Fisher Scientific) and frozen in liquid nitrogen. Frozen sections were immunostained with an anti-GFAP antibody (1:100, Abcam, Cat #53554) and DAPI and evaluated for the uptake of Br-EVs by astrocytes, using a Zeiss Fluorescent microscope. To evaluate the integrity of the BBB during this experiment, a combination of 10 KDa Dextran, DQ™ 647 (300 μg), and 70 KDa FITC Dextran (2 mg), with or without 3 μg of Br-EVs in 100 μl of PBS were injected retro-orbitally. Following 45 min, perfusion was performed with 25 ml of PBS. Collected brains were snap-frozen in liquid nitrogen for tissue lysate preparation. Brain tissue lysates were prepared in T-PER™ Tissue Protein Extraction Reagent supplemented with Halt™ protease inhibitor cocktail (Thermo Scientific) using 0.9-2.0 mm stainless steel bead blend (Next Advance Inc.). Fluorescence intensity was measured using a SpectraMax M2 plate reader (Molecular Devices, Inc.) and was normalized to tissue weight. Homogenates form brain tissue of non-treated mice were used to measure the tissue autofluorescence.

For functional studies, mice were randomly divided into 3 groups and received retro-orbital injections of PBS, P-EVs, or Br-EVs (3 μg in 100 μl PBS per injection). Injections were repeated every two days for a total of 10 injections, following which the mice were sacrificed and brain tissue was collected for analysis. For each brain, the right hemisphere was fixed in 10% formalin. Formalin-fixed and paraffin-embedded tissue sections were analyzed using anti-TIMP-2 antibody (1:1000, Servicebio, Cat #GB11523) and anti-GFAP antibody (1:1000, Servicebio, Cat #GB11096). The left hemisphere was snap-frozen in liquid nitrogen. Tissue homogenates were prepared as described above. The expression of MMPs and TIMPs were evaluated using enzyme-linked immunosorbent assays (ELISA) for MMP-2 (R&D Systems Inc. Cat #MMP200), MMP-9 (R&D Systems Inc. Cat #MMPT90), MMP-14 (Lifespan Biosciences Inc., Cat #LS-F7353), TIMP-1 (R&D Systems Inc. Cat #MTM100), and TIMP-2 (Abcam, Cat #ab100746). Except for the MMP-2 ELISA kit that could recognize both human and mouse MMP-2, all kits were mouse-specific. All assays were conducted according to manufacturer's protocol.

### Zebrafish Studies

Tg(kdrl:GFP) zebrafish were used. Embryos were incubated in E3 medium at 28.5° C. and experiments were performed at 6-7 days post-fertilization (dpf). Embryos were anesthetized using tricane (160 μg/ml, Sigma) and were mounted laterally in 0.8% low melting point agarose (ThermoFisher Scientific, Cat #16520050). For transcytosis experiments, intracardiac injection of TdTom-Br-EVs (5 nL of a 400 μg/ml suspension per injection) was performed using the Narishige Injection System. One hour post-injection, live imaging of embryos was conducted using a Nikon Eclipse Ti inverted microscope with a Yokogawa spinning disk scan head and an Andor iXon EM-CCD camera. To evaluate the integrity of the BBB, intracardiac injection of 5 nL of a combination of unlabeled Br-EVs (400 μg/ml), 10 KDa Dextran, ALEXA FLUOR™ 647 (60 μg/ml) and 70 KDa Rhodamin B-Dextran (60 μg/ml, Thermo Fisher Scientific) was performed (n=3-6 zebrafish, 3 independent experiments) and z-stack images of the brain region were taken 1 hour post-injection. To quantify the permeability of the BBB, the mean fluorescence intensity of an intravascular area and the adjacent extravascular area were measured in 5 different locations in the brain of each zebrafish using the ImageJ software. The ratio of intravascular/extravascular fluorescence intensity was calculated as a measure of BBB permeability.

### Western Blot Analyses and ELISA

Cells were lysed with lysis buffer (Cell Signaling Technology, Danvers, Mass.), supplemented with Phenylmethylsulfonyl fluoride protease inhibitor. Following centrifugation of the lysates at 14,000 g for 10 minutes at 4° C., the supernatant was collected for western blot (30 μg total protein/lane). EV samples resuspended in PBS were directly used for western blot (15 μg total protein/lane). Protein concentration was measured using the Bradford method (Biorad laboratories, CA). Immunoblotting was conducted as reported previously⁷⁰. Antibodies against the following proteins were used for immunoblotting: CD63 (1:500, Abcam, Cat #59479), CD9 (1:500, Cell Signaling Technologies, Cat #13174), Alix (1:1000, Cell Signaling Technology, Cat #2171S), and GM130 (1:1000, Cell Signaling Technologies, Cat #12480), Rab 7 (1:1000, Abcam, Cat #137029), Rab 11 (1:250, Cell Signaling Technology, Cat #5589S).

For ELISA, serum-free conditioned media collected from cell cultures was centrifuged at 400 g for 10 minutes at 4° C. to remove the dead cells and debris and the supernatant was used for ELISA. Brain tissue lysates were prepared as described above and were used for human TIMP-2 ELISA (R&D Systems Inc. Cat #DTM200), according to manufacturer's protocol.

### Immunocyto/Histochemistry

For immunocytochemistry, cells were fixed with 10% formalin for 10 minutes and then permeabilized with triton 0.1% triton X-100 for 5 minutes. For immunohistochemical staining, frozen sections was were fixed with ice-cold acetone for 10 minutes. Blocking was performed using 3% bovine serum albumin for 30 minutes. Cells or tissue sections were incubated with the primary antibody for 1 hour at room temperature or overnight at 4° C., respectively. Following washes, cells or tissue sections were incubated with the relevant secondary antibody (1:200) for 1 hour. Sections were washed and mounted with Fluoro-gel mounting medium (Electron Microscopy Sciences). Images were taken using a Zeiss Axiocam fluorescent microscope.

### Statistical Analyses

All quantified data are presented as mean±SD from 3 independent experiments. For animal experiments, the minimum number of animals required to obtain data amenable to statistical analysis was used. Animals were randomly divided into groups. Blinded analyses were only conducted to evaluate the presence of brain metastasis.

All statistical analyses were performed using the GraphPad Prism software. Statistical significance was considered at P values lower than 0.05. P values were shown as * P≤0.05; ** P≤0.01; *** P≤0.001; **** P≤0.0001. No outliers were excluded. The methods of statistical analyses have been indicated in figure legends. All comparisons between two experimental groups were performed by unpaired two-tailed Student's t-test. Comparisons between more than 2 groups were performed by one-way ANOVA with Tukey's correction for multiple comparisons. Groups of data involving more than one variable were analyzed by two-way ANOVA with Sidak's correction for multiple comparisons. All mouse experiments were evaluated using the Mann-Whitney test.

### References for Example 1

1. Cagney, D. N. et al. Incidence and prognosis of patients with brain metastases at diagnosis of systemic malignancy: a population-based study. Neuro Oncol 19, 1511-1521 (2017).
2. Martin, A. M. et al. Brain Metastases in Newly Diagnosed Breast Cancer: A Population-Based Study. JAMA Oncol 3, 1069-1077 (2017).
3. Donnem, T. et al. Vessel co-option in primary human tumors and metastases: an obstacle to effective anti-angiogenic treatment? Cancer Med 2, 427-436 (2013).
4. Carbonell, W. S., Ansorge, O., Sibson, N. & Muschel, R. The vascular basement membrane as “soil” in brain metastasis. PLoS One 4, e5857 (2009).
5. Peinado, H. et al. Pre-metastatic niches: organ-specific homes for metastases. Nat Rev Cancer 17, 302-317 (2017).
6. Costa-Silva, B. et al. Pancreatic cancer exosomes initiate pre-metastatic niche formation in the liver. Nat Cell Biol 17, 816-826 (2015).
7. Peinado, H. et al. Melanoma exosomes educate bone marrow progenitor cells toward a pro-metastatic phenotype through MET. Nat Med 18, 883-891 (2012).
8. Hood, J. L., San, R. S. & Wickline, S. A. Exosomes released by melanoma cells prepare sentinel lymph nodes for tumor metastasis. Cancer Res 71, 3792-3801 (2011).
9. Fong, M. Y. et al. Breast-cancer-secreted miR-122 reprograms glucose metabolism in premetastatic niche to promote metastasis. Nat Cell Biol 17, 183-194 (2015).
10. Morad G, O. H., Dillon S T, Moses M A. in American Association for Cancer Research Annual Meeting 2018, Vol. 78 (AACR; Cancer Res Chicago, Ill.; 2018).
11. Morad G, Y. J., Moses M A in American Association for Cancer Research Annual Meeting 2017, Vol. 77 (AACR; Cancer Res Washington, D.C.; 2017).
12. Tominaga, N. et al. Brain metastatic cancer cells release microRNA-181c-containing extracellular vesicles capable of destructing blood-brain barrier. Nat Commun 6, 6716 (2015).
13. Zhou, W. et al. Cancer-secreted miR-105 destroys vascular endothelial barriers to promote metastasis. Cancer Cell 25, 501-515 (2014).
14. Chow, B. W. & Gu, C. The molecular constituents of the blood-brain barrier. Trends Neurosci 38, 598-608 (2015).
15. Abbott, N.J., Ronnback, L. & Hansson, E. Astrocyte-endothelial interactions at the blood-brain barrier. Nat Rev Neurosci 7, 41-53 (2006).
16. Armulik, A. et al. Pericytes regulate the blood-brain barrier. Nature 468, 557-561 (2010).
17. Banks, W. A. Characteristics of compounds that cross the blood-brain barrier. BMC Neurol 9 Suppl 1, S3 (2009).
18. Yoneda, T., Williams, P. J., Hiraga, T., Niewolna, M. & Nishimura, R. A bone-seeking clone exhibits different biological properties from the MDA-MB-231 parental human breast cancer cells and a brain-seeking clone in vivo and in vitro. J Bone Miner Res 16, 1486-1495 (2001).
19. Thery, C., Amigorena, S., Raposo, G. & Clayton, A. Isolation and characterization of exosomes from cell culture supernatants and biological fluids. Curr Protoc Cell Biol Chapter 3, Unit 3 22 (2006).
20. Thery, C. et al. Minimal information for studies of extracellular vesicles 2018 (MISEV2018): a position statement of the International Society for Extracellular Vesicles and update of the MISEV2014 guidelines. J Extracell Vesicles 7, 1535750 (2018).
21. Weksler, B. B. et al. Blood-brain barrier-specific properties of a human adult brain endothelial cell line. FASEB J 19, 1872-1874 (2005).
22. Rubin, L. L. et al. A cell culture model of the blood-brain barrier. J Cell Biol 115, 1725-1735 (1991).
23. Macia, E. et al. Dynasore, a cell-permeable inhibitor of dynamin. Dev Cell 10, 839-850 (2006).
24. Tae-Eun Park, N. M., Anna Herland, Ryan M Hasselkus, Robert Mannix, Edward A FitzGerald, Rachelle Prantil-Baun, Alexander Watters, Olivier Henry, Maximilian Benz, Henry Sanchez, Heather J McCrea, Liliana Christova Goumnerova, Hannah W Song, Sean P Palecek, Eric Shusta, Donald E Ingber Hypoxia-enhanced Blood-Brain Barrier Chip recapitulates human barrier function, drug penetration, and antibody shuttling properties. BioRxiv (2018).
25. Jeong, J. Y. et al. Functional and developmental analysis of the blood-brain barrier in zebrafish. Brain Res Bull 75, 619-628 (2008).
26. Dutta, D. & Donaldson, J. G. Search for inhibitors of endocytosis: Intended specificity and unintended consequences. Cell Logist 2, 203-208 (2012).
27. Chi, X., Wang, S., Huang, Y., Stamnes, M. & Chen, J. L. Roles of rho GTPases in intracellular transport and cellular transformation. Int J Mol Sci 14, 7089-7108 (2013).
28. Mayle, K. M., Le, A. M. & Kamei, D. T. The intracellular trafficking pathway of transferrin. Biochim Biophys Acta 1820, 264-281 (2012).
29. Li, L. et al. The effect of the size of fluorescent dextran on its endocytic pathway. Cell Biol Int 39, 531-539 (2015).
30. Cullen, P. J. & Steinberg, F. To degrade or not to degrade: mechanisms and significance of endocytic recycling. Nat Rev Mol Cell Biol 19, 679-696 (2018).
31. Nayak, R. C., Keshava, S., Esmon, C. T., Pendurthi, U. R. & Rao, L. V. Rab GTPases regulate endothelial cell protein C receptor-mediated endocytosis and trafficking of factor VIIa. PLoS One 8, e59304 (2013).
32. Weisz, O. A. & Rodriguez-Boulan, E. Apical trafficking in epithelial cells: signals, clusters and motors. J Cell Sci 122, 4253-4266 (2009).
33. Jovic, M., Sharma, M., Rahajeng, J. & Caplan, S. The early endosome: a busy sorting station for proteins at the crossroads. Histol Histopathol 25, 99-112 (2010).
34. Latz, E. et al. TLR9 signals after translocating from the ER to CpG DNA in the lysosome. Nat Immunol 5, 190-198 (2004).
35. Duewell, P. et al. NLRP3 inflammasomes are required for atherogenesis and activated by cholesterol crystals. Nature 464, 1357-1361 (2010).
36. Wang, T., Li, L. & Hong, W. SNARE proteins in membrane trafficking. Traffic 18, 767-775 (2017).
37. Fader, C. M., Sanchez, D. G., Mestre, M. B. & Colombo, M. I. TI-VAMP/VAMP7 and VAMP3/cellubrevin: two v-SNARE proteins involved in specific steps of the autophagy/multivesicular body pathways. Biochim Biophys Acta 1793, 1901-1916 (2009).
38. Torres, J., Funk, H. M., Zegers, M. M. & ter Beest, M. B. The syntaxin 4 N terminus regulates its basolateral targeting by munc18c-dependent and -independent mechanisms. J Biol Chem 286, 10834-10846 (2011).
39. Fields, I. C. et al. v-SNARE cellubrevin is required for basolateral sorting of AP-1B-dependent cargo in polarized epithelial cells. J Cell Biol 177, 477-488 (2007).
40. Fang, J. et al. Matrix metalloproteinase-2 is required for the switch to the angiogenic phenotype in a tumor model. Proc Natl Acad Sci USA 97, 3884-3889 (2000).
41. Harper, J. & Moses, M. A. Molecular regulation of tumor angiogenesis: mechanisms and therapeutic implications. EXS, 223-268 (2006).
42. Overall, C. M. & Kleifeld, O. Tumour microenvironment—opinion: validating matrix metalloproteinases as drug targets and anti-targets for cancer therapy. Nat Rev Cancer 6, 227-239 (2006).
43. Roy R, M. G., Jedinak A, Moses M A Metalloproteinases and their Roles in Human Cancer. Anatomical Records In press (2019).
44. Liu, Y. & Cao, X. Characteristics and Significance of the Pre-metastatic Niche. Cancer Cell 30, 668-681 (2016).
45. Mendes, O., Kim, H. T. & Stoica, G. Expression of MMP2, MMP9 and MMP3 in breast cancer brain metastasis in a rat model. Clin Exp Metastasis 22, 237-246 (2005).
46. Mendes, O., Kim, H. T., Lungu, G. & Stoica, G. MMP2 role in breast cancer brain metastasis development and its regulation by TIMP2 and ERK1/2. Clin Exp Metastasis 24, 341-351 (2007).
47. Liu, H. et al. The role of MMP-1 in breast cancer growth and metastasis to the brain in a xenograft model. BMC Cancer 12, 583 (2012).
48. Wu, K. et al. Roles of the cyclooxygenase 2 matrix metalloproteinase 1 pathway in brain metastasis of breast cancer. J Biol Chem 290, 9842-9854 (2015).
49. Rojiani, M. V., Ghoshal-Gupta, S., Kutiyanawalla, A., Mathur, S. & Rojiani, A. M. TIMP-1 overexpression in lung carcinoma enhances tumor kinetics and angiogenesis in brain metastasis. J Neuropathol Exp Neurol 74, 293-304 (2015).
50. Wang, L. et al. Astrocytes directly influence tumor cell invasion and metastasis in vivo. PLoS One 8, e80933 (2013).
51. Rempe, R. G., Hartz, A. M. S. & Bauer, B. Matrix metalloproteinases in the brain and blood-brain barrier: Versatile breakers and makers. J Cereb Blood Flow Metab 36, 1481-1507 (2016).
52. Fernandez, C. A., Butterfield, C., Jackson, G. & Moses, M. A. Structural and functional uncoupling of the enzymatic and angiogenic inhibitory activities of tissue inhibitor of metalloproteinase-2 (TIMP-2): loop 6 is a novel angiogenesis inhibitor. J Biol Chem 278, 40989-40995 (2003).
53. Guerra, F. & Bucci, C. Multiple Roles of the Small GTPase Rab7. Cells 5 (2016).
54. Sun, Y., Buki, K. G., Ettala, O., Vaaraniemi, J. P. & Vaananen, H. K. Possible role of direct Rac1-Rab7 interaction in ruffled border formation of osteoclasts. J Biol Chem 280, 32356-32361 (2005).
55. Margiotta, A., Progida, C., Bakke, O. & Bucci, C. Rab7a regulates cell migration through Rac1 and vimentin. Biochim Biophys Acta Mol Cell Res 1864, 367-381 (2017).
56. Mascia, A. et al. Rab7 Regulates CDH1 Endocytosis, Circular Dorsal Ruffles Genesis, and Thyroglobulin Internalization in a Thyroid Cell Line. J Cell Physiol 231, 1695-1708 (2016).
57. Malecz, N. et al. Synaptojanin 2, a novel Rac1 effector that regulates clathrin-mediated endocytosis. Curr Biol 10, 1383-1386 (2000).
58. Palmieri, D., Chambers, A. F., Felding-Habermann, B., Huang, S. & Steeg, P. S. The biology of metastasis to a sanctuary site. Clin Cancer Res 13, 1656-1662 (2007).
59. Fidler, I. J., Yano, S., Zhang, R. D., Fujimaki, T. & Bucana, C. D. The seed and soil hypothesis: vascularisation and brain metastases. Lancet Oncol 3, 53-57 (2002).
60. Lyle, L. T. et al. Alterations in Pericyte Subpopulations Are Associated with Elevated Blood-Tumor Barrier Permeability in Experimental Brain Metastasis of Breast Cancer. Clin Cancer Res 22, 5287-5299 (2016).
61. Willms, E. et al. Cells release subpopulations of exosomes with distinct molecular and biological properties. Sci Rep 6, 22519 (2016).
62. Bobrie, A., Colombo, M., Krumeich, S., Raposo, G. & Thery, C. Diverse subpopulations of vesicles secreted by different intracellular mechanisms are present in exosome preparations obtained by differential ultracentrifugation. J Extracell Vesicles 1 (2012).
63. Zhang, H. et al. Identification of distinct nanoparticles and subsets of extracellular vesicles by asymmetric flow field-flow fractionation. Nat Cell Biol 20, 332-343 (2018).
64. Lo, R. W., Li, L., Leung, R., Pluthero, F. G. & Kahr, W. H. A. NBEAL2 (Neurobeachin-Like 2) Is Required for Retention of Cargo Proteins by alpha-Granules During Their Production by Megakaryocytes. Arterioscler Thromb Vasc Biol 38, 2435-2447 (2018).
65. Shelke, G. V., Lasser, C., Gho, Y. S. & Lotvall, J. Importance of exosome depletion protocols to eliminate functional and RNA-containing extracellular vesicles from fetal bovine serum. J Extracell Vesicles 3 (2014).
66. Lai, C. P. et al. Visualization and tracking of tumour extracellular vesicle delivery and RNA translation using multiplexed reporters. Nat Commun 6, 7029 (2015).
67. Lai, C. P. et al. Dynamic biodistribution of extracellular vesicles in vivo using a multimodal imaging reporter. ACS Nano 8, 483-494 (2014).
68. Choudhury, A. et al. Rab proteins mediate Golgi transport of caveola-internalized glycosphingolipids and correct lipid trafficking in Niemann-Pick C cells. J Clin Invest 109, 1541-1550 (2002).
69. Jaskolski, F., Mulle, C. & Manzoni, O. J. An automated method to quantify and visualize colocalized fluorescent signals. J Neurosci Methods 146, 42-49 (2005).
70. Roy, R., Wewer, U. M., Zurakowski, D., Pories, S. E. & Moses, M. A. ADAM 12 cleaves extracellular matrix proteins and correlates with cancer status and stage. J Biol Chem 279, 51323-51330 (2004).

### Example 2 Cdc42-Dependent Transfer of mir301 from Breast Cancer-Derived Extracellular Vesicles Regulates the Matrix Modulating Ability of Astrocytes at the Blood Brain Barrier

It is widely acknowledged that tumor-derived extracellular vesicles (EVs) can promote tumor progression and metastasis. Once released into the circulation, these nanoscale vesicles can transfer their contents including proteins, lipids, DNA, and coding and non-coding RNA to cells in distant organs⁵. The resulting alterations in the behavior of these cells change the microenvironment in pre-metastatic organs in a manner that promotes future metastatic growth⁶⁻⁸. In the brain, the role of tumor-derived EVs in the progression of primary brain tumors has been studied extensively, however the current knowledge of their role in metastatic brain tumors is still limited⁹. EVs have demonstrated great promise as novel diagnostics and therapeutics for a variety of pathologies¹⁰. Understanding the role of breast cancer-derived EVs in brain metastasis can therefore provide opportunities for early detection and management of this disease. It has been demonstrated that EVs derived from brain-seeking breast cancer cell lines (Br-EVs) can promote brain metastasis¹¹⁻¹⁴ It has been shown that these EVs can breach an intact BBB via a transcytosis process in vivo¹¹. Importantly, following their transcytosis, Br-EVs were taken up by astrocytes at the BBB, a process that is of potential mechanistic significance for the observed Br-EV-driven promotion of brain metastasis. As described herein, astrocytes were focused on as one of the major recipients of breast cancer-derived EVs in the brain¹¹, and the mechanisms underlying the uptake of breast cancer-derived EVs by these cells and the associated functional consequences were sought.

It was demonstrated that astrocyte uptake of breast cancer-derived EVs relies on the Cdc42-dependent clathrin-independent carriers/GPI-AP enriched compartments (CLIC/GEEC) endocytic pathway. Using quantitative proteomics analysis, the enrichment of a protein signature with the potential to interact with the CLIC/GEEC cargo was demonstrated. Next, it was demonstrated that the uptake of Br-EVs by astrocytes changes the expression profile of astrocytes to prepare a tumor-supporting microenvironment at the BBB. Data suggesting that at least one mechanism by which this process occurs is through alterations in the expression of extracellular matrix (ECM)-remodeling proteins by astrocytes was presented. Data supportive of the role of miR-301a in EV-driven down-regulation of TIMP-2 was also presented.

### Results

### The Astrocyte Uptake of Breast Cancer-Derived EVs is Mediated Through the CLIC/GEEC Pathway

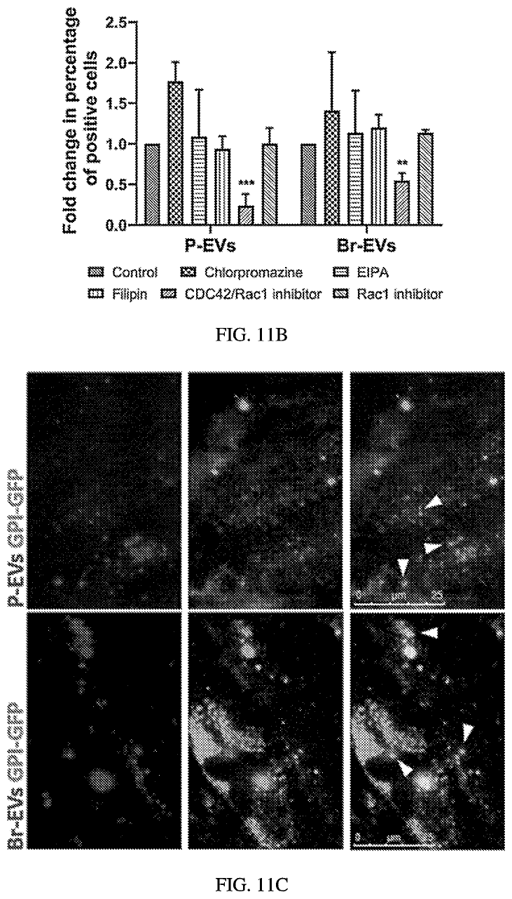

Given the high incidence of brain metastasis in triple negative breast cancer¹, EVs from the human triple negative MDA-MB-231 cell line for which matched primary and brain-seeking variants are available (P-EVs and Br-EVs, respectively) were isolated. Breast cancer-derived EVs were characterized according to the guidelines suggested by the International Society for Extracellular Vesicles¹⁵ (FIG. 11A). It was previously demonstrated that astrocytes are one of the major recipients of Br-EVs in the brain in vivo¹¹, suggesting a prominent role for EV-astrocyte interactions in breast cancer brain metastasis.

To elucidate the mechanisms underlying the uptake of breast cancer-derived EVs by astrocytes, the possibility of the involvement of specific endocytosis mechanism(s) in this uptake was explored. Endocytic pathways such as macropinocytosis, clathrin-dependent and caveolin-dependent endocytosis have been commonly reported to be involved in the uptake of EVs by different cell types¹⁶. Interestingly, using different chemical inhibitors of endocytosis pathways, it was found that none of these common pathways were involved in the uptake of EVs by astrocytes (FIG. 11B). This finding was also in contrast to previous findings demonstrating the involvement of macropinocytosis and clathrin-dependent endocytosis in the uptake of Br-EVs by brain endothelial cells¹¹ and emphasizes the cell-type dependency of EV uptake mechanisms.

Next, the role of clathrin-caveolin-independent pathways in EV uptake was explored, focusing on rac1 and Cdc42, two major players in this process¹⁷. It was found that a Cdc42/Rac1 GTPase Inhibitor, ML141, significantly decreased the uptake of both types of EVs by astrocytes, whereas a specific Rac1 inhibitor, CAS 1177865-17-6, had no effect on their uptake, suggesting that Cdc42, but not Rac1, is involved in the uptake of breast cancer-derived EVs (FIG. 11B).

Cdc42 is known to be involved in the endocytosis of glycosylphosphatidylinositol-anchored proteins (GPI-Aps) via the clathrin-independent carriers/GPI-AP enriched compartments (CLIC/GEEC) pathway¹⁷. To evaluate whether EV uptake by astrocytes occurs through the Cdc42-dependent CLIC/GEEC pathway, astrocytes were transfected with a GFP-fused GPI construct¹⁸. High spatiotemporal resolution microscopy demonstrated the colocalization of TdTomato-labeled EVs with GPI (FIG. 11C), confirming that the breast cancer-derived EVs share the endocytic pathway with GPI-APs.

Taken together, these findings demonstrated that the uptake of breast cancer-derived EVs by astrocytes is mediated through the non-canonical Cdc42-dependent CLIC/GEEC endocytosis pathway.

### Br-EVs are Enriched in Interacting Partners of the CLIC/GEEC Cargo

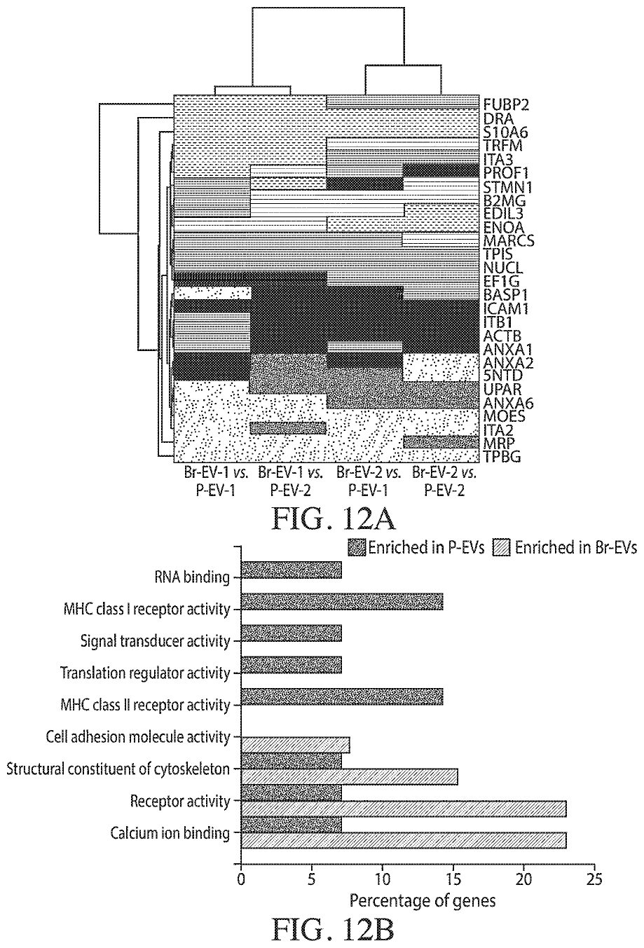

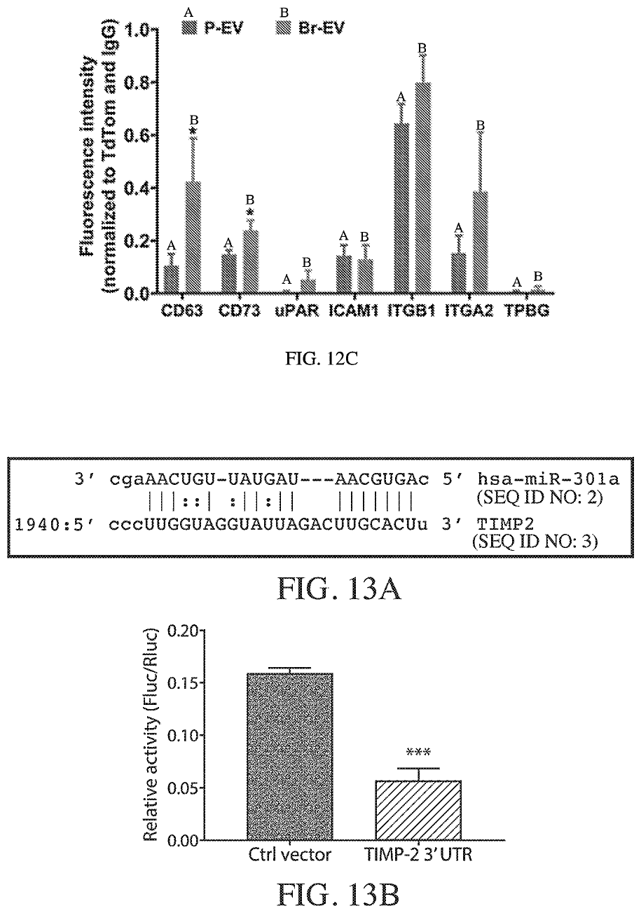

The endocytosis of EVs by different cell types is defined by the composition of surface proteins on EVs and their interaction with receptors and ligands on the cell membrane¹⁹. To identify the composition of proteins on breast cancer-derived EVs, performed first was a quantitative mass spectrometry using the isobaric tag for relative and absolute quantitation (iTRAQ) technique on the two types of breast cancer-derived EVs. Among a total of 126 proteins detected with >95% confidence, 27 proteins were significantly (P≤0.05) differentially expressed (14 upregulated, 13 downregulated) in Br-EVs compared to P-EVs (FIG. 12A). Enrichment analysis using the FunRich software²⁰ demonstrated that the majority of these proteins belonged to receptor activity and cell adhesion categories (FIG. 12B), supporting their involvement in the specific interaction between breast cancer-derived EVs and astrocytes. The surface localization of these proteins was validated and quantified on P- and Br-EVs through staining of the intact EVs (FIG. 12C).

Interestingly, a number of the surface proteins upregulated in Br-EVs have been previously identified as cargoes associated with the CLIC/GEEC pathway. While GPI-APs are the most studied cargo of the CLIC/GEEC pathway, a variety of other proteins, predominantly adhesion factors, have also been identified as the cargo of this endocytosis route. These include integrin (31, galectin 3, CD44, and CD98²¹⁻²³. Moreover, it has been shown that ICAM1 binding to integrins can induce nucleation and colocalization of integrin clusters and GPI-APs²³. It was demonstrated that Br-EVs were enriched in Ecto-5′-nucleotidase (5NTD, also known as CD73) and urokinase plasminogen activator receptor (uPAR), well-known GPI-interacting proteins^{24,25}, as well as integrin β1 and integrin α2. Both types of EVs had similar expression of ICAM1 on their surface (FIG. 12C). CD63 was included as a control. Together, these findings identify a combination of surface proteins upregulated in Br-EVs that have the potential to interact with GPI-AP clusters. These results are consistent with previous findings demonstrating a preferential uptake of Br-EVs compared to P-EVs by astrocytes in vitro¹¹. Moreover, the enrichment of Br-EVs in surface proteins that can facilitate their internalization by astrocytes provides a potential explanation as to why Br-EVs but not P-EVs have the ability to promote brain metastasis, as has been previously reported^{11,14}.

### Br-EVs Decrease the Astrocyte Expression of TIMP-2

Next, the functional consequences of EV uptake by astrocytes in vivo were studied. Upon transcytosis through the brain endothelium, breast cancer-derived EVs may change the behavior of astrocytes to prepare a microenvironment supportive of tumor cell growth. It is widely acknowledged that matrix metalloproteinases (MMPs) and their endogenous inhibitors, the tissue inhibitors of MMPs (TIMPs) can contribute to tumor progression and metastasis²⁶⁻²⁹. Through modulating the ECM, these enzymes and their inhibitors can trigger different signaling pathways and promote the tumor-supporting microenvironment^{5,30}. Several studies have demonstrated a prominent role for MMPs and TIMPs in preparation of a niche for tumor cell growth in the brain³¹⁻³⁵ While these studies predominantly focus on tumor cell-derived MMPs and TIMPs, astrocyte conditioned media was shown to modulate the tumor cell expression of MMPs. Moreover, astrocyte-derived MMP-2 and MMP-9, have also been shown to promote tumor cell invasion in breast cancer brain metastasis³⁶. Accordingly, it was postulated that Br-EVs can alter the expression of MMPs and TIMPs produced by astrocytes to facilitate brain metastasis. To address this, retro-orbital injections of P-EVs and Br-EVs (3 μg in 100 μl PBS per injection) were performed in mice every two days for a total of 10 injections, following which the mice were sacrificed to analyze the brain tissue (FIG. 4A). Retro-orbital injection is considered to be a superior route of administration for continuous injections by IACUC and is commonly used for the injection of EVs into the circulation 6,37 Using mouse-specific enzyme-linked immunosorbent assays (ELISA), the expression of a number of MMPs and TIMPs that are known to be involved in ECM remodeling in brain tissue³⁸ were analyzed, including MMP-2, MMP-9, MMP-14, TIMP-1, and TIMP-2 in mouse brain tissue homogenates (FIG. 4B). Interestingly, it was found that TIMP-2, the endogenous inhibitor of MMP activity³⁹, was exclusively and significantly decreased by brain metastasis-promoting Br-EVs but not P-EVs (FIG. 4B).

It was investigated whether astrocytes could be the source of the Br-EV-driven decrease in brain TIMP-2. Human BBB cells, endothelial cells, pericytes, and astrocytes, were treated with P-EVs and Br-EVs in vitro and evaluated TIMP-2 expression using a human-specific TIMP-2 ELISA. EV treatment did not affect the expression of TIMP-2 in brain endothelial cells (FIG. 4C) but decreased the expression of TIMP-2 in astrocytes (FIG. 4C). Moreover, Br-EVs were able to increase the migration of astrocytes, which is consistent with the observed decrease in TIMP-2 expression and a subsequent increase in ECM modulation. This finding supports that Br-EVs can change the behavior of astrocytes. To rule out the possibility that the decreased astrocyte TIMP-2 might be an indirect effect of Br-EVs acting through brain endothelial cells, conditioned media was prepared by treating brain endothelial cells with EVs or PBS. Astrocytes were incubated with the endothelial cell conditioned media for 48 hours, followed by the media exchange and subsequent analysis of the astrocyte conditioned media. No difference in TIMP-2 levels was found in conditioned media from astrocytes that were incubated with PBS-, P-EV-, or Br-EV-treated endothelial cell conditioned media. In addition, consecutive brain tissue sections were stained for TIMP-2 and an astrocyte marker, GFAP, and found that areas that were rich in astrocytes also had a high expression of TIMP-2, further supporting astrocytes as the major source of TIMP-2 (FIG. 4D).

Then, it was evaluated whether the observed decrease in astrocyte TIMP-2 levels following Br-EV treatment were accompanied by alterations in the permeability of the BBB. No increase in permeability of brain endothelium to 10 KDa- and 70 KDa-dextran was observed following P- or Br-EV treatment (FIG. 4E). This observation suggested that the BBB remained intact during this experiment, supporting the conclusion that the EV-induced decrease in TIMP-2 was a direct effect of transcytosed Br-EVs on astrocytes. Overall, these findings indicate that the transcytosis of Br-EVs and their subsequent uptake by astrocytes can have functional consequences, such as suppressed TIMP-2 expression, that can lead to the preparation of a microenvironment at the BBB suitable for the growth of metastases.

Interestingly, both P-EVs and Br-EVs were able to induce TIMP-2 down-regulation in vitro whereas in vivo, this effect was exclusive to Br-EVs. These findings indicate that both EVs have the inherent ability to down-regulate TIMP-2 in astrocytes, with Br-EVs being able to reach the astrocytes more efficiently in vivo. The results with respect to the enrichment of Br-EVs in GPI-interacting proteins (FIGS. 12A-12C), along with the previous findings on the ability of Br-EVs to facilitate their transcytosis across the BBB¹¹, is consistent with the findings described herein.

miR-301a-3p Transferred by Breast Cancer-Derived EVs Downregulate TIMP-2 in Astrocytes

To determine the EV factors driving the decrease in TIMP-2, the role of EV miRNAs in this process was examined. Previous reports have identified a number of miRNAs with the ability to target the 3′UTR of TIMP-2 mRNA in tumor cells, including miR-106a⁴⁰, miR-761⁴¹ and miR-301a⁴². Interestingly, in a whole miRNome analysis conducted by the group, treatment of brain endothelial cells by breast cancer-derived EVs increased the miR-301a-3p levels, suggesting the ability of breast cancer-derived EVs to transfer this miRNA into recipient cells. This observation prompted us to investigate the potential role of miR-301a-3p in the observed EV-driven down-regulation of TIMP-2 in astrocytes.

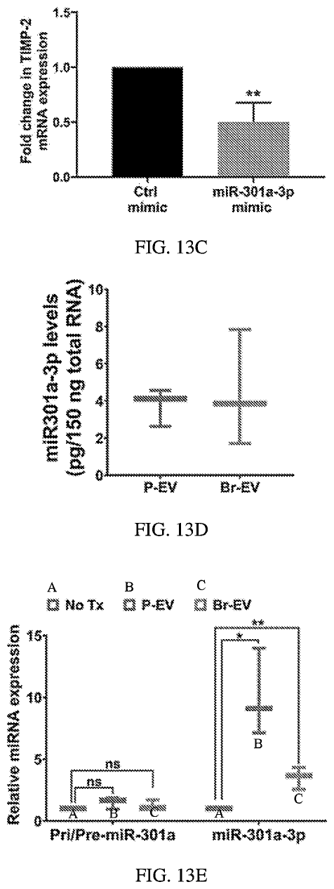

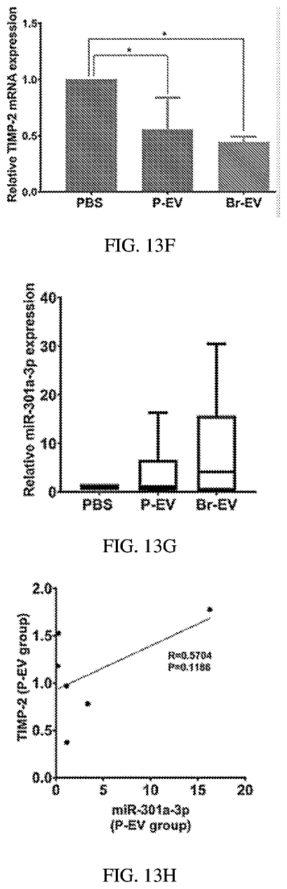

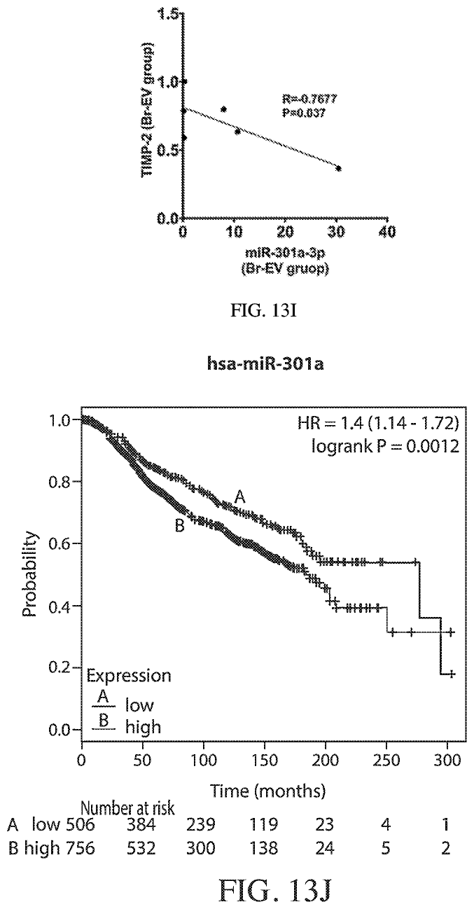

Computational target prediction tools (miroRNA.org) demonstrated perfect complementarity between the miR-301a-3p seeding sequence and the TIMP-2 3′ UTR (FIG. 13A). The ability of miRNAs to induce functional effects can differ based on how different cell types process EVs and their miRNA content⁴³. To examine the ability of miR-301a-3p to physically interact with the 3′ UTR of TIMP-2 in astrocytes, the cells were transfected with a dual luciferase reporter vector of TIMP-2 3′ UTR or a control vector. miR-301a-3p mimic significantly decreased the luminescence activity in the TIMP-2 3′UTR-transfected cells, validating TIMP-2 as a target for miR-301a-3p (FIG. 13B). Treatment of astrocytes with miR-301a-3p mimic also led to a decrease in endogenous TIMP-2 mRNA levels (FIG. 13C), demonstrating the functionality of this miRNA in astrocytes.

To examine whether breast cancer-derived EVs carry this miRNA, the miR-301a-3p levels in P- and Br-EVs were measured and it was found that both types of EVs carried similar amounts of this miRNA (FIG. 13D). To determine the ability of breast cancer-derived EVs to transfer this miRNA to astrocytes astrocytes were treated with EVs and measured the alterations in the levels of miR-301a-3p in astrocytes. Treatment of astrocytes with P- and Br-EVs led to an increase in miR-301a-3p, demonstrating the transfer from EVs to astrocytes (FIG. 13E). Furthermore, the levels of primary and precursor miR-301a were not changed following EV treatment, confirming that the observed increase in mature miRNA was not due to upregulation of endogenous miRNA in astrocytes and was a result of direct transfer from EVs. As expected, this increase in miR-301a-3p was associated with a down-regulation of TIMP-2 mRNA (FIG. 13F). Together, these findings demonstrated that breast cancer-derived EVs transfer miR-301a-3p to astrocytes, which can then directly target and downregulate TIMP-2 in these cells.

To evaluate the ability of breast cancer-derived EVs to transfer this miRNA to the brain in vivo, the level of miR-301a-3p in brain tissues collected from the in vivo experiment described above was analyzed. It is important to note that the conserved and identical sequence of miR-301a-3p in mouse and human limited the ability to detect and analyze the direct transfer of miR-301a-3p by human breast cancer-derived EVs. Nevertheless, an increasing trend in the levels of miR-301a-3p was observed in mice that were treated with Br-EVs (FIG. 13G). Importantly, the level of miR-301a-3p was significantly and negatively correlated with the level of TIMP-2 in Br-EV-treated mice, whereas this correlation was not observed in P-EV-treated mice (FIGS. 13H and 131). These studies demonstrated a correlation between the level of miR-301a-3p and the observed down-regulation of TIMP-2 in vivo. Given that Br-EV-driven down-regulation of astrocyte TIMP-2 can occur prior to brain metastasis formation, miR-301a-3p has the potential to serve as a diagnostic marker for early stages of brain metastasis. Interestingly, analysis of 1262 patients in the METABRIC (Molecular Taxonomy of Breast Cancer International Consortium⁴⁴) dataset, demonstrated that higher levels of miR-301a-3p were significantly associated with decreased survival (kmplot.com, FIG. 13J).

### Discussion

As described herein, the functional consequences of transcellular transport of breast cancer-derived EVs across the BBB were explained, with a focus on the interaction of these EVs with astrocytes. A series of mechanisms were identified through which EVs are internalized by, and modulate, the behavior of astrocytes to promote a microenvironment supportive of metastatic growth.

It was demonstrated that astrocytes internalize breast cancer-derived EVs through the specific Cdc42-dependent CLIC/GEEC pathway. This study is the first to report the uptake of EVs through this endocytosis pathway^{16,45} Interestingly, it has been shown that adeno-associated viruses can hijack the CLIC/GEEC pathway to gain entry into cells⁴⁶. These findings are consistent with previous report of EVs using the virus entry machinery to enter cells⁴⁷. Cells can internalize EVs through a variety of pathways including non-specific pathways (fusion, macropinocytosis) and receptor-mediated pathways. The uptake of EVs through receptor-mediated pathways is attributed to the interaction of EV surface proteins with ligands/receptors on the cell membrane¹⁶. However, the significant heterogeneity of EV populations suggests that multiple EV surface proteins are likely involved in the uptake of EVs by a particular cell type. Through a combination of proteomics analyses and localization studies, a group of proteins were identified, enriched on the surface of brain metastasis-promoting breast cancer-derived EVs. These proteins were recognized as interacting counterparts of several CLIC/GEEC pathway cargoes and therefore can play significant roles in the uptake of Br-EVs by astrocytes. Future studies incorporating these proteins into synthetic nanoparticles can evaluate the necessity and significance of each of these proteins for internalization by astrocytes. Collectively, the identified protein signature can define a subpopulation of breast cancer-derived EVs that have the ability to interact with astrocytes and, in doing so, provide novel opportunities to address the longstanding challenge of dismantling the heterogeneity of EVs for identification of functional subpopulations.

Through in vitro and in vivo functional studies, it was further demonstrated that Br-EVs can downregulate TIMP-2 in astrocytes. While the role of matrix metalloproteinases and their endogenous inhibitors in progression of metastasis has been studied extensively²⁹, this study is the first to demonstrate that tumor-derived EVs can initiate this process in the brain and provides insight into the early mechanisms involved in priming a niche prior to brain metastasis.

miR-301a-3p was identified as the causal factor that can be transferred by breast cancer-derived EVs to astrocytes and down-regulate TIMP-2. Interestingly, while the previous¹¹ and current studies on the in vitro uptake of EVs by astrocytes suggest a preferential uptake of Br-EVs by these cells, it was found that, at a functional level, both P-EVs and Br-EVs carried similar amounts of this miRNA and were able to induce similar effects on TIMP-2 in vitro. These discrepancies are most likely due to the different duration of the functional experiments conducted, during which cells had continuous and direct access to both types of EVs for a longer time allowing them to reach the functional threshold. The limited resolution of the currently available technologies does not allow for reliable assessment of the preferential uptake of Br-EVs compared to P-EVs by astrocytes in vivo. However, it was found that despite the inherent ability of both types of EVs to downregulate TIMP-2 in astrocytes, this effect was only observed following treatment with Br-EVs but not P-EVs in vivo. The specificity in the function of Br-EVs in vivo can potentially be explained by a higher efficiency of Br-EVs to reach the astrocytes in vivo. Importantly, it was shown that Br-EVs, but not P-EVs, have the ability to modulate brain endothelial cells to facilitate their transcellular transport to reach astrocytes on the abluminal side¹¹. More efficient internalization of Br-EVs by astrocytes could be another potential explanation.

Taken together, these studies uncover novel mechanisms by which breast cancer-derived EVs prime the microenvironment in the brain following their transcytosis across the BBB. These mechanisms provide novel insights into the early events that occur prior to brain metastasis development from triple negative breast cancer. It is important to note that the literature regarding triple negative breast cancer brain metastasis is currently limited to the use of available matched primary and brain-metastatic cell lines. Development of transgenic models of spontaneous brain metastasis is critical for a better understanding of the mechanisms underlying brain metastasis.

The identified protein and miRNA signatures in this study have the potential to guide the development of diagnostics and therapeutics that would enable early interventions in triple negative breast cancer brain metastasis. Future longitudinal preclinical studies and prospective clinical studies are required to validate the clinical implications of these findings.

### Materials and Methods

### Cell Lines and Cell Culture

Human breast cancer cell line MDA-MB-231 was purchased from American Type Culture Collection (ATCC® HTB-26™, VA, USA). The brain-seeking variant of the breast cancer cell line MDA-MB-231 was a gift from Dr. T. Yoneda, Indiana University⁴⁸. Primary human brain microvascular endothelial cells, astrocytes, and human brain vascular pericytes were purchased from Cell Systems Co. (Cat #ACBRI 376, Kirkland, Wash.), Thermo Fisher Scientific Inc. (Cat #N7805100), and ScienCell Research Laboratories (Cat #1200, Carlsbad, Calif.), respectively.

Breast cancer cells were cultured in Dulbecco's Modified Eagle's medium (DMEM, Cat #11885084, Thermo Fisher Scientific Inc.) supplemented with 10% fetal bovine serum (FBS, Cat #S11150, Atlanta Biologicals™, Atlanta, Ga., USA) and 1% Penicillin-Streptomycin (10,000 U/mL) (Cat #15140148, Thermo Fisher Scientific Inc.). For extracellular vesicle (EV) isolation, breast cancer cells were cultured in Advanced DMEM supplemented with 10% EV-depleted FBS. EV-depleted FBS-containing medium was prepared as described previously^{11,49}. Human brain endothelial cells were cultured with endothelial cell growth medium (EGM™-2MV, Cat #CC-3202, Lonza Inc., ME, USA). Human astrocytes and brain pericytes were cultured according to the manufacturer's instructions. All cells were maintained in a 37° C. humidified incubator with 5% CO2. All cultures were routinely monitored for mycoplasma contamination using the MycoAlert™ PLUS Mycoplasma Detection Kit (Cat #LT07-710, Lonza Inc.).

### EV Isolation and Characterization

EVs were isolated from 24-48-h conditioned media from breast cancer cell cultures with >95% cell viability, using a sequential centrifugation technique¹¹. Briefly, conditioned media was centrifuged at 400×g for 10 min, 2000×g for 15 min, and 15000×g for 30 min at 4° C. (Sorvall® RC-5B centrifuge, Thermo Fisher Scientific Inc.) followed by ultracentrifugation at 100000×g for 90 min at 4° C. (Optima XE-90 Ultracentrifuge, Beckman Coulter Life Sciences). EV pellets were washed at 100000×g for another 90 min and were resuspended in PBS.

EV preparations were characterized according to the guidelines of the International Society for Extracellular Vesicles¹⁵ and as described previously by us¹¹. EV size and concentration was measured by nanoparticle tracking analysis (NanoSight NS300, Malvern Instruments, UK). EV markers were evaluated by western blot and the shape of the EVs was evaluated by electron microscopy¹¹.

To isolate TdTomato-labeled EVs, breast cancer cells were transduced with a lentiviral vector to express palmitoylated TdTomato (PalmtdTomato)⁵⁰. The DNA construct was a gift of Dr. X. Breakefield, Massachusetts General Hospital. The fluorescence label of the isolated EVs were evaluated by fluorescent microscopy and plate reader (SpectraMax M2 plate reader, Molecular Devices, Inc.)

### In Vitro EV Uptake Studies

To evaluate the uptake of EVs by astrocytes for endocytosis inhibition studies, astrocytes were treated with chlorpromazine hydrochloride (Millipore Sigma, Cat #C8138, 20 μM), 5-(N-Ethyl-N-isopropyl) amiloride (EIPA) (50 μM, Tocris, Cat #3378), and filipin III (10 μM, Millipore Sigma, Cat #F4767), CDC42/Rac1 inhibitor, ML141 (100 μM, Millipore Sigma, Cat #217708), and rac1 inhibitor, CAS 1177865-17-6 (10 μM, Millipore Sigma, Cat #553502) for 30 min. Subsequently, TdTom-Br-EVs (10¹⁰ particle/well in a 12-well plate) were incubated with astrocytes for 3 hours, following which, cells were washed and EV uptake was measured by flow cytometry using a BD FACSCalibur flow cytometer (BD Biosciences, San Jose, Calif.).

To evaluate the colocalization of EVs with GPI-APs in astrocytes, cells were initially transfected with GFP-GPI plasmid using Lipofectamine 3000 reagent (Thermo Fisher Scientific). GFP-GPI WT plasmid was a gift of Dr. A. K. Hadjantonakis (Addgene plasmid #32601; http://n2t.net/addgene:32601; RRID:Addgene_32601)¹⁸. Transfected cells were cultured on glass-bottom microslides (Ibidi, Cat #80827) and were incubation with TdTom-Br-EVs (8×109 particles/well) for 30 min. Subsequently, cells were washed 4 times with PBS and fixed with 4% formaldehyde for 10 min. Epifluorescence microscopy was performed on a Leica microscope coupled to high-resolution objectives and a Hamamatsu Orca CCD (Japan).

### In Vitro EV Functional Studies

To evaluate the direct effect of EVs on the expression profile of BBB cells, primary human brain ECs, astrocytes, and pericytes cultured in 12-well plates were treated with P- or Br-EVs for 48 h (25 μg EVs per treatment). Conditioned media were collected for downstream analyses. The amount of TIMP-2 was measured in conditioned media using a human TIMP-2 ELISA (R&D Systems Inc. Cat #DTM200) according to the manufacturer's protocol.

For astrocyte migration studies following continuous EV treatment, astrocytes were trypsinized and were plated in Transwell filters (8 μm pores, Costar Transwell Assay; Corning Inc., Corning N.Y.) in astrocyte serum-free media (2×10⁴ cells in 100 μl media per filter). Filters were placed in 24-well plates containing 600 μl of complete astrocyte media. After 16 h, cells were fixed and stained with DAPI. Cells attached to the top of the filter were removed. Membranes were separated from the filters and were mounted on glass slides. The number of cells on the bottom of the filters were counted using a Zeiss Axiocam fluorescent microscope at 200× magnification (4 fields/filter, n=2 filters per treatment).

To assess any indirect effects of EVs on astrocytes, brain ECs were initially treated with P- or Br-EVs for 3 consecutive days (5 μg EVs per treatment), as previously described¹¹. After treatment, EC conditioned media was collected. Astrocytes cultured in 6-well plates were incubated with EC conditioned media for 48 hours, following which the cells were serum starved overnight and the astrocyte conditioned media was collected for analysis. TIMP-2 levels were measured using a human TIMP-2 ELISA as described above.

### Validation of EV Surface Proteins

Tdtomato P-EVs and Br-EVs (10¹⁰ particles in 100 μl PBS) were incubated with 5 μg/ml of fluorescent-conjugated antibodies: FITC anti-human CD73 antibody (BioLegend, Cat #344015), FITC anti-human uPAR antibody (Sino Biological, Cat #10925-MM09-F), Alexa Flour® 488 anti-human ICAM1/CD54 antibody (BioLegend, Cat #322713), Alexa Flour® 488 anti-human CD29 antibody (BioLegend, Cat #303015), FITC anti-human CD49b antibody (BioLegend, Cat #359305), and Alexa Flour® 488 anti-human CD63 antibody (BioLegend, Cat #353037), Alexa Flour® 488 mouse IgG1, κ isotype control (BioLegend, Cat #400132), and FITC mouse IgG1, κ isotype control (BioLegend, Cat #400107). Following a 2-h incubation at room temperature, EVs were washed through ultracentrifugation to remove any free antibodies. Pellets were resuspended in PBS and fluorescence intensity was measured using a SpectraMax M2 plate reader. The fluorescence intensity of FITC or Alexa Flour® 488 was normalized to that of TdTomato for both antibodies and isotype controls. The normalized measurements of antibodies were then subtracted from those of isotype controls.

### Proteomics Analysis

Quantitative proteomics analysis was performed using the isobaric tags for relative and absolute quantitation (iTRAQ) technique as was described previously⁵¹. For protein identification, the peak list was searched against the Swiss-Prot database including all human proteins. Both detection and differential expression analyses were carried out using the ProteinPilot software (AB SCIEX). An unbiased ProtScore of >1.3, which corresponds to 95% confidence in detection (P<0.05) was used for analysis. Significantly differentially expressed proteins between two samples were identified based on the ratio of the protein expression levels in the two samples (P<0.05). Hierarchical clustering of samples and features was done using the Unweighted Pair Group Method with Arithmetic-mean (UPGMA) method with Pearson's correlation as the distance measure⁵². The expression data matrix was row-normalized prior to the application of average linkage clustering. Functional enrichment analysis of the proteins that were enriched in Br-EVs was performed using the FunRich software²⁰.

### In Vivo Experiments

All animal experiments were conducted in accordance with the Institutional Animal Care and Use Committee (IACUC) guidelines of the Boston Children's Hospital, Boston, Mass.

Six to eight-week-old female Nu/Nu nude mice were purchased from Massachusetts General Hospital. Following 4-7 days of acclimation, mice were randomly divided into 3 groups and received retro-orbital injections of PBS, P-EVs or Br-EVs (3 μg in 100 μl PBS per injection). Injections were repeated every two days for a total of 10 injections, following which the mice were sacrificed and brain tissue was collected for analysis. For each brain, the left hemisphere was snap-frozen in liquid nitrogen. Tissue homogenates were prepared as described above. The expression of MMPs and TIMPs were evaluated using ELISAs for MMP-2 (R&D Systems Inc. Cat #MMP200), MMP-9 (R&D Systems Inc. Cat #MMPT90), MMP-14 (Lifespan Biosciences Inc., Cat #LS-F7353), TIMP-1 (R&D Systems Inc. Cat #MTM100), and TIMP-2 (Abcam, Cat #ab100746). All kits were mouse-specific, except for the MMP-2 ELISA kit that could recognize both human and mouse MMP-2. All assays were conducted according to manufacturers' protocols. The right hemisphere was fixed in 10% formalin. Formalin-fixed and paraffin-embedded tissue sections were analyzed using anti-TIMP-2 antibody (1:1000, Servicebio, Cat #GB11523) and anti-GFAP antibody (1:1000, Servicebio, Cat #GB11096). Immunohistochemistry was conducted as previously described¹¹.

To evaluate the integrity of the BBB during this experiment, the experiment was conducted as described above. Following the 3-week EV treatment, at the time of sacrifice, mice received a retro-orbital injection of a combination of 10 KDa Dextran, Alexa Fluor™ 647 (300 μg), and 70 KDa FITC Dextran (2 mg), in 100 μl of PBS. Following 45 min, perfusion was performed with 25 ml of PBS. Collected brains were snap-frozen in liquid nitrogen for tissue lysate preparation. Brain tissue lysates were prepared in T-PER™ Tissue Protein Extraction Reagent supplemented with Halt™ protease inhibitor cocktail (Thermo Scientific) using 0.9-2.0 mm stainless steel bead blend (Next Advance Inc.). Fluorescence intensity was measured using a SpectraMax M2 plate reader and was normalized to tissue weight. Homogenates form brain tissue of non-treated mice were used to measure the tissue autofluorescence.

miRNA Target Validation

For target validation, astrocytes were transfected with dual luciferase reporters (TIMP-2 and control clones, GeneCopoeia™, Cat #HmiT018093-MT06, and CmiT000001-MT06, respectively). Following 48 hours, cells were transfected with miRNA-301a-3p mimics (50 nM miRIDIAN, Dharmacon Inc.) using the Dharmafect 4 transfection reagent. Luciferase assays were conducted 48 h after mimic treatment, using the Luc-Pair™ Duo-Luciferase HS Assay Kit (GeneCopoeia™), according to manufacturer's instructions. For functional evaluation of miR-301a-3p, astrocytes were treated with 50 nM miRNA-301a-3p mimics for 48 h after which RNA was isolated for analysis.

### RNA Isolation and Analysis

RNA isolation from EV samples, astrocytes, and brain tissue was conducted using the miRNeasy kit (Qiagen), according to the manufacturer's protocol. Brain tissues were homogenized in Qiazol reagent using the stainless steel bead blend, as described previously. For analysis of TIMP-2 mRNA expression, mature miRNA expression and miRNA precursor analyses, the SuperScript™ VILO™ cDNA Synthesis Kit and the SYBR™ Green PCR Master Mix (ThermoFisher Scientific) were used, miRCURY LNA RT Kit and SYBR Green PCR kit (Qiagen), and the miScript II RT kit and SYBR Green PCR kit (Qiagen), respectively. The following primers were used for these studies: PrimePCR™ SYBR® Green Assay: TIMP2, Human (Bio-Rad, assay ID qHsaCID0022953); miRCury LNA miRNA PCR assays (U6 snRNA-hsa, hsa-miR-301a-3p, hsa-miR-301b-3p) and Hs_miR-301a_1_PR miScript precursor assay, and Hs_miR-301a_1 and Hs_RNU6-2_11 miScript primer assays (Qiagen).

### Statistical Analyses

Statistical analyses were performed using the GraphPad Prism software. All quantified data are presented as mean±SD from 3 independent experiments. Statistical significance was considered at P values lower than 0.05. P values were shown as * P≤0.05; ** P≤0.01; *** P≤0.001; **** P≤0.0001. The methods of statistical analyses have been indicated in figure legends. All comparisons between two experimental groups were performed by unpaired two-tailed Student's t-test. Comparisons between more than 2 groups were performed by one-way ANOVA with Tukey's correction for multiple comparisons. Groups of data involving more than one variable were analyzed by two-way ANOVA with Sidak's correction for multiple comparisons. For in vivo experiments, the minimum number of animals required to conduct statistical analysis were included in the study and were randomly assigned into experimental groups. All in vivo experiments were evaluated using the Mann-Whitney test. The correlation between miR-301a-3p and TIMP-2 levels was evaluated via Pearson's correlation test.

### References for Example 2

1. Cagney, D. N., et al. Incidence and prognosis of patients with brain metastases at diagnosis of systemic malignancy: a population-based study. Neuro Oncol 19, 1511-1521 (2017).
2. Martin, A. M., et al. Brain Metastases in Newly Diagnosed Breast Cancer: A Population-Based Study. JAMA Oncol 3, 1069-1077 (2017).
3. Donnem, T., et al. Vessel co-option in primary human tumors and metastases: an obstacle to effective anti-angiogenic treatment? Cancer Med 2, 427-436 (2013).
4. Carbonell, W. S., Ansorge, O., Sibson, N. & Muschel, R. The vascular basement membrane as “soil” in brain metastasis. PLoS One 4, e5857 (2009).
5. Peinado, H., et al. Pre-metastatic niches: organ-specific homes for metastases. Nat Rev Cancer 17, 302-317 (2017).
6. Costa-Silva, B., et al. Pancreatic cancer exosomes initiate pre-metastatic niche formation in the liver. Nat Cell Biol 17, 816-826 (2015).
7. Peinado, H., et al. Melanoma exosomes educate bone marrow progenitor cells toward a pro-metastatic phenotype through MET. Nat Med 18, 883-891 (2012).
8. Hood, J. L., San, R. S. & Wickline, S. A. Exosomes released by melanoma cells prepare sentinel lymph nodes for tumor metastasis. Cancer Res 71, 3792-3801 (2011).
9. Morad, G. & Moses, M. A. Brainwashed by extracellular vesicles: the role of extracellular vesicles in primary and metastatic brain tumour microenvironment. J Extracell Vesicles 8, 1627164 (2019).
10. Fais, S., et al. Evidence-Based Clinical Use of Nanoscale Extracellular Vesicles in Nanomedicine. ACS Nano 10, 3886-3899 (2016).
11. Morad, G., et al. Tumor-Derived Extracellular Vesicles Breach the Intact Blood-Brain Barrier via Transcytosis. ACS Nano 13, 13853-13865 (2019).
12. Maji, S., et al. Exosomal Annexin II Promotes Angiogenesis and Breast Cancer Metastasis. Mol Cancer Res 15, 93-105 (2017).
13. Tominaga, N., et al. Brain metastatic cancer cells release microRNA-181c-containing extracellular vesicles capable of destructing blood-brain barrier. Nat Commun 6, 6716 (2015).
14. Rodrigues, G., et al. Tumour exosomal CEMIP protein promotes cancer cell colonization in brain metastasis. Nat Cell Biol 21, 1403-1412 (2019).
15. Thery, C., et al. Minimal information for studies of extracellular vesicles 2018 (MISEV2018): a position statement of the International Society for Extracellular Vesicles and update of the MISEV2014 guidelines. J Extracell Vesicles 7, 1535750 (2018).
16. Mulcahy, L. A., Pink, R. C. & Carter, D. R. Routes and mechanisms of extracellular vesicle uptake. J Extracell Vesicles 3(2014).
17. Chi, X., Wang, S., Huang, Y., Stamnes, M. & Chen, J. L. Roles of rho GTPases in intracellular transport and cellular transformation. Int J Mol Sci 14, 7089-7108 (2013).
18. Rhee, J. M., et al. In vivo imaging and differential localization of lipid-modified GFP-variant fusions in embryonic stem cells and mice. Genesis 44, 202-218 (2006).
19. Abels, E. R. & Breakefield, X. O. Introduction to Extracellular Vesicles: Biogenesis, RNA Cargo Selection, Content, Release, and Uptake. Cell Mol Neurobiol 36, 301-312 (2016).
20. Pathan, M., et al. A novel community driven software for functional enrichment analysis of extracellular vesicles data. J Extracell Vesicles 6, 1321455 (2017).
21. Howes, M. T., et al. Clathrin-independent carriers form a high capacity endocytic sorting system at the leading edge of migrating cells. J Cell Biol 190, 675-691 (2010).
22. Maldonado-Baez, L., Williamson, C. & Donaldson, J. G. Clathrin-independent endocytosis: a cargo-centric view. Exp Cell Res 319, 2759-2769 (2013).
23. Lakshminarayan, R., et al. Galectin-3 drives glycosphingolipid-dependent biogenesis of clathrin-independent carriers. Nat Cell Biol 16, 595-606 (2014).
24. Strater, N. Ecto-5′-nucleotidase: Structure function relationships. Purinergic Signal 2, 343-350 (2006).
25. Lakhan, S. E., Sabharanjak, S. & De, A. Endocytosis of glycosylphosphatidylinositol-anchored proteins. J Biomed Sci 16, 93 (2009).
26. Fang, J., et al. Matrix metalloproteinase-2 is required for the switch to the angiogenic phenotype in a tumor model. Proc Natl Acad Sci USA 97, 3884-3889 (2000).
27. Harper, J. & Moses, M. A. Molecular regulation of tumor angiogenesis: mechanisms and therapeutic implications. EXS, 223-268 (2006).
28. Overall, C. M. & Kleifeld, O. Tumour microenvironment—opinion: validating matrix metalloproteinases as drug targets and anti-targets for cancer therapy. Nat Rev Cancer 6, 227-239 (2006).
29. Roy, R., Morad, G., Jedinak, A., Moses, M. A. Metalloproteinases and their Roles in Human Cancer. Anatomical Records In press (2019).
30. Liu, Y. & Cao, X. Characteristics and Significance of the Pre-metastatic Niche. Cancer Cell 30, 668-681 (2016).
31. Mendes, O., Kim, H. T. & Stoica, G. Expression of MMP2, MMP9 and MMP3 in breast cancer brain metastasis in a rat model. Clin Exp Metastasis 22, 237-246 (2005).
32. Mendes, O., Kim, H. T., Lungu, G. & Stoica, G. MMP2 role in breast cancer brain metastasis development and its regulation by TIMP2 and ERK1/2. Clin Exp Metastasis 24, 341-351 (2007).
33. Liu, H., et al. The role of MMP-1 in breast cancer growth and metastasis to the brain in a xenograft model. BMC Cancer 12, 583 (2012).
34. Wu, K., et al. Roles of the cyclooxygenase 2 matrix metalloproteinase 1 pathway in brain metastasis of breast cancer. J Biol Chem 290, 9842-9854 (2015).
35. Rojiani, M. V., Ghoshal-Gupta, S., Kutiyanawalla, A., Mathur, S. & Rojiani, A. M. TIMP-1 overexpression in lung carcinoma enhances tumor kinetics and angiogenesis in brain metastasis. J Neuropathol Exp Neurol 74, 293-304 (2015).
36. Wang, L., et al. Astrocytes directly influence tumor cell invasion and metastasis in vivo. PLoS One 8, e80933 (2013).
37. Lai, C. P., et al. Dynamic biodistribution of extracellular vesicles in vivo using a multimodal imaging reporter. ACS Nano 8, 483-494 (2014).
38. Rempe, R. G., Hartz, A. M. S. & Bauer, B. Matrix metalloproteinases in the brain and blood-brain barrier: Versatile breakers and makers. J Cereb Blood Flow Metab 36, 1481-1507 (2016).
39. Fernandez, C. A., Butterfield, C., Jackson, G. & Moses, M. A. Structural and functional uncoupling of the enzymatic and angiogenic inhibitory activities of tissue inhibitor of metalloproteinase-2 (TIMP-2): loop 6 is a novel angiogenesis inhibitor. J Biol Chem 278, 40989-40995 (2003).
40. Zhu, M., et al. MicroRNA-106a targets TIMP2 to regulate invasion and metastasis of gastric cancer. FEBS Lett 588, 600-607 (2014).
41. Yan, A., Yang, C., Chen, Z., Li, C. & Cai, L. MiR-761 Promotes Progression and Metastasis of Non-Small Cell Lung Cancer by Targeting ING4 and TIMP2. Cell Physiol Biochem 37, 55-66 (2015).
42. Liang, B., Yin, J. J. & Zhan, X. R. MiR-301a promotes cell proliferation by directly targeting TIMP2 in multiple myeloma. Int J Clin Exp Pathol 8, 9168-9174 (2015).
43. O'Brien, J., Hayder, H., Zayed, Y. & Peng, C. Overview of MicroRNA Biogenesis, Mechanisms of Actions, and Circulation. Front Endocrinol (Lausanne) 9, 402 (2018).
44. Curtis, C., et al. The genomic and transcriptomic architecture of 2,000 breast tumours reveals novel subgroups. Nature 486, 346-352 (2012).
45. McKelvey, K. J., Powell, K. L., Ashton, A. W., Morris, J. M. & McCracken, S. A. Exosomes: Mechanisms of Uptake. J Circ Biomark 4, 7 (2015).
46. Nonnenmacher, M. & Weber, T. Adeno-associated virus 2 infection requires endocytosis through the CLIC/GEEC pathway. Cell Host Microbe 10, 563-576 (2011).
47. Yao, Z., et al. Exosomes Exploit the Virus Entry Machinery and Pathway To Transmit Alpha Interferon-Induced Antiviral Activity. J Virol 92(2018).
48. Yoneda, T., Williams, P. J., Hiraga, T., Niewolna, M. & Nishimura, R. A bone-seeking clone exhibits different biological properties from the MDA-MB-231 parental human breast cancer cells and a brain-seeking clone in vivo and in vitro. J Bone Miner Res 16, 1486-1495 (2001).
49. Shelke, G. V., Lasser, C., Gho, Y. S. & Lotvall, J. Importance of exosome depletion protocols to eliminate functional and RNA-containing extracellular vesicles from fetal bovine serum. J Extracell Vesicles 3(2014).
50. Lai, C. P., et al. Visualization and tracking of tumour extracellular vesicle delivery and RNA translation using multiplexed reporters. Nat Commun 6, 7029 (2015).
51. Jedinak, A., et al. Novel non-invasive biomarkers that distinguish between benign prostate hyperplasia and prostate cancer. BMC Cancer 15, 259 (2015).
52. Sneath, P. Numerical taxonomy; the principles and practice of numerical classification, (W. H. Freeman, San Francisco, Calif. USA, 1973).

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of treating a brain disease, the method comprising administering to a subject in need thereof an effective amount of an extracellular vesicle (EV) comprising a therapeutic agent for the brain disease and a Rab7 GTPase (Rab7) inhibitor.

2. The method of claim 1, wherein the EV is isolated from a cell.

3. The method of claim 2, wherein the cell is a stem cell, a bone marrow derived cell, an immune cell, a red blood cell, an epithelial cell, or an endothelial cell.

4. The method of claim 2, wherein the EV is an engineered EV.

5. The method of claim 1, wherein the EV is an exosome, microvesicle, microparticle, ectosome, oncosome, or apoptotic body.

6. The method of any one of claim 1-5, wherein the EV encapsulates both the therapeutic agent for the brain disease and the Rab7 inhibitor.

7. The method of any one of claims 1-6, wherein the Rab7 inhibitor inhibits Rab7 expression.

8. The method of claim 7, wherein the Rab7 inhibitor comprises an antisense oligonucleotide that targets Rab7 mRNA.

9. The method of claim 8, wherein the anti-sense oligonucleotide is a RNAi molecule.

10. The method of claim 9, wherein the RNAi molecule is a siRNA or miRNA.

11. The method of any one of claims 1-6, wherein the Rab7 inhibitor inhibits Rab7 activity.

12. The method of claim 11, wherein the Rab7 inhibitor is a small molecule inhibitor.

13. The method of any one of claims 1-12, wherein the brain disease is selected from the group consisting of: brain cancer, neurologic disorder, psychological disorder, cerebrovascular vascular disorder, brain trauma, and brain infection.

14. The method of claim 13, wherein the brain disease is brain cancer.

15. The method of claim 14, wherein the brain cancer is primary brain cancer.

16. The method of claim 14, wherein the brain cancer is metastatic brain cancer.

17. The method of any one of claims 14-16, wherein the therapeutic agent is an anti-cancer agent.

18. The method of claim 17, wherein the anti-cancer agent is a chemotherapeutic agent or an immunotherapeutic agent.

19. The method of claim 17, wherein the anti-cancer agent is an RNAi molecule.

20. The method of claim 17, wherein the anticancer agent is a gene-editing agent.

21. The method of any one of claims 17-20, wherein the anticancer agent is an Cdc42 inhibitor.

22. The method of claim 21, wherein the Cdc42 inhibitor is a GTPase inhibitor.

23. The method of any one of claims 17-19, wherein the anticancer agent is a miR301 inhibitor.

24. The method of claim 13, wherein the brain disease is a neurologic disorder.

25. The method of claim 24, wherein the neurologic disorder is a neurodegenerative disease, a neurobehavioral disease, or a developmental disorder.

26. The method of claim 25, wherein the neurodegenerative disease is selected from Alzheimer's disease, Parkinson's disease, Huntington's disease, dementia, amyotrophic lateral sclerosis (ALS), prion disease, and motor neuron disease.

27. The method of any one of claims 24-26, wherein the therapeutic agent is selected from: dopaminergic agent, cholinesterase inhibitor, anti-psychotic drug, anti-inflammatory, and brain stimulant.

28. The method of claim 13, wherein the brain disease is a psychological disorder.

29. The method of claim 28, wherein the psychological disorder is post-traumatic stress disorder (PTSD), depressive disorder, major depressive disorder, post-partum depression, bipolar disorder, acute stress disorder, generalized anxiety disorder, obsessive-compulsive disorder, panic disorder, schizophrenia, or trichotillomania.

30. The method of claim 29, wherein the therapeutic agent is a psychiatric drug.

31. The method of claim 30, wherein the psychiatric drug is selected from anti-depressant, anti-psychotic, mood stabilizer, brain stimulant, and anti-anxiety drug.

32. The method of claim 13, wherein the brain disease is brain trauma.

33. The method of claim 32, wherein the therapeutic agent is selected from: anti-inflammatory agents, corticosteroids, coagulant drug, and anti-coagulant drug.

34. The method of claim 13, wherein the brain disease is brain infection.

35. The method of claim 34, wherein the therapeutic agent is an anti-infective agent.

36. The method of claim 35, wherein the anti-infective agent is selected from: antibiotic, anti-viral agent, anti-fungal agent, anti-parasite agent, and anti-prion antibody.

37. The method of any one of claims 1-36, wherein the EV is administered via injection or infusion.

38. The method of any one of claims 1-37, wherein the EV is administered intravenously, subcutaneously, intraperitoneally, or intracerebrally.

39. The method of any one of claims 1-38, wherein the Rab7 inhibitor increases the transfer of the EV across the blood brain barrier.

40. The method of any one of claims 1-39, wherein the Rab7 inhibitor enhances the uptake of the therapeutic agent by the brain.

41. The method of any one of claims 1-40, wherein the subject is human.

42. A method of delivering an agent to the brain of a subject, the method comprising administering to a subject in need thereof an extracellular vesicle (EV) comprising the agent and a Rab7 GTPase (Rab7) inhibitor.

43. The method of claim 42, wherein the agent is a therapeutic agent or a diagnostic agent.

44. A method of diagnosing a brain disease, the method comprising administering to a subject in need thereof an extracellular vesicle (EV) comprising a diagnostic agent and a Rab7 GTPase (Rab7) inhibitor.

45. A composition comprising an extracellular vesicle (EV) comprising an agent and a Rab7 GTPase (Rab7) inhibitor for delivering the agent to the brain of a subject.

46. The composition of claim 45, wherein the EV is isolated from a cell.

47. The composition of claim 46, wherein the cell is a stem cell, a bone marrow derived cell, an immune cell, a red blood cell, an epithelial cell, or an endothelial cell.

48. The composition of claim 45, wherein the EV is an engineered EV.

49. The composition of claim 45, wherein the EV is an exosome, microvesicle, microparticle, ectosome, oncosome, or apoptotic body.

50. The composition of any one of claim 45-49, wherein the EV encapsulates both the therapeutic agent for the brain disease and the Rab7 inhibitor.

51. The composition of any one of claims 45-50, wherein the Rab7 inhibitor inhibits Rab7 expression.

52. The composition of claim 51, wherein the Rab7 inhibitor comprises an antisense oligonucleotide that targets Rab7 mRNA.

53. The composition of claim 52, wherein the anti-sense oligonucleotide is a RNAi molecule.

54. The composition of claim 53, wherein the RNAi molecule is a siRNA or miRNA.

55. The composition of any one of claims 45-50, wherein the Rab7 inhibitor inhibits Rab7 activity.

56. The composition of claim 55, wherein the Rab7 inhibitor is a small molecule inhibitor.

57. The composition of any one of claims 45-56, wherein the brain disease is selected from the group consisting of: brain cancer, neurologic disorder, psychological disorder, cerebrovascular vascular disorder, brain trauma, and brain infection.

58. The composition of claim 57, wherein the brain disease is brain cancer.

59. The composition of claim 58, wherein the brain cancer is primary brain cancer.

60. The composition of claim 58, wherein the brain cancer is metastatic brain cancer.

61. The composition of any one of claims 58-60, wherein the therapeutic agent is an anti-cancer agent.

62. The composition of claim 61, wherein the anti-cancer agent is a chemotherapeutic agent or an immunotherapeutic agent.

63. The composition of any one of claim 61, wherein the anti-cancer agent is an RNAi molecule.

64. The composition of claim 61, wherein the anticancer agent is a gene-editing agent.

65. The composition of any one of claims 61-64, wherein the anticancer agent is an Cdc42 inhibitor.

66. The composition of claim 65, wherein the Cdc42 inhibitor is a GTPase inhibitor.

67. The composition of any one of claims 61-64, wherein the anticancer agent is an miR301 inhibitor.

68. The composition of claim 57, wherein the brain disease is a neurologic disorder.

69. The composition of claim 68, wherein the neurologic disorder is a neurodegenerative disease, a neurobehavioral disease, or a developmental disorder.

70. The composition of claim 69, wherein the neurodegenerative disease is selected from Alzheimer's disease, Parkinson's disease, Huntington's disease, dementia, amyotrophic lateral sclerosis (ALS), prion disease, and motor neuron disease.

71. The composition of any one of claims 68-70, wherein the therapeutic agent is selected from: dopaminergic agent, cholinesterase inhibitor, anti-psychotic drug, anti-inflammatory, and brain stimulant.

72. The composition of claim 57, wherein the brain disease is a psychological disorder.

73. The composition of claim 72, wherein the psychological disorder is post-traumatic stress disorder (PTSD), depressive disorder, major depressive disorders, post-partum depression, bipolar disorder, acute stress disorder, generalized anxiety disorder, obsessive-compulsive disorder, panic disorder, schizophrenia, or trichotillomania.

74. The composition of claim 73, wherein the therapeutic agent is a psychiatric drug.

75. The composition of claim 74, wherein the psychiatric drug is selected from anti-depressant, anti-psychotic, mood stabilizer, brain stimulant, and anti-anxiety drug.

76. The composition of claim 57, wherein the brain disease is brain trauma.

77. The composition of claim 76, wherein the therapeutic agent is selected from: anti-inflammatory agent, corticosteroid, coagulant drug, and anti-coagulant.

78. The composition of claim 77, wherein the brain disease is brain infection.

79. The composition of claim 78, wherein the therapeutic agent is an anti-infective agent.

80. The composition of claim 79, wherein the anti-infective agent is selected from: antibiotic, anti-viral agent, anti-fungal agent, anti-parasite agent, and anti-prion antibody.

81. The composition of any one of claims 45-80, wherein the EV is administered via injection or infusion.

82. The composition of any one of claims 45-81, wherein the EV is administered intravenously, subcutaneously, intraperitoneally, or intracerebrally.

83. The composition of any one of claims 45-82, wherein the Rab7 inhibitor increases the transfer of the EV across the blood brain barrier.

84. The composition of any one of claims 45-83, wherein the Rab7 inhibitor enhances the uptake of the therapeutic agent by the brain.

85. The composition of any one of 45-84, further comprising a pharmaceutically acceptable carrier.

86. The composition of any one of claims 45-85, wherein the subject is human.

87. Use of the composition of any one of claims 45-86 for treating or diagnosing a brain disease.

88. A method of predicting and/or detecting brain metastasis in a subject having breast cancer, the method comprising isolating an extracellular vesicle (EV) from the subject and detecting in the EV miR-301a-3p, wherein the presence of miR-301a-3p indicates the subject is more likely to develop and/or to have brain metastasis, compared to a subject having breast cancer and an EV where the presence of miR-301a-3p is not detected.

89. A method of predicting and/or detecting brain metastasis in a subject having breast cancer, the method comprising isolating an extracellular vesicle (EV) from the subject and detecting in the EV one or more biomarkers selected from the group consisting of: TPBG, MRP, ITA2, MOES, ANXAS, UPAR, 5NTD, ANXA2, ANXA1, ACTB, ITB1, ICAM1, BASP1, EF1G, STMN1, and PROF1, wherein the presence of one or more of the biomarkers in the EV indicates the subject is more likely to develop and/or to have brain metastasis, compared to a subject having breast cancer and an EV where the presence of the biomarkers is not detected or a lower level is detected.

90. A method of predicting and/or detecting brain metastasis in a subject having breast cancer, the method comprising isolating an extracellular vesicle (EV) from the subject and detecting in the EV one or more biomarkers selected from the group consisting of: TPBG, MRP, ITA2, MOES, ANXAS, UPAR, 5NTD, ANXA2, ANXA1, ACTB, ITB1, ICAM1, BASP1, EF1G, STMN1, PROF1, and miR-301a-3p, wherein the presence of one or more of the biomarkers in the EV indicates the subject is more likely to develop and/or to have brain metastasis, compared to a subject having breast cancer and an EV where the presence of the biomarkers is not detected or a lower level is detected.
